(19) United States
(12) Patent Application Publication
(10) Pub. No.: US 2016/0067336 A1
(43) Pub. Date: Mar. 10, 2016

(54) METHODS FOR TREATING A DISEASE OR DISORDER USING ORAL FORMULATIONS OF CYTIDINE ANALOGS IN COMBINATION WITH AN ANTI-PD1 OR ANTI-PDL1 MONOCLONAL ANTIBODY

(71) Applicant: Celgene Corporation, Summit, NJ (US)

(72) Inventors: Abderrahim FANDI, Pully (CH); David M. REISER, Villanova, PA (US); Debora BARTON, Springfield, NJ (US); Damir BEGIC, Jersey City, NJ (US)

(21) Appl. No.: 14/847,570

(22) Filed: Sep. 8, 2015

Related U.S. Application Data
(60) Provisional application No. 62/047,463, filed on Sep. 8, 2014.

(51) Int. Cl. A61K 39/395; A61K 45/06; A61K 31/706

(52) U.S. Cl. CPC A61K 39/3955; A61K 31/706; A61K 45/06; A61K 2039/505

(57) ABSTRACT

The present disclosure provides methods of treating diseases or disorders with oral cytidine analogs (e.g., 5-azacytidine) in combination with anti-PD1/anti-PDL1 antibodies (e.g., pembrolizumab or durvalumab). The diseases or disorders include, but are not limited to, relapsed or refractory myelodysplastic syndromes, acute myeloid leukemia, ovarian cancer, or non-small cell lung cancer.

This application claims priority to U.S. Provisional application No. 62/047,463, filed Sep. 8, 2014, the entirety of which is incorporated herein by reference.

### I. FIELD

Provided herein are methods for treating a disease or disorder using oral formulations of cytidine analogs, or pharmaceutically acceptable salts, solvates or hydrates thereof, in combination with an anti-PD1 or anti-PDL1 monoclonal antibody. Also provided herein are oral formulations of cytidine analogs, or pharmaceutically acceptable salts, solvates or hydrates thereof, in combination with an anti-PD1 or anti-PDL1 monoclonal antibody that can be used in said methods.

### II. BACKGROUND

Cancer is a major worldwide public health problem; in the United States alone, approximately 570,000 cancer-related deaths were expected in 2005. See, e.g., Jemal et al., CA Cancer J. Clin. 55(1):10-30 (2005). Many types of cancer have been described in the medical literature. Examples include cancer of the blood, bone, lung (e.g., non-small-cell lung cancer and small-cell lung cancer), colon, breast, prostate, ovary, brain, and intestine. The incidence of cancer continues to climb as the general population ages and as new forms of cancer develop. A continuing need exists for effective therapies to treat subjects with cancer.

Acute myeloid leukemia (AML), also known as acute myelogenous leukemia or acute nonlymphocytic leukemia (ANLL), is a cancer of the myeloid line of blood cells, characterized by the rapid growth of abnormal white blood cells that accumulate in the bone marrow and interfere with the production of normal blood cells. AML is the most common acute leukemia affecting adults, and its incidence increases with age.

Myelodysplastic syndromes (MDS) refers to a diverse group of hematopoietic stem cell disorders. MDS affects approximately 40,000-50,000 people in the U.S. and 75,000-85,000 people in Europe. MDS may be characterized by a cellular marrow with impaired morphology and maturation (dysmyelopoiesis), peripheral blood cytopenias, and a variable risk of progression to acute leukemia, resulting from ineffective blood cell production. See, e.g., The Merck Manual 953 (17th ed. 1999); List et al., J. Clin. Oncol. 8:1424 (1990).

MDS are grouped together because of the presence of dysplastic changes in one or more of the hematopoietic lineages including dysplastic changes in the myeloid, erythroid, and megakaryocytic series. These changes result in cytopenias in one or more of the three lineages. Patients afflicted with MDS may develop complications related to anemia, neutropenia (infections), and/or thrombocytopenia (bleeding). From about 10% to about 70% of patients with MDS may develop acute leukemia. In the early stages of MDS, the main cause of cytopenias is increased programmed cell death (apoptosis). As the disease progresses, a proliferation of leukemic cells may overwhelm the healthy marrow. The disease course differs, with some cases behaving as an indolent disease and others behaving aggressively with a clinical course rapidly leading to AML transformation. The majority of people with higher risk MDS eventually experience bone marrow failure. Up to 50% of MDS patients succumb to complications, such as infection or bleeding, before progressing to AML.

Primary and secondary MDS are defined by taking into account patients' prior history: previous treatments with chemotherapy, radiotherapy or professional exposure to toxic substances are factors delineating secondary MDS (sMDS) from primary MDS. Cytogenetically, one difference between the two groups is the complexity of abnormal karyotypes; single chromosome aberrations are typical for primary MDS, while multiple changes are more frequently seen in secondary disorders. Some drugs may have specific targets such as hydroxurea for 17p and topoisomerases inhibitors for 11q23 and 21q22. The genetic changes in the malignant cells of MDS result mainly in the loss of genetic material, including probable tumor suppressor genes.

An international group of hematologists, the French-American-British (FAB) Cooperative Group, classified MDS into five subgroups, differentiating them from acute myeloid leukemia. See, e.g., The Merck Manual 954 (17th ed. 1999); Bennett J. M., et al., Ann. Intern. Med., 103(4): 620-5 (1985); and Besa E. C., Med. Clin. North Am. 76(3): 599-617 (1992). An underlying trilineage dysplastic change in the bone marrow cells of the patients is found in all subtypes. Information is available regarding the pathobiology of MDS, certain MDS classification systems, and particular methods of treating and managing MDS. See, e.g., U.S. Pat. No. 7,189,740 (issued Mar. 13, 2007), which is incorporated by reference herein in its entirety. There exists a great need for treating relapsed or refractory MDS.

Nucleoside analogs have been used clinically for the treatment of viral infections and cancer. Most nucleoside analogs are classified as anti-metabolites. After they enter the cell, nucleoside analogs are successively phosphorylated to nucleoside 5′-mono-phosphates, di-phosphates, and tri-phosphates.

5-Azacytidine (National Service Center designation NSC-102816; CAS Registry Number 320-67-2), also known as azacitidine, AZA, or 4-amino-1-β-D-ribofuranosyl-1,3,5-triazin-2(1H)-one, is currently marketed as the drug product VIDAZA®. 5-Azacytidine is a nucleoside analog, more specifically a cytidine analog. 5-Azacytidine is an antagonist of its related natural nucleoside, cytidine. 5-Azacytidine and 5-aza-2′-deoxycytidine (also known as decitabine, an analog of deoxycytidine) are also antagonists of deoxycytidine. A structural difference between these cytidine analogs and their related natural nucleoside is the presence of a nitrogen at position 5 of the cytosine ring in place of a carbon. 5-Azacytidine may be defined as having the molecular formula C₈H₁₂N₄O₅, a molecular weight of 244.21 grams per mole, and the following structure:

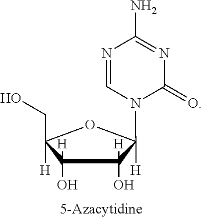

Other members of the class of cytidine analogs include, for example: 1-β-D-arabinofuranosylcytosine (Cytarabine or ara-C); 5-aza-2′-deoxycytidine (Decitabine or 5-aza-CdR); pseudoisocytidine (psi ICR); 5-fluoro-2′-deoxycytidine (FCdR); 2′-deoxy-2′,2′-difluorocytidine (Gemcitabine); 5-aza-2′-deoxy-2′,2′-difluorocytidine; 5-aza-2′-deoxy-2′-fluorocytidine; 1-β-D-ribofuranosyl-2(1H)-pyrimidinone (Zebularine); 2′,3′-dideoxy-5-fluoro-3′-thiacytidine (Emtriva); 2′-cyclocytidine (Ancitabine); 1-β-D-arabinofuranosyl-5-azacytosine (Fazarabine or ara-AC); 6-azacytidine (6-aza-CR); 5,6-dihydro-5-azacytidine (dH-aza-CR); N⁴-pentyloxycarbonyl-5′-deoxy-5-fluorocytidine (Capecitabine); N⁴-octadecyl-cytarabine; and elaidic acid cytarabine.

After its incorporation into replicating DNA, 5-azacytidine or 5-aza-2′-deoxycytidine forms a covalent complex with DNA methyltransferases. DNA methyltransferases are responsible for de novo DNA methylation and for reproducing established methylation patterns in daughter DNA strands of replicating DNA. Inhibition of DNA methyltransferases by 5-azacytidine or 5-aza-2′-deoxycytidine leads to DNA hypomethylation, thereby restoring normal functions to morphologically dysplastic, immature hematopoietic cells and cancer cells by re-expression of genes involved in normal cell cycle regulation, differentiation and death. The cytotoxic effects of these cytidine analogs cause the death of rapidly dividing cells, including cancer cells, that are no longer responsive to normal cell growth control mechanisms. 5-azacytidine, unlike 5-aza-2′-deoxycytidine, also incorporates into RNA. The cytotoxic effects of azacitidine may result from multiple mechanisms, including inhibition of DNA, RNA and protein synthesis, incorporation into RNA and DNA, and activation of DNA damage pathways. [0013]5-Azacytidine and 5-aza-2′-deoxycytidine have been tested in clinical trials and showed significant anti-tumor activity, such as, for example, in the treatment of MDS, AML, chronic myelogenous leukemia (CML), acute lymphocytic leukemia (ALL), and non Hodgkin's lymphoma (NHL). See, e.g., Aparicio et al., Curr. Opin. Invest. Drugs 3(4): 627-33 (2002). 5-Azacytidine has undergone NCI-sponsored trials for the treatment of MDS and has been approved for treating all FAB subtypes of MDS. See, e.g., Kornblith et al., J. Clin. Oncol. 20(10): 2441-2452 (2002); Silverman et al., J. Clin. Oncol. 20(10): 2429-2440 (2002). 5-Azacytidine may alter the natural course of MDS by diminishing the transformation to AML through its cytotoxic activity and its inhibition of DNA methyltransferase. In a Phase III study, 5-azacytidine administered subcutaneously significantly prolonged survival and time to AML transformation or death in subjects with higher-risk MDS. See, e.g., P. Fenaux et al., Lancet Oncol., 2009, 10(3):223-32; Silverman et al., Blood 106(11): Abstract 2526 (2005).

Epigenetic silencing of genes encoding tumor specific antigens, antigen processing and presentation machinery and pro-inflammatory cytokines and chemokines can be reversed in vitro by treatment of tumor cells with sub-cytotoxic concentrations of hypomethylating drugs such as 5-aza-cytidine or 5-aza-2-deoxycytidine (DAC) (Azacitidine Investigator's Brochure). Gene expression changes in response to AZA treatment across a panel of breast, colorectal and epithelial ovarian cancer (EOC) cell lines have been described. AZA IMmune (AIMs) gene signature comprising 317 pro-immunogenic genes was defined. Of the 3 tumor types represented in the panel, EOC lines had the most prevalent and pronounced AIM response to AZA treatment which activated immune response pathways and antigen presentation genes (Li, 2014).

The importance of intact functions of immune surveillance in controlling outgrowth of neoplastic transformations has been widely known. High expression of PD-L1 (programmed death-ligand 1) on tumor cells has been found to correlate with poor prognosis and survival in various cancer types including ovarian carcinoma (Hamanishi, 2007). Preclinical data suggests PD-1 (programmed death-1 or programmed cell death-1) pathway as a viable target in ovarian cancer (Maine, 2014; Duraiswamy 2013). Clinical data on PD-1/PD-L1 inhibition in EOC are limited but in a Phase 1 study of the anti-PD-L1 antibody BMS-936559, one of seventeen patients with EOC had an objective response (Brahmer, 2012).

Inhibition of the immune checkpoint with antibodies directed at the PD-1 receptor on T cells or its ligand (PD-L1) on tumor cells has demonstrated promising anti-tumor activity in a number of tumors, including melanoma and non-small cell lung cancer (Johnson, 2014; Robert 2014).

Pembrolizumab, also known as MK-3475 and Keytruda, is a humanized monoclonal IgG4 antibody directed against human cell surface receptor PD-1 with potential immunopotentiating activity. Upon administration, pembrolizumab binds to PD-1, an inhibitory signaling receptor expressed on the surface of activated T cells, and blocks the binding to and activation of PD-1 by its ligands, which results in the activation of T-cell-mediated immune responses against tumor cells. The ligands for PD-1 include PD-L1, which is expressed on antigen presenting cells (APCs) and overexpressed on certain cancer cells, and PD-L2, which is primarily expressed on APCs. Activated PD-1 negatively regulates T-cell activation through the suppression of the PI3K/Akt pathway.

There still exists a significant unmet need for the treatment of cancers (solid or blood-borne) and hematologic disorders, in particular those that relapse after or are refractory to prior therapeutic treatments. For example, EOC that has relapsed following a platinum based regimen, and MDS or AML that is not responding to treatment with injectable hypomethylating agents, are a significant unmet medical need.

### III. SUMMARY OF INVENTION

Provided herein is a combination of 5-azacytidine, or a pharmaceutically acceptable salt, solvate or hydrate thereof with one or more additional therapeutic agent(s), including an anti-PD1 or anti-PDL1 monoclonal antibody, that can be used in all the methods provided herein.

Provided herein is a compound for use in a method for treating a subject having a disease or disorder, wherein the compound is 5-azacytidine, or a pharmaceutically acceptable salt, solvate or hydrate thereof and wherein the method comprises cyclically administering to the subject a therapeutically effective amount of 5-azacytidine, or a pharmaceutically acceptable salt, solvate or hydrate thereof, and a therapeutically effective amount of an anti-PD1 or anti-PDL1 monoclonal antibody, wherein the 5-azacytidine, or a pharmaceutically acceptable salt, solvate or hydrate thereof is administered orally.

Provided herein is a combination of 5-azacytidine, or a pharmaceutically acceptable salt, solvate or hydrate thereof with one or more additional therapeutic agent(s), including an anti-PD1 or anti-PDL1 monoclonal antibody, that can be used in all the methods for treating, preventing or managing a disease or disorder in a subject. Provided herein are methods for treating, preventing or managing a disease or disorder in a subject, using a cytidine analog (e.g. 5-azacytidine), or a salt, solvate, or hydrate thereof, in combination with one or more additional therapeutic agent(s), including a PD-1 inhibitor. Also provided are methods for using a cytidine analog, or a salt, solvate, or hydrate thereof, in combination with one or more additional therapeutic agent(s) including a PD-1 inhibitor to treat, prevent, or manage diseases and disorders, including disorders related to abnormal cell proliferation, hematologic disorders, and immune disorders, among others. In certain embodiments, the disease or disorder is cancer. In one embodiment, the cancer is relapsed or refractory. In one embodiment, the cancer is a solid tumor. In particular embodiments, the cancer is a relapsed or refractory solid tumor. In a specific embodiment, the cancer is a cancer of the ovary or lung among others. In another embodiment, the cancer is a cancer of the blood or the lymph. In a specific embodiment, the cancer is AML. In yet another embodiment, the hematologic disorder is MDS.

In certain embodiments, the cytidine analog is formulated in an oral dosage form provided herein (e.g., a tablet or a capsule). In one embodiment, the cytidine analog is administered orally to a subject in need thereof. In one embodiment, the cytidine analog is administered to a subject in need thereof for a sustained period of time. In one embodiment, the cytidine analog is administered to a subject in need thereof cyclically (e.g., dosing for one or more days, followed by a resting period). In one embodiment, the cytidine analog is administered to a subject in need thereof over multiple dosing cycles.

In certain embodiments, the PD-1 inhibitor is an anti-PD-1 antibody. In one embodiment, the antibody is a monoclonal antibody. In one embodiment, the antibody is a humanized antibody. In a particular embodiment, the anti-PD-1 monoclonal antibody is pembrolizumab.

In certain embodiments, the cytidine analog is administered orally or parenterally. In a preferred embodiment, the cytidine analog is administered orally. In particular embodiments, 5-azacytidine is administered orally. In one embodiment, the additional therapeutic agent is administered orally or parenterally. In one embodiment, the cytidine analog is administered via the same route as the one or more additional therapeutic agent(s). In one embodiment, the cytidine analog is administered via a different route as the one or more additional therapeutic agent(s) (e.g., one administered orally and the other administered parenterally).

In certain embodiments, the cytidine analog and/or the one or more additional therapeutic agent(s) (including, but not limited to an anti-PD1 or anti-PDL1 monoclonal antibody) are administered in a particular dosing cycle.

In certain embodiments, a method for treating a subject having a disease or disorder, which comprises cyclically administering to the subject a therapeutically effective amount of 5-azacytidine, or a pharmaceutically acceptable salt, solvate or hydrate thereof, and a therapeutically effective amount of an anti-PD1 monoclonal antibody, wherein the 5-azacytidine, or a pharmaceutically acceptable salt, solvate or hydrate thereof is administered orally.

In certain embodiments, a method for treating a subject having a disease or disorder, which comprises cyclically administering to the subject a therapeutically effective amount of 5-azacytidine, or a pharmaceutically acceptable salt, solvate or hydrate thereof, and a therapeutically effective amount of an anti-PDL1 monoclonal antibody, wherein the 5-azacytidine, or a pharmaceutically acceptable salt, solvate or hydrate thereof is administered orally.

In certain embodiments, the disease or disorder is a solid tumor or a hematologic disorder. In specific embodiments, the disease or disorder is MDS, AML, ovarian cancer, or non-small cell lung cancer. In certain embodiments, the ovarian cancer is epithelial ovarian cancer. In certain embodiments, the epithelial ovarian cancer is relapsed epithelial ovarian cancer. In certain embodiments, the disease or disorder is relapsed or refractory. In certain embodiments, the subject having a disease or disorder did not respond to a prior treatment. In certain embodiments, the prior treatment comprises an injectable hypomethylating agent. In certain embodiments, the prior treatment comprises a platinum based regimen. In a specific embodiment, the disease or disorder is MDS or AML that is not responding to prior treatment with an injectable hypomethylating agent. In another specific embodiment, the disease or disorder is EOC or non-small cell lung cancer that relapsed after treatment with a platinum based regimen.

In certain embodiments, the anti-PD1 monoclonal antibody is a humanized monoclonal IgG4 antibody. In one embodiment, the humanized monoclonal IgG4 antibody is pembrolizumab.

In certain embodiments, the anti-PDL1 monoclonal antibody is a humanized monoclonal IgG1 antibody. In one embodiment, the humanized monoclonal IgG1 antibody is durvalumab.

In one embodiment, the anti-PD1 monoclonal antibody is pembrolizumab, MK-3475, pidilizumab, nivolumab (BMS-936558, MDX-1106, or ONO-4538).

In one embodiment, the anti-PDL1 monoclonal antibody is BMS-936559, atezolizumab (MPDL3280A), or durvalumab (MEDI4736).

In one embodiment, the anti-PDL1 monoclonal antibody is durvalumab (MEDI4736).

In certain embodiments, the 5-azacytidine, or a pharmaceutically acceptable salt, solvate or hydrate thereof is administered for 21 consecutive days followed by seven consecutive days of rest in a 28 day cycle. In certain embodiments, the 5-azacytidine, or a pharmaceutically acceptable salt, solvate or hydrate thereof is administered for 14 consecutive days followed by seven consecutive days of rest in a 21 day cycle.

In certain embodiments, the anti-PD1/anti-PDL1 monoclonal antibody is administered on days 7 and 21 in a 28 day cycle. In certain embodiments, the anti-PD1/anti-PDL1 monoclonal antibody is administered on days 8 and 21 in a 28 day cycle. In certain embodiments, the anti-PD1/anti-PDL1 monoclonal antibody is administered on day 1 in a 28 day cycle. In certain embodiments, the anti-PD1/anti-PDL1 monoclonal antibody is administered on day 1 in a 21 day cycle.

In certain embodiments, the 5-azacytidine, or a pharmaceutically acceptable salt, solvate or hydrate thereof is administered for 21 consecutive days followed by seven consecutive days of rest in a 28 day cycle, and wherein the anti-PD1/anti-PDL1 monoclonal antibody is administered on days 7 and 21 of the 28 day cycle. In certain embodiments, the 5-azacytidine, or a pharmaceutically acceptable salt, solvate or hydrate thereof is administered for 21 consecutive days followed by seven consecutive days of rest in a 28 day cycle, and wherein the anti-PD1/anti-PDL1 monoclonal antibody is administered on days 8 and 21 of the 28 day cycle. In certain embodiments, the 5-azacytidine, or a pharmaceutically acceptable salt, solvate or hydrate thereof is administered for 21 consecutive days followed by seven consecutive days of rest in a 28 day cycle, and wherein the anti-PD1/anti-PDL1 monoclonal antibody is administered on day 1 of the 28 day cycle. In a specific embodiment, the disease or disorder is MDS or AML. In a more specific embodiment, the MDS or AML is relapsed or refractory. In a particular embodiment, the MDS or AML is not responding to treatment with injectable hypomethylating agents.

In certain embodiments, the 5-azacytidine, or a pharmaceutically acceptable salt, solvate or hydrate thereof is administered for 14 consecutive days followed by seven consecutive days of rest in a 21 day cycle, and wherein the anti-PD1/anti-PDL1 monoclonal antibody is administered on day 1 of the 21 day cycle. In a specific embodiment, the disease or disorder is ovarian cancer or lung cancer. In a more specific embodiment, the ovarian cancer is epithelial ovarian cancer, or relapsed or refractory epithelial ovarian cancer. In another more specific embodiment, the lung cancer is non-small cell lung cancer, or relapsed or refractory non-small cell lung cancer. In a particular embodiment, the epithelial ovarian cancer or the non-small cell lung cancer relapsed following a platinum based therapy.

In certain embodiments, the 5-azacytidine, or a pharmaceutically acceptable salt, solvate or hydrate thereof is administered in an amount of about 25 mg, about 50 mg, about 75 mg, about 100 mg, about 150 mg, about 200 mg, about 250 mg, about 300 mg, about 350 mg, about 400 mg, about 500 mg, or about 600 mg. In certain embodiments, the 5-azacytidine, or a pharmaceutically acceptable salt, solvate or hydrate thereof is administered in an amount of about 50 mg, about 100 mg, about 200 mg, about 300 mg, about 400 mg, about 500 mg, or about 600 mg per day.

In another embodiment, 5-azacytidine, or a pharmaceutically acceptable salt, solvate or hydrate thereof is administered in an amount of about 600 mg per day. In another embodiment, 5-azacytidine, or a pharmaceutically acceptable salt, solvate or hydrate thereof is administered in an amount of about 500 mg per day. In another embodiment, 5-azacytidine, or a pharmaceutically acceptable salt, solvate or hydrate thereof is administered in an amount of about 400 mg per day. In one embodiment, 5-azacytidine, or a pharmaceutically acceptable salt, solvate or hydrate thereof is administered in an amount of about 300 mg per day. In another embodiment, 5-azacytidine, or a pharmaceutically acceptable salt, solvate or hydrate thereof is administered in an amount of about 200 mg per day. In another embodiment, 5-azacytidine, or a pharmaceutically acceptable salt, solvate or hydrate thereof is administered in an amount of about 100 mg per day. In another embodiment, 5-azacytidine, or a pharmaceutically acceptable salt, solvate or hydrate thereof is administered in an amount of about 50 mg per day. In certain embodiments, 5-azacytidine, or a pharmaceutically acceptable salt, solvate or hydrate thereof, is administered once per day. In certain embodiments, 5-azacytidine, or a pharmaceutically acceptable salt, solvate or hydrate thereof, is administered twice per day. In one embodiment, 5-azacytidine or a pharmaceutically acceptable salt, solvate or hydrate thereof is administered in an amount of about 200 mg, about 150 mg, or about 100 mg twice per day. In one embodiment, 5-azacytidine or a pharmaceutically acceptable salt, solvate or hydrate thereof is administered in an amount of about 200 mg twice per day. In one embodiment, 5-azacytidine or a pharmaceutically acceptable salt, solvate or hydrate thereof is administered in an amount of about 150 mg twice per day. In one embodiment, 5-azacytidine or a pharmaceutically acceptable salt, solvate or hydrate thereof is administered in an amount of about 100 mg twice per day.

In certain embodiments, the anti-PD1/anti-PDL1 monoclonal antibody is administered parenterally. In certain embodiments, the anti-PD1/anti-PDL1 monoclonal antibody is administered in an amount of about 0.5 mg/Kg (about 0.5 mg of anti-PD1/anti-PDL1 monoclonal antibody per kilogram of a subject's mass), about 1 mg/Kg, about 2 mg/Kg, about 3 mg/Kg, about 4 mg/Kg, about 5 mg/Kg, about 6 mg/Kg, about 7 mg/Kg, about 8 mg/Kg, about 9 mg/Kg, about 10 mg/Kg, about 11 mg/Kg, about 12 mg/Kg, about 13 mg/Kg, about 14 mg/Kg, about 15 mg/Kg, about 16 mg/Kg, about 17 mg/Kg, about 18 mg/Kg, about 19 mg/Kg, or about 20 mg/Kg. In a specific embodiment, the anti-PD1/anti-PDL1 monoclonal antibody is administered intravenously in an amount of about 20 mg/Kg per day. In a specific embodiment, the anti-PD1/anti-PDL1 monoclonal antibody is administered intravenously in an amount of about 19 mg/Kg per day. In a specific embodiment, the anti-PD1/anti-PDL1 monoclonal antibody is administered intravenously in an amount of about 18 mg/Kg per day. In a specific embodiment, the anti-PD1/anti-PDL1 monoclonal antibody is administered intravenously in an amount of about 17 mg/Kg per day. In a specific embodiment, the anti-PD1/anti-PDL1 monoclonal antibody is administered intravenously in an amount of about 16 mg/Kg per day. In a specific embodiment, the anti-PD1/anti-PDL1 monoclonal antibody is administered intravenously in an amount of about 15 mg/Kg per day. In a specific embodiment, the anti-PD1/anti-PDL1 monoclonal antibody is administered intravenously in an amount of about 14 mg/Kg per day. In a specific embodiment, the anti-PD1/anti-PDL1 monoclonal antibody is administered intravenously in an amount of about 13 mg/Kg per day. In a specific embodiment, the anti-PD1/anti-PDL1 monoclonal antibody is administered intravenously in an amount of about 12 mg/Kg per day. In a specific embodiment, the anti-PD1/anti-PDL1 monoclonal antibody is administered intravenously in an amount of about 11 mg/Kg per day. In a specific embodiment, the anti-PD1/anti-PDL1 monoclonal antibody is administered intravenously in an amount of about 10 mg/Kg per day. In a specific embodiment, the anti-PD1/anti-PDL1 monoclonal antibody is administered intravenously in an amount of about 9 mg/Kg per day. In a specific embodiment, the anti-PD1/anti-PDL1 monoclonal antibody is administered intravenously in an amount of about 8 mg/Kg per day. In a specific embodiment, the anti-PD1/anti-PDL1 monoclonal antibody is administered intravenously in an amount of about 7 mg/Kg per day. In a specific embodiment, the anti-PD1/anti-PDL1 monoclonal antibody is administered intravenously in an amount of about 6 mg/Kg per day. In a specific embodiment, the anti-PD1 or anti-PDL1 monoclonal antibody is administered intravenously in an amount of about 5 mg/Kg. In a specific embodiment, the anti-PD1/anti-PDL1 monoclonal antibody is administered intravenously in an amount of about 4 mg/Kg per day. In a specific embodiment, the anti-PD1/anti-PDL1 monoclonal antibody is administered intravenously in an amount of about 3 mg/Kg per day. In a specific embodiment, the anti-PD1/anti-PDL1 monoclonal antibody is administered intravenously in an amount of about 2 mg/Kg per day. In a specific embodiment, the anti-PD1/anti-PDL1 monoclonal antibody is administered intravenously in an amount of about 1 mg/Kg per day. In a specific embodiment, the anti-PD1/anti-PDL1 monoclonal antibody is administered intravenously in an amount of about 0.5 mg/Kg per day. In a specific embodiment, the anti-PD 1 or anti-PDL1 monoclonal antibody is administered intravenously in an amount of about 1,500 mg per day. In a specific embodiment, the anti-PD1 or anti-PDL1 monoclonal antibody is administered in an amount of about 1,500 mg per day on day 1 in a 28 day cycle. In a particular embodiment, the anti-PD1/anti-PDL1 monoclonal antibody is administered intravenously in an amount of about 10 mg/Kg per day on day 1 in a 28 day cycle, days 7 and 21 in a 28 day cycle, or on days 8 and 21 in a 28 day cycle. In a particular embodiment, the anti-PD1/anti-PDL1 monoclonal antibody is administered intravenously in an amount of about 10 mg/Kg per day on day 1 in a 28 day cycle. In a particular embodiment, the anti-PD1/anti-PDL1 monoclonal antibody is administered intravenously in an amount of about 10 mg/Kg per day on days 7 and 21 in a 28 day cycle. In a particular embodiment, the anti-PD1/anti-PDL1 monoclonal antibody is administered intravenously in an amount of about 10 mg/Kg per day on days 8 and 21 in a 28 day cycle. In a particular embodiment, the anti-PD1/anti-PDL1 monoclonal antibody is administered intravenously in an amount of about 5 mg/Kg per day on days 7 and 21 in a 28 day cycle. In one embodiment, the anti-PD1 monoclonal antibody is pembrolizumab and is administered as a 30 minute i.v. infusion.

In one embodiment, the anti-PD1 monoclonal antibody is MK-3475 and is administered as a 30 minute i.v. infusion.

In one embodiment, the anti-PD1 monoclonal antibody is pidilizumab and is administered as a 30 minute i.v. infusion.

In one embodiment, the anti-PD1 monoclonal antibody is nivolumab (BMS-936558, MDX-1106, or ONO-4538) and is administered as a 30 minute i.v. infusion.

In one embodiment, the anti-PDL1 monoclonal antibody is atezolizumab (MPDL3280A) and is administered as a 30 minute i.v. infusion.

In one embodiment, the anti-PDL1 monoclonal antibody is durvalumab (MEDI4736) and is administered as a 30 minute i.v. infusion. In one embodiment, durvalumab (MEDI4736) is administered on Day 1 of each 28-day treatment cycle as a single 1500 mg IV infusion.

In certain embodiments, 5-azacytidine or a pharmaceutically acceptable salt, solvate or hydrate thereof is in a form of a capsule, tablet or caplet.

In certain embodiments, the method further comprises administering a therapeutically effective amount of an additional active agent.

In a specific embodiment, the subject is a human.

In certain embodiments, the cytidine analog is 5-azacytidine. In other embodiments, the cytidine analog is 5-aza-2′-deoxycytidine (decitabine or 5-aza-CdR). In yet other embodiments, the cytidine analog is, for example: 1-β-D-arabinofuranosylcytosine (Cytarabine or ara-C); pseudoisocytidine (psi ICR); 5-fluoro-2′-deoxycytidine (FCdR); 2′-deoxy-2′,2′-difluorocytidine (Gemcitabine); 5-aza-2′-deoxy-2′,2′-difluorocytidine; 5-aza-2′-deoxy-2′-fluorocytidine; l-j-D-ribofuranosyl-2(1H)-pyrimidinone (Zebularine); 2′,3′-dideoxy-5-fluoro-3′-thiacytidine (Emtriva); 2′-cyclocytidine (Ancitabine); 1-β-D-arabinofuranosyl-5-azacytosine (Fazarabine or ara-AC); 6-azacytidine (6-aza-CR); 5,6-dihydro-5-azacytidine (dH-aza-CR); N4 pentyloxycarbonyl-5′-deoxy-5-fluorocytidine (Capecitabine); N4 octadecyl-cytarabine; elaidic acid cytarabine; or their derivatives or related analogs.

Certain embodiments herein provide compositions that are single unit dosage forms comprising a cytidine analog. Certain embodiments herein provide compositions that are non-enteric-coated. Certain embodiments herein provide compositions that are tablets comprising a non-enteric coating. Certain embodiments herein provide compositions that are tablets comprising a cytidine analog. Certain embodiments herein provide compositions that are capsules comprising a cytidine analog. In certain embodiments, the single unit dosage forms optionally further contain one or more excipients. In certain embodiments, the tablets optionally further contain one or more excipients. In other embodiments, the capsules optionally further contain one or more excipients. In certain embodiments, the composition is a tablet that effects an immediate release of the API upon oral administration. In other embodiments, the composition is a tablet that effects a controlled release of the API substantially in the stomach. In other embodiments, the composition is a tablet that effects a controlled release of the API substantially in the stomach and the upper intestine. In certain embodiments, the composition is a capsule that effects an immediate release of the API upon oral administration. In other embodiments, the composition is a capsule that effects a controlled release of the API substantially in the stomach. In other embodiments, the composition is a capsule that effects a controlled release of the API substantially in the stomach and the upper intestine. In particular embodiments, the tablet contains a drug core that comprises a cytidine analog, and optionally further contains a coating of the drug core, wherein the coating is applied to the drug core using an aqueous solvent, such as, for example, water, or non-aqueous solvent, such as, for example ethanol.

Certain embodiments herein provide methods of using the formulations provided herein to treat, prevent, or manage diseases or disorders including, e.g., cancer, disorders related to abnormal cell proliferation, solid tumors, hematologic disorders, or immune disorders. In certain embodiments, the formulations of cytidine analogs are orally administered to subjects in need thereof to treat, prevent, or manage a cancer; or a hematological disorder, such as, for example, MDS, AML, ALL, CML, NHL, leukemia, lymphoma, or multiple myeloma; or a solid tumor, such as, for example, sarcoma, melanoma, carcinoma, or cancer of the colon, breast, ovary, gastrointestinal system, kidney, bladder, lung (e.g., non-small-cell lung cancer and small-cell lung cancer), testicle, prostate, stomach, pancreas, liver, head and neck, brain, skin, or bone, among others. In particular embodiments, the cancer is a cancer of the bladder, ovary, pancreas, lung, colon, head and neck, breast, or skin. In particular embodiments, the cancer is a cancer of the bladder, ovary, pancreas, lung, or colon. In certain embodiments, the cancer is refractory. In certain embodiments, the cancer is relapsed. In certain embodiments, the cancer is metastatic. In certain embodiments, the formulations of cytidine analogs are orally administered to subjects in need thereof to treat, prevent, or manage an immune disorder. In certain embodiments, the oral formulations provided herein are co-administered with one or more therapeutic agents to provide a synergistic therapeutic effect in subjects in need thereof. In certain embodiments, the oral formulations provided herein are co-administered with one or more therapeutic agents to provide a resensitization effect in subjects in need thereof. The co-administered agents may be a cancer therapeutic agent, as described herein. In certain embodiments, the co-administered agent(s) may be dosed, e.g., orally or by injection. In certain embodiments, the cytidine and/or the co-administered agent(s) may be dosed cyclically.

In certain embodiments, provided herein are tablets containing 5-azacytidine and methods for making and using the tablets to treat cancer, disorders related to abnormal cell proliferation, or hematologic disorders. In certain embodiments, the tablets optionally further contain one or more excipients such as, for example, glidants, diluents, lubricants, colorants, disintegrants, granulating agents, binding agents, polymers, and/or coating agents. Examples of ingredients useful in preparing certain formulations provided herein are described in, e.g., Etter et al., U.S. Patent Application Publication No. 2008/0057086 (application Ser. No. 11/849,958), which is incorporated herein by reference in its entirety.

In certain embodiments provided herein are methods of using the formulations provided herein to treat, prevent, or manage diseases or disorders including, e.g., non-small lung cancer (NSCLC) by administering one or more cytidine analogs to subjects having NSCLC. In certain embodiments, the methods comprise treating, preventing or managing certain types of NSCLC, including but not limited to, epidermoid or squamous cell carcinoma, large cell carcinoma, adenocarcinoma, adenosquamous carcinoma, carcinomas with pleomorphic, sarcomatoid or sarcomatous elements, carcinoid tumor, carcinomas of salivary-gland, and unclassified carcinoma. In certain embodiments, the methods comprise treating, preventing or managing certain stages of NSCLC, including but not limited to, occult carcinoma, Stage 0, Stage IA, Stage IB, Stage IIA, Stage IIB, Stage IIIA, Stage IIIB, and Stage IV, in a subject having NSCLC. In certain embodiments, the methods comprise treating or managing NSCLC in a subject having particular NSCLC cell types. In certain embodiments, the cytidine analog is 5-azacytidine (azacitidine). In certain embodiments, the cytidine analog is 5-aza-2′-deoxycytidine (decitabine). In certain embodiments, the methods comprise co-administering two or more active agents. In certain embodiments, the active agent is an anti-PD1/anti-PDL1 monoclonal antibody. In certain embodiments, the active agent is pembrolizumab, MK-3475, pidilizumab, Nivolumab (BMS-936558, MDX-1106, or ONO-4538). In certain embodiments, the active agent is BMS-936559, atezolizumab (MPDL3280A), or durvalumab (MEDI4736). In certain embodiments, the methods comprise treating, preventing or managing NSCLC using one or more of the methods provided herein, together with one or more of the treatments selected from surgery, chemotherapy, immunotherapy, targeted therapy, and radiation therapy.

Certain embodiments herein provide methods of treating certain NSCLC cell types, including but not limited to, A549, H1975, H23, H460, and/or H1299, in a subject having NSCLC. Particular methods comprise identifying the presence of at least one NSCLC cell type, including but not limited to, A549, H1975, H23, H460, and/or H1299, in a subject having NSCLC. Particular methods comprise administering one or more cytidine analogs to a subject having NSCLC to treat one or more NSCLC cell types, including but not limited to, A549, H1975, H23, H460, and/or H1299. Particular methods comprise administering 5-azacytidine to a subject having NSCLC to treat one or more NSCLC cell types, including but not limited to, A549, H1975, H23, H460, and/or H1299. Particular embodiments herein provide methods for treating a subject with NSCLC by administering 5-azacytidine to the subject, wherein the NSCLC includes a cell type selected from A549, H1975, H23, H460, and H1299.

Certain embodiments herein provide methods of treating, preventing or managing certain types of NSCLC, including but not limited to, (1) squamous cell carcinoma, including but not limited to, papillary, clear cell, small cell, and basaloid carcinoma; (2) adenocarcinoma, including but not limited to, acinar, papillary, bronchioloalveolar carcinoma (nonmucinous, mucinous, mixed mucinous and nonmucinous or indeterminate cell type), solid adenocarcinoma with mucin, adenocarcinoma with mixed subtypes, and other variants including well-differentiated fetal adenocarcinoma, mucinous (colloid) adenocarcinoma, mucinous cystadenocarcinoma, signet ring adenocarcinoma, and clear cell adenocarcinoma; (3) large cell carcinoma, including but not limited to, large cell neuroendocrine carcinoma, combined large cell neuroendocrine carcinoma, basaloid carcinoma, lymphoepithelioma-like carcinoma, clear cell carcinoma, and large cell carcinoma with rhabdoid phenotype; (4) adenosquamous carcinoma; (5) carcinomas with pleomorphic, sarcomatoid, or sarcomatous elements, including but not limited to, carcinomas with spindle and/or giant cells, spindle cell carcinoma, giant cell carcinoma, carcinosarcoma, and pulmonary blastoma; (6) carcinoid tumor, including but not limited to, typical carcinoid and atypical carcinoid; (7) carcinomas of salivary-gland, including but not limited to, mucoepidermoid carcinoma and adenoid cystic carcinoma; and (8) unclassified carcinoma. Particular embodiments herein provide methods of treating, preventing or managing NSCLC in the primary tumor, lymph nodes, and/or distant metastasis, in a subject having NSCLC. Particular embodiments herein provide methods of treating NSCLC in a subject having surgically resectable NSCLC, locally or regionally advanced NSCLC, and/or distant metastatic NSCLC.

Certain embodiments herein provide, inter alia, pharmaceutical compositions comprising a therapeutically effective amount of 5-azacytidine, wherein the composition releases the 5-azacytidine substantially in the stomach following oral administration to a subject. Further embodiments provide the aforementioned compositions, which: are immediate release compositions; do not have an enteric coating (i.e., are non-enteric-coated); are tablets; are capsules; further comprise an excipient selected from any excipient disclosed herein; further comprise a permeation enhancer; further comprise d-alpha-tocopheryl polyethylene glycol 1000 succinate; further comprise a permeation enhancer in the formulation at about 2% by weight relative to the total weight of the formulation; are essentially free of a cytidine deaminase inhibitor; are essentially free of tetrahydrouridine; have an amount of 5-azacytidine of at least about 40 mg; have an amount of 5-azacytidine of at least about 400 mg; have an amount of 5-azacytidine of at least about 1000 mg; achieve an area-under-the-curve value of at least about 200 ng-hr/mL following oral administration to a subject; achieve an area-under-the-curve value of at least about 400 ng-hr/mL following oral administration to a subject; achieve a maximum plasma concentration of at least about 100 ng/mL following oral administration to a subject; achieve a maximum plasma concentration of at least about 200 ng/mL following oral administration to a subject; achieve a time to maximum plasma concentration of less than about 90 minutes following oral administration to a subject; and/or achieve a time to maximum plasma concentration of less than about 60 minutes following oral administration to a subject.

Certain embodiments herein provide a pharmaceutical composition for oral administration comprising a therapeutically effective amount of 5-azacytidine, which releases the 5-azacytidine substantially in the stomach and achieves an area-under-the-curve value of at least about 200 ng-hr/mL following oral administration.

Certain embodiments herein provide a pharmaceutical composition for oral administration comprising a therapeutically effective amount of 5-azacytidine, which releases the 5-azacytidine substantially in the stomach and achieves an area-under-the-curve value of at least about 400 ng-hr/mL following oral administration.

Certain embodiments herein provide a pharmaceutical composition for oral administration comprising a therapeutically effective amount of 5-azacytidine, which releases the 5-azacytidine substantially in the stomach and achieves a maximum plasma concentration of at least about 100 ng/mL following oral administration.

Certain embodiments herein provide a pharmaceutical composition for oral administration comprising a therapeutically effective amount of 5-azacytidine, which releases the 5-azacytidine substantially in the stomach and achieves a maximum plasma concentration of at least about 200 ng/mL following oral administration.

Certain embodiments herein provide a pharmaceutical composition for oral administration comprising a therapeutically effective amount of 5-azacytidine, which releases the 5-azacytidine substantially in the stomach and achieves a time to maximum plasma concentration of, e.g., less than about 6 hr, less than about 5 hr, less than about 4 hr, less than about 3 hr, less than about 2.5 hr. less than about 2 hr, less than about 1.5 hr, less than about 1 hr, less than about 45 min, or less than about 30 min following oral administration. In specific embodiments, the presence of food may affect (e.g., extend) the total exposure and/or time to maximum plasma concentration.

Certain embodiments herein provide a pharmaceutical composition for oral administration comprising a therapeutically effective amount of 5-azacytidine, which releases the 5-azacytidine substantially in the stomach and achieves a time to maximum plasma concentration of less than about 60 minutes following oral administration.

Certain embodiments herein provide any of the aforementioned compositions, as single unit dosage forms, tablets, or capsules.

Certain embodiments herein provide, inter alia, methods for treating a subject having a disease associated with abnormal cell proliferation, comprising orally administering to the subject a pharmaceutical composition comprising a therapeutically effective amount of 5-azacytidine, wherein the composition releases the 5-azacytidine substantially in the stomach following oral administration to the subject. Further embodiments herein provide the aforementioned methods, in which: the disease is myelodysplastic syndrome; the disease is acute myelogenous leukemia; the method further comprises co-administering to the subject in need thereof an additional therapeutic agent selected from any additional therapeutic agent disclosed herein; the composition is an immediate release composition; the composition does not have an enteric coating; the composition further comprises a permeation enhancer the composition further comprises the permeation enhancer d-alpha-tocopheryl polyethylene glycol 1000 succinate; the composition further comprises d-alpha-tocopheryl polyethylene glycol 1000 succinate in the formulation at about 2% by weight relative to the total weight of the formulation; the method further comprises not co-administering a cytidine deaminase inhibitor with the cytidine analog; the composition is a single unit dosage form; the composition is a tablet; the composition is a capsule; the composition further comprises an excipient selected from any excipient disclosed herein; the amount of 5-azacytidine is at least about 40 mg; the amount of 5-azacytidine is at least about 400 mg; the amount of 5-azacytidine is at least about 1000 mg; the method achieves an area-under-the-curve value of at least about 200 ng-hr/mL following oral administration to the subject; the method achieves an area-under-the-curve value of at least about 400 ng-hr/mL following oral administration to the subject; the method achieves a maximum plasma concentration of at least about 100 ng/mL following oral administration to the subject; the method achieves a maximum plasma concentration of at least about 200 ng/mL following oral administration to the subject; the method achieves a time to maximum plasma concentration of less than about 90 minutes following oral administration to the subject; and/or the method achieves a time to maximum plasma concentration of less than about 60 minutes following oral administration to the subject

Certain embodiments herein provide, inter alia, pharmaceutical compositions comprising a therapeutically effective amount of 5-azacytidine, wherein the compositions are for treating a disease or disorder associated with abnormal cell proliferation, wherein the compositions are prepared for oral administration, and wherein the compositions are prepared for release of the 5-azacytidine substantially in the stomach. Further embodiments herein provide the aforementioned compositions, which: have an amount of 5-azacytidine of about 40 mg, about 400 mg, or about 1000 mg; are prepared to achieve an area-under-the-curve value of at least about 200 ng-hr/mL or 400 ng-hr/mL following oral administration; are prepared to achieve a maximum plasma concentration of at least about 100 ng/mL or 200 ng/mL following oral administration; are prepared to achieve a time to maximum plasma concentration of less than about 60 minutes or 90 minutes after being administered; are prepared in the form of an immediate release composition; are prepared for oral administration in combination with an additional therapeutic agent selected from any additional therapeutic agent disclosed herein; are for treating myelodysplastic syndrome or acute myelogenous leukemia; further comprise a permeation enhancer; which further comprise the permeation enhancer d-alpha-tocopheryl polyethylene glycol 1000 succinate; are single unit dosage forms; are tablets or capsules; and/or further comprise an excipient selected from any excipient disclosed herein.

Certain embodiments herein provide, inter alia, uses of 5-azacytidine for the preparation of a pharmaceutical composition for treating a disease associated with abnormal cell proliferation, wherein the composition is prepared for oral administration, and wherein the composition is prepared for release of the 5-azacytidine substantially in the stomach. Further embodiments herein provide the aforementioned uses, in which: the disease is myelodysplastic syndrome or acute myelogenous leukemia; the amount of 5-azacytidine is selected from any amount disclosed herein; and/or the composition is prepared for immediate release. Further embodiments provide, inter alia, methods for treating a subject having a disease or disorder provided herein by administering a pharmaceutical compositions provided herein, wherein the treatment results in improved survival of the subject.

### IV. BRIEF DESCRIPTION OF THE DRAWINGS

FIG. 1 depicts oral 5-azacytidine/pembrolizumab study flow.

FIG. 2 depicts a dosing regimen.

FIG. 3 depicts a flow diagram of a phase I design.

FIG. 4 depicts a flow diagram of a phase II design.

FIG. 5 depicts a flow diagram of next steps of study.

FIG. 6 depicts Safety run-in phase study design.

FIG. 7 depicts treatment phase study design.

### V. DETAILED DESCRIPTION

Unless defined otherwise, all technical and scientific terms used herein have the same meaning as commonly understood by one of ordinary skill in the art. All publications and patents referred to herein are incorporated by reference herein in their entireties.

### A. Definitions

As used in the specification and the accompanying claims, the indefinite articles “a” and “an” and the definite article “the” include plural as well as singular referents, unless the context clearly dictates otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range.

As used herein, and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a subject with such a disease or disorder. In some embodiments, the terms refer to the administration of a compound or dosage form provided herein, with or without one or more additional active agent(s), after the onset of symptoms of the particular disease.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. Subjects with familial history of a disease in particular are candidates for preventive regimens in certain embodiments. In addition, subjects who have a history of recurring symptoms are also potential candidates for prevention. In this regard, the term “prevention” may be interchangeably used with the term “prophylactic treatment.”

As used herein, and unless otherwise specified, the terms “manage,” “managing” and “management” refer to preventing or slowing the progression, spread or worsening of a disease or disorder, or of one or more symptoms thereof. Often, the beneficial effects that a subject derives from a prophylactic and/or therapeutic agent do not result in a cure of the disease or disorder. In this regard, the term “managing” encompasses treating a subject who had suffered from the particular disease in an attempt to prevent or minimize the recurrence of the disease.

As used herein, amelioration of the symptoms of a particular disorder by administration of a particular pharmaceutical composition refers to any lessening, whether permanent or temporary, lasting or transient, that can be attributed to or associated with administration of the composition.

As used herein, and unless otherwise specified, the terms “therapeutically effective amount” and “effective amount” of a compound mean an amount sufficient to provide a therapeutic benefit in the treatment or management of a disease or disorder, or to delay or minimize one or more symptoms associated with the disease or disorder. A “therapeutically effective amount” and “effective amount” of a compound mean an amount of therapeutic agent, alone or in combination with one or more other agent(s), which provides a therapeutic benefit in the treatment or management of the disease or disorder. The terms “therapeutically effective amount” and “effective amount” can encompass an amount that improves overall therapy, reduces or avoids symptoms or causes of disease or disorder, or enhances the therapeutic efficacy of another therapeutic agent.

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

“Tumor,” as used herein, refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth.

The terms “cancer” and “cancerous” refer to or describe the physiological condition in mammals that is typically characterized by unregulated cell growth. Examples of cancer include, but are not limited to blood borne (e.g., lymphoma, leukemia) and solid tumors.

As used herein, and unless otherwise specified, the term “proliferative” disorder or disease refers to unwanted cell proliferation of one or more subset of cells in a multicellular organism resulting in harm (i.e., discomfort or decreased life expectancy) to the multicellular organism. For example, as used herein, proliferative disorder or disease includes neoplastic disorders and other proliferative disorders.

As used herein, and unless otherwise specified, the term “relapsed” refers to a situation where a subject, that has had a remission of cancer after a therapy, has a return of cancer cells.

As used herein, and unless otherwise specified, the term “refractory” or “resistant” refers to a circumstance where a subject, even after intensive treatment, has residual cancer cells in the body.

The terms “composition,” “formulation,” and “dosage form,” as used herein are intended to encompass compositions comprising the specified ingredient(s) (in the specified amounts, if indicated), as well as any product(s) which result, directly or indirectly, from combination of the specified ingredient(s) in the specified amount(s). By “pharmaceutical” or “pharmaceutically acceptable” it is meant that any diluent(s), excipient(s) or carrier(s) in the composition, formulation, or dosage form are compatible with the other ingredient(s) and not deleterious to the recipient thereof. Unless indicated otherwise, the terms “composition,” “formulation,” and “dosage form” are used herein interchangeably.

The term “immediate release,” when used herein in reference to a composition, formulation, or dosage form provided herein, means that the composition, formulation, or dosage form does not comprise a component (e.g., a coating) that serves to delay the spatial and/or temporal release of some or all of the API from the composition, formulation, or dosage form beyond the stomach following oral administration. In certain embodiments, an immediate release composition, formulation, or dosage form is one that releases the API substantially in the stomach following oral administration. In specific embodiments, an immediate release composition, formulation, or dosage form is one that is not delayed-release. In specific embodiments, an immediate release composition, formulation, or dosage form is one that does not comprise an enteric coating.

The terms “non-enteric-coated” and “non-enteric coating,” when used herein, refers to a pharmaceutical composition, formulation, or dosage form that does not comprise a coating intended to release the active ingredient(s) beyond the stomach (e.g., in the intestine). In certain embodiments, a non-enteric-coated composition, formulation, or dosage form is designed to release the active ingredient(s) substantially in the stomach.

The term “substantially in the stomach,” when used herein in reference to a composition, formulation, or dosage form provided herein, means that at least about 99%, at least about 95%, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, or at least about 10% of the cytidine analog is released in the stomach. The term “released in the stomach” and related terms as used herein refer to the process whereby the cytidine analog is made available for uptake by or transport across cells lining the stomach and then made available to the body.

The term “subject” is defined herein to include animals such as mammals, including, but not limited to, primates (e.g., humans), cows, sheep, goats, horses, dogs, cats, rabbits, rats, mice and the like. In specific embodiments, the subject is a human.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

The term “solvate” refers to a complex or aggregate formed by one or more molecules of a solute, e.g., a compound provided herein, and one or more molecules of a solvent, which present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The term “isotopic composition” refers to the amount of each isotope present in a given atomic position, and “natural isotopic composition” refers to the naturally occurring isotopic composition or abundance for a given atomic position. Atomic positions containing their natural isotopic composition may also be referred to herein as “non-enriched.” Unless otherwise designated, the atomic positions of the compounds recited herein are meant to represent any stable isotope of that atom. For example, unless otherwise stated, when a position is designated specifically as “H” or “hydrogen,” the position is understood to have hydrogen at its natural isotopic composition.

The term “isotopically enriched” refers to an atomic position having an isotopic composition other than the natural isotopic composition of that atom. “Isotopically enriched” may also refer to a compound containing at least one atomic position having an isotopic composition other than the natural isotopic composition of that atom. As used herein, an “isotopologue” is an isotopically enriched compound.

The term “isotopic enrichment” refers to the percentage of incorporation of an amount of a specific isotope at a given atomic position in a molecule in the place of that atom's natural isotopic composition. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%.

The term “isotopic enrichment factor” refers to the ratio between the isotopic composition and the natural isotopic composition of a specified isotope.

With regard to the compounds provided herein, when a particular atomic position is designated as having deuterium or “D,” it is understood that the abundance of deuterium at that position is substantially greater than the natural abundance of deuterium, which is about 0.015%. A position designated as having deuterium typically has a minimum isotopic enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium position.

The isotopic enrichment and isotopic enrichment factor of the compounds provided herein can be determined using conventional analytical methods known to one of ordinary skill in the art, including, e.g., mass spectrometry, nuclear magnetic resonance spectroscopy, and crystallography.

### B. Cytidine Analogs

1. Overview

Provided herein are dosage forms, pharmaceutical formulations and compositions comprising cytidine analogs that release the API substantially in the stomach upon oral administration. In certain embodiments, the cytidine analog is 5-azacytidine. In certain embodiments, the cytidine analog is 5-aza-2′-deoxycytidine (decitabine or 5-aza-CdR). In certain embodiments, the cytidine analog is, for example: 1-β-D-arabinofuranosylcytosine (Cytarabine or ara-C); pseudoiso-cytidine (psi ICR); 5-fluoro-2′-deoxycytidine (FCdR); 2′-deoxy-2′,2′-difluorocytidine (Gemcitabine); 5-aza-2′-deoxy-2′,2′-difluorocytidine; 5-aza-2′-deoxy-2′-fluorocytidine; 1-β-D-ribofuranosyl-2(1H)-pyrimidinone (Zebularine); 2′,3′-dideoxy-5-fluoro-3′-thiacytidine (Emtriva); 2′-cyclocytidine (Ancitabine); 1-β-D-arabinofuranosyl-5-azacytosine (Fazarabine or ara-AC); 6-azacytidine (6-aza-CR); 5,6-dihydro-5-azacytidine (dH-aza-CR); N⁴-pentyloxy-carbonyl-5′-deoxy-5-fluorocytidine (Capecitabine); N⁴-octadecyl-cytarabine; elaidic acid cytarabine; or a conjugated compound comprising a cytidine analog and a fatty acid (e.g., an azacitidine-fatty acid conjugate, including, but not limited to, CP-4200 (Clavis Pharma ASA) or a compound disclosed in WO 2009/042767, such as aza-C-5′-petroselinic acid ester or aza-C-5′-petroselaidic acid ester).

In certain embodiments, cytidine analogs provided herein include esterified derivatives of cytidine analogs, such as, e.g., esterified derivatives of 5-azacytidine. In particular embodiments, esterified derivatives are cytidine analogs that contain an ester moiety (e.g., an acetyl group) at one or more positions on the cytidine analog molecule. Esterified derivatives may be prepared by any method known in the art. In certain embodiments, esterified derivatives of a cytidine analog serve as prodrugs of the cytidine analog, such that, e.g., following administration of an esterified derivative, the derivative is deacetylated in vivo to yield the cytidine analog. A particular embodiment herein provides 2′,3′,5′-triacetyl-5-azacytidine (TAC), which possesses favorable physical-chemical and therapeutic properties. See, e.g., International Publication No. WO 2008/092127 (International Application No. PCT/US2008/052124); Ziemba, A. J., et al., “Development of Oral Demethylating Agents for the Treatment of Myelodysplastic Syndrome” (Abstract No. 3369), In: Proceedings of the 100th Annual Meeting of the American Association for Cancer Research; 2009 Apr. 18-22; Denver, Co. Philadelphia (Pa.): AACR; 2009 (both of which are incorporated by reference herein in their entireties).

In certain embodiments, the cytidine analogs provided herein include any compound which is structurally related to cytidine or deoxycytidine and functionally mimics and/or antagonizes the action of cytidine or deoxycytidine. Certain embodiments herein provide salts, cocrystals, solvates (e.g., hydrates), complexes, prodrugs, precursors, metabolites, and/or other derivatives of the cytidine analogs provided herein. For example, particular embodiments provide salts, cocrystals, solvates (e.g., hydrates), complexes, precursors, metabolites, and/or other derivatives of 5-azacytidine. Certain embodiments provide cytidine analogs that are not salts, cocrystals, solvates (e.g., hydrates), or complexes of the cytidine analogs provided herein. For example, particular embodiments provide 5-azacytidine in a non-ionized, non-solvated (e.g., anhydrous), non-complexed form. Certain embodiments herein provide mixtures of two or more cytidine analogs provided herein.

Cytidine analogs provided herein may be prepared using synthetic methods and procedures referenced herein or otherwise available in the literature. For example, particular methods for synthesizing 5-azacytidine are taught in, e.g., U.S. Pat. No. 7,038,038 and references discussed therein, each of which is incorporated herein by reference. 5-Azacytidine is also available from Celgene Corporation, Warren, N.J. Other cytidine analogs provided herein may be prepared using previously disclosed synthetic procedures available to a person of ordinary skill in the art.

In certain embodiments, exemplary cytidine analogs have the structures provided below:

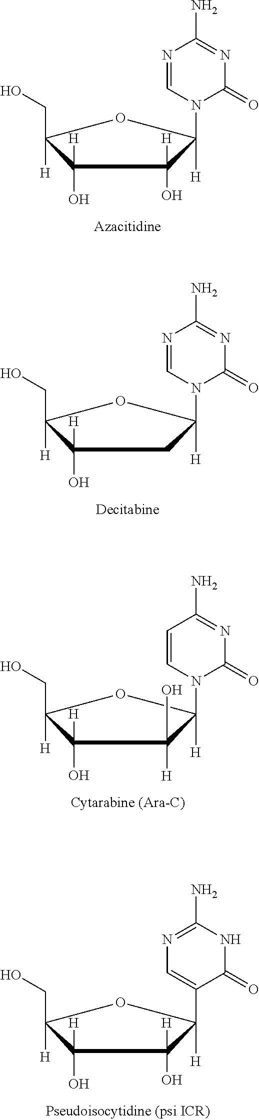

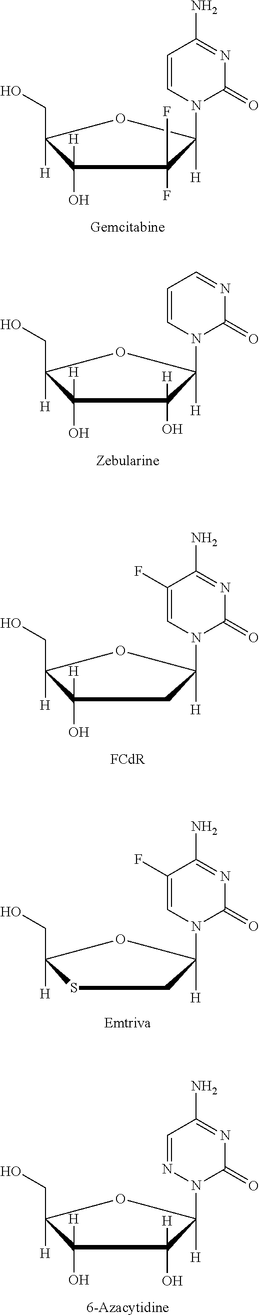

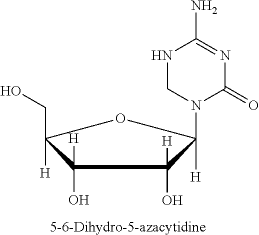

2. Isotopically Enriched Cytidine Analogs

Particular embodiments herein provide isotopically enriched cytidine analogs, prodrugs thereof, synthetic intermediates thereof, and metabolites thereof. For example, specific embodiments herein provide isotopically enriched 5-azacytidine.

Isotopic enrichment (e.g., deuteration) of pharmaceuticals to improve pharmacokinetics (“PK”), pharmacodynamics (“PD”), and toxicity profiles, has been demonstrated previously with some classes of drugs. See, e.g., Lijinsky et. al., Food Cosmet. Toxicol., 20: 393 (1982); Lijinsky et. al., J. Nat. Cancer Inst., 69: 1127 (1982); Mangold et. al., Mutation Res. 308: 33 (1994); Gordon et. al., Drug Metab. Dispos., 15: 589 (1987); Zello et. al., Metabolism, 43: 487 (1994); Gately et. al., J. Nucl. Med., 27: 388 (1986); Wade, D., Chem. Biol. Interact. 117: 191 (1999).

Without being limited by any particular theory, isotopic enrichment of a drug can be used, for example, to: (1) reduce or eliminate unwanted metabolites; (2) increase the half-life of the parent drug; (3) decrease the number of doses needed to achieve a desired effect; (4) decrease the amount of a dose necessary to achieve a desired effect; (5) increase the formation of active metabolites, if any are formed; and/or (6) decrease the production of deleterious metabolites in specific tissues and/or create a more effective drug and/or a safer drug for combination therapy, whether the combination therapy is intentional or not.

Replacement of an atom for one of its isotopes may often result in a change in the reaction rate of a chemical reaction. This phenomenon is known as the Kinetic Isotope Effect (“KIE”). For example, if a C—H bond is broken during a rate-determining step in a chemical reaction (i.e. the step with the highest transition state energy), substitution of a deuterium for that hydrogen will cause a decrease in the reaction rate and the process will slow down. This phenomenon is known as the Deuterium Kinetic Isotope Effect (“DKIE”). See, e.g. Foster et al., Adv. Drug Res., vol. 14, pp. 1-36 (1985); Kushner et al., Can. J. Physiol. Pharmacol., vol. 77, pp. 79-88 (1999).

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. Without being limited by a particular theory, high DKIE values may be due in part to a phenomenon known as tunneling, which is a consequence of the uncertainty principle. Tunneling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon.

Tritium (“T”) is a radioactive isotope of hydrogen, used in research, fusion reactors, neutron generators and radiopharmaceuticals. Tritium is a hydrogen atom that has 2 neutrons in the nucleus and has an atomic weight close to 3. It occurs naturally in the environment in very low concentrations, most commonly found as T₂O. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects.

Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, may lead to an analogous kinetic isotope effect.

The animal body expresses a variety of enzymes for the purpose of eliminating foreign substances, such as therapeutic agents, from its circulation system. Examples of such enzymes include the cytochrome P450 enzymes (“CYPs”), esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) pi-bond. The resultant metabolites may be stable or unstable under physiological conditions, and can have substantially different pharmacokinetic, pharmacodynamic, and acute and long-term toxicity profiles relative to the parent compounds. For many drugs, such oxidations are rapid. As a result, these drugs often require the administration of multiple or high daily doses.

Isotopic enrichment at certain positions of a compound provided herein may produce a detectable KIE that affects the pharmacokinetic, pharmacologic, and/or toxicological profiles of a compound provided herein in comparison with a similar compound having a natural isotopic composition. In one embodiment, the deuterium enrichment is performed on the site of C—H bond cleavage during metabolism.

Certain embodiments herein provide deuterium enriched 5-azacytidine analogs, wherein one or more hydrogen(s) in the 5-azacytidine molecule is/are isotopically enriched with deuterium. In certain embodiments, provided herein are compounds of formula (I):

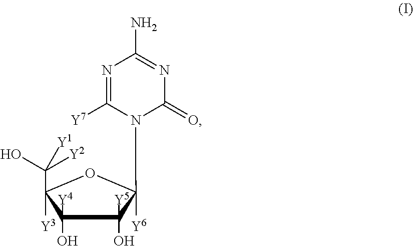

wherein one or more Y atom(s) (i.e., Y¹, Y², Y³, Y⁴, Y⁵, Y⁶, and Y⁷) is/are hydrogen(s) isotopically enriched with deuterium, and any remaining Y atom(s) is/are non-enriched hydrogen atom(s). In particular embodiments, one, two, three, four, five, six, or seven of the indicated Y atom(s) is/are isotopically enriched with deuterium, and any remaining Y atom(s) is/are non-enriched hydrogen(s).

In certain embodiments, one or more Y atoms on the ribose moiety of Compound (I) are deuterium-enriched. Particular examples include, but are not limited to, the following compounds, in which the label “D” indicates a deuterium-enriched atomic position, i.e., a sample comprising the given compound has a deuterium enrichment at the indicated position(s) above the natural abundance of deuterium:

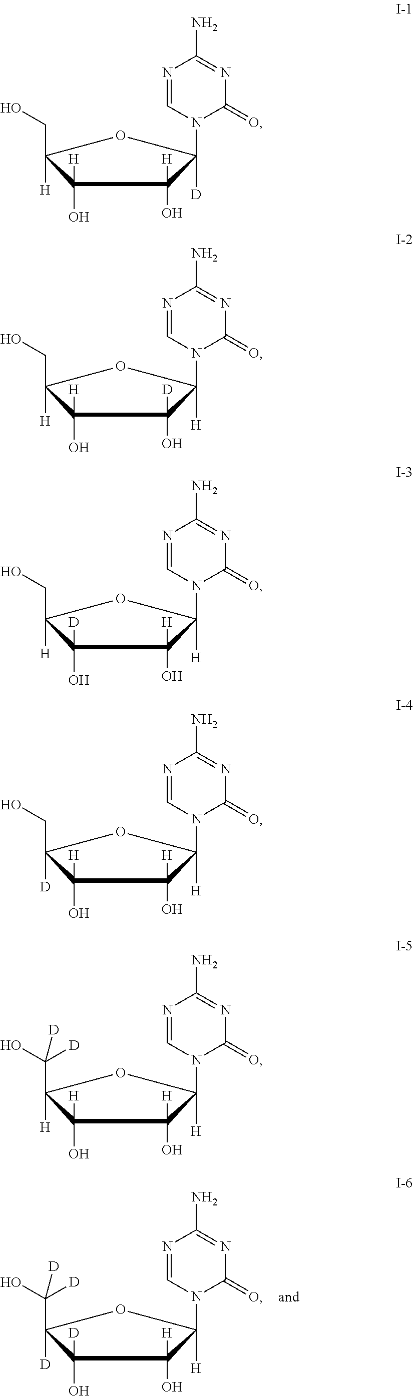

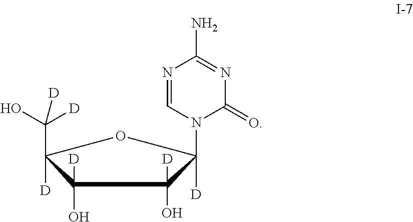

In certain embodiments, the Y atom on the 5-azacytosine moiety of Compound (I) is deuterium-enriched. Particular example includes the following compound, in which the label “D” indicates a deuterium-enriched atomic position, i.e., a sample comprising the given compound has a deuterium enrichment at the indicated position(s) above the natural abundance of deuterium:

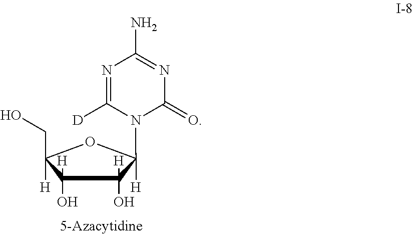

In certain embodiments, one or more Y atoms on the ribose moiety and the Y atom on the 5-azacytosine moiety of Compound (I) are deuterium-enriched. Particular examples include, but are not limited to, the following compounds, in which the label “D” indicates a deuterium-enriched atomic position, i.e., a sample comprising the given compound has a deuterium enrichment at the indicated position(s) above the natural abundance of deuterium:

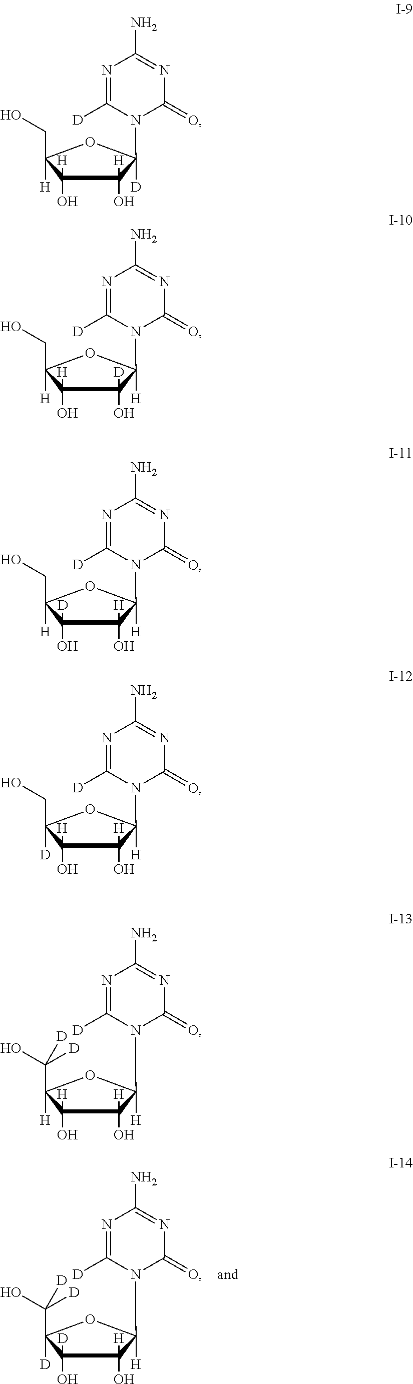

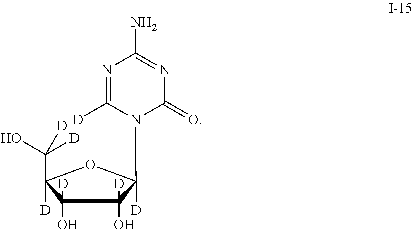

It is understood that one or more deuterium(s) may exchange with hydrogen under physiological conditions.

Certain embodiments herein provide carbon-13 enriched analogs of 5-azacytidine, wherein one or more carbon(s) in the 5-azacytidine molecule is/are isotopically enriched with carbon-13. In certain embodiments, provided herein are compounds of formula (II):

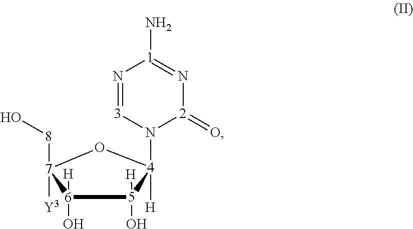

wherein one or more of 1, 2, 3, 4, 5, 6, 7, or 8 is/are carbon atom(s) isotopically enriched with carbon-13, and any remaining atom(s) of 1, 2, 3, 4, 5, 6, 7, or 8 is/are non-enriched carbon atom(s). In particular embodiments, one, two, three, four, five, six, seven, or eight carbon atom(s) (i.e., atoms 1, 2, 3, 4, 5, 6, 7, and 8) is/are isotopically enriched with carbon-13, and any remaining carbon atom(s) is/are non-enriched.

In certain embodiments, one or more carbon atom(s) of the ribose moiety of Compound (II) are enriched with carbon-13. Particular examples include, but are not limited to, the following compounds, in which the asterisk (“*”) indicates a carbon-13 enriched atomic position, i.e., a sample comprising the given compound has a carbon-13 enrichment at the indicated position(s) above the natural abundance of carbon-13:

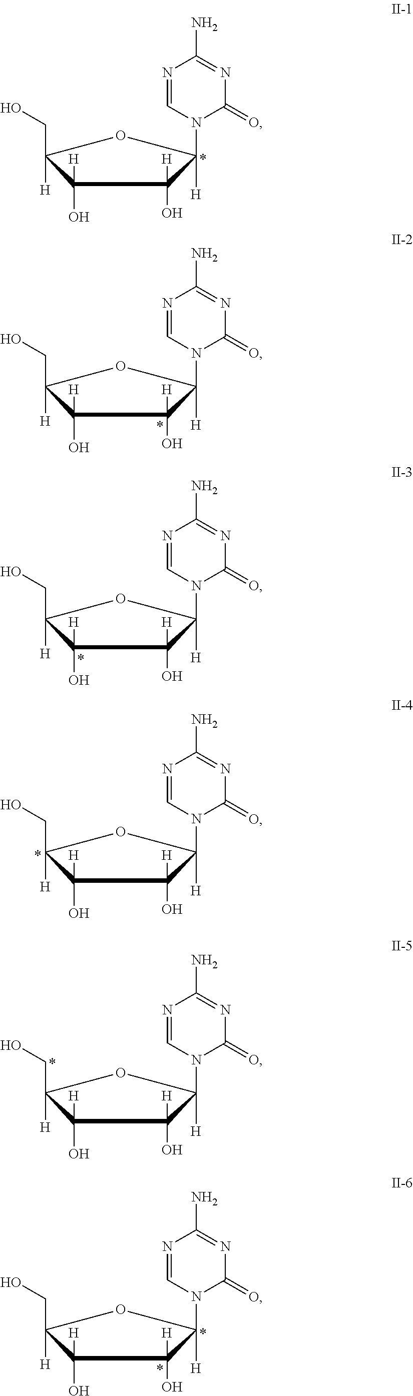

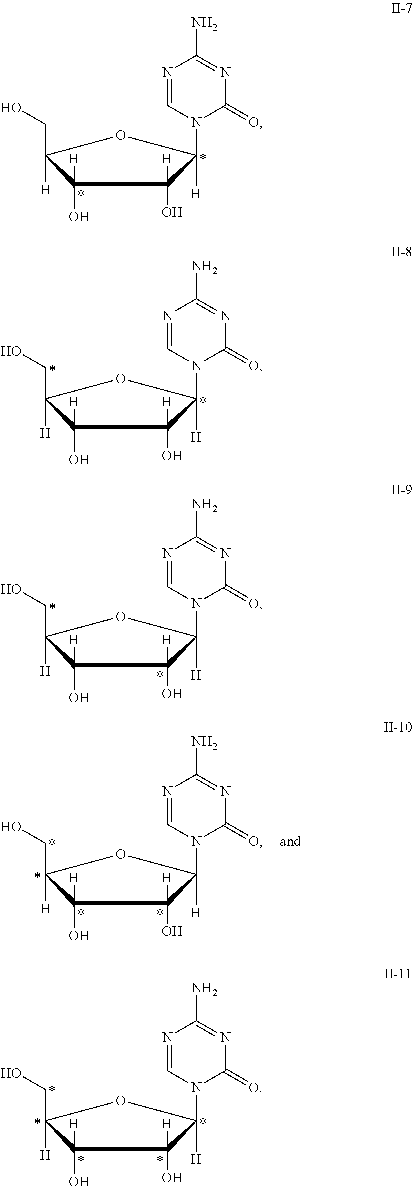

In certain embodiments, one or more carbon atom(s) of the 5-azacytosine moiety of Compound (II) are enriched with carbon-13. Particular examples include, but are not limited to, the following compounds, in which the asterisk “*” indicates a carbon-13 enriched atomic position, i.e., a sample comprising the given compound has a carbon-13 enrichment at the indicated position(s) above the natural abundance of carbon-13:

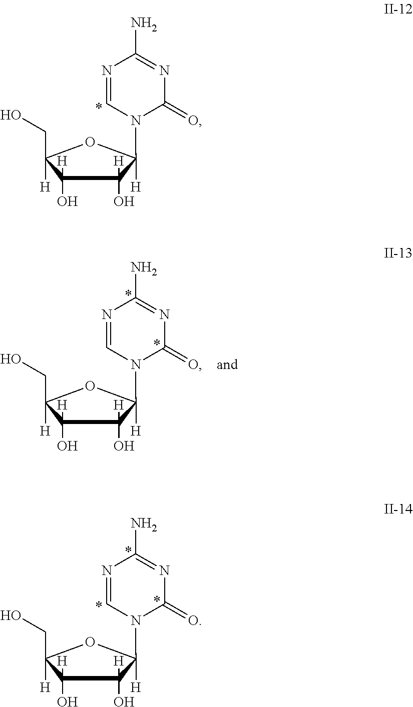

In certain embodiments, one or more carbon atoms on the ribose moiety and one or more carbon atoms on the 5-azacytosine moiety of Compound (11) are enriched with carbon-13, i.e., any combination of carbon-13 enrichment for the ribose moiety and carbon-13 enrichment for the azacitosine moiety is encompassed herein.

In certain embodiments, one or more hydrogen(s) is/are enriched with deuterium(s) and one or more carbon(s) is/are enriched with carbon-13, i.e., any combination of deuterium enrichment and carbon-13 enrichment of 5-azacytidine is encompassed herein.

3. Synthesis of Isotopically Enriched Cytidine Analogs

The compounds described herein may be synthesized using any method known to one of ordinary skill in the art. For example, particular compounds described herein are synthesized using standard synthetic organic chemistry techniques known to those of ordinary skill in the art. In some embodiments, known procedures for the synthesis of 5-azacytidine are employed, wherein one or more of the reagents, starting materials, precursors, or intermediates are replaced by one or more isotopically-enriched reagents, starting materials, precursors, or intermediates, including but not limited to one or more deuterium-enriched reagents, starting materials, precursors, or intermediates, and/or one or more carbon-13-enriched reagents, starting materials, precursors, or intermediates. Isotopically enriched reagents, starting materials, precursors, or intermediates are commercially available or may be prepared by routine chemical reactions known to one of skill in the art. In some embodiments, the routes are based on those disclosed in U.S. Pat. No. 7,038,038, which is incorporated herein by reference in its entirety.

In certain embodiments, a suitable isotopically enriched starting material, such as a deuterium-enriched ribose, a deuterium-enriched 5-azacytosine, a carbon-13-enriched ribose, and/or a carbon-13-enriched 5-azacytosine, may be employed as the starting material in the following general scheme to prepare the corresponding deuterium and/or carbon-13 enriched 5-azacytidine (See Scheme 1). Following the procedures in U.S. Pat. No. 7,038,038, 5-azacytosine is treated with hexamethyldisilazane (HMDS) to render a silylated 5-azacytosine. Tetraacetyl-D-ribose is prepared by reacting D-ribose with sodium acetate in acetic anhydride, following the procedures in Brown et al., Biochemical Preparations, 1955, 4, 70-76. The silylated 5-azacytosine is coupled to tetraacetyl-D-ribose in the presence of TMS-triflate, and the resulting protected 5-azacytidine is treated with sodium methoxide in methanol to yield 5-azacytidine. See U.S. Pat. No. 7,038,038.

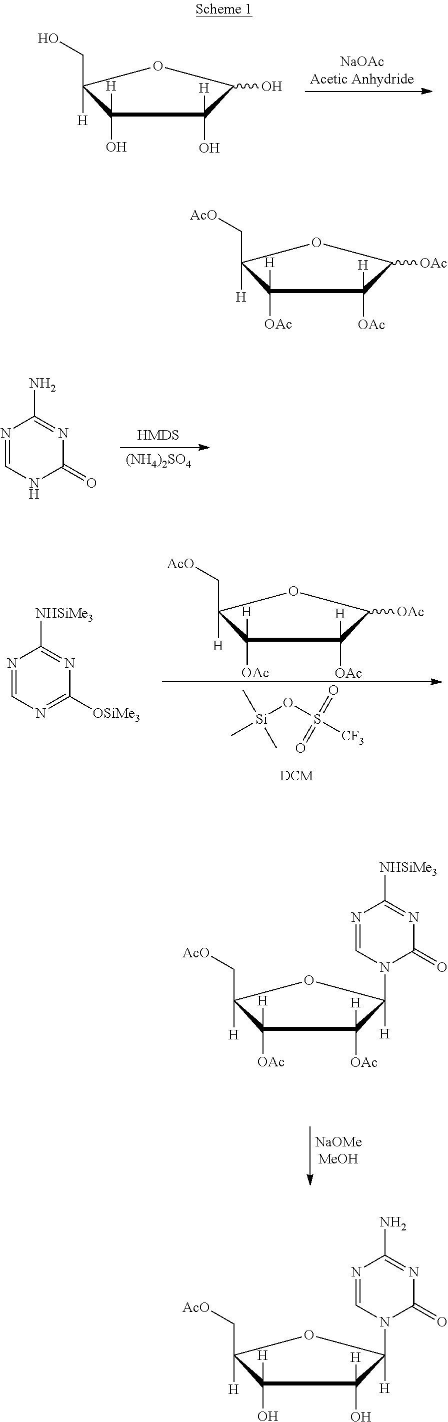

In some embodiments, one or more hydrogen positions in the ribose portion of 5-azacytidine are enriched with deuterium. Such 5-azacytidine analogs may be prepared following Scheme 1 from a suitable deuterium-enriched ribose, purchased from a commercial source or prepared following literature procedures. Specific examples of deuterium-enriched ribose starting material include, but are not limited to, the following compounds listed in Table 1, which may be converted to the corresponding deuterium-enriched 5-azacytidine analogs.

TABLE 1

| | | | 5-Aza- |
|---|---|---|---|
| Starting | | | cylidine |
| Material | Structure | Source/Reference | Product |
| D-Ribose- 1-D | 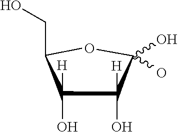 | Cambridge Isotope Lab. | I-1 |
| D-Ribose- 2-D | 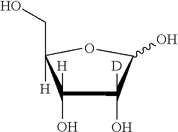 | Cambridge Isotope Lab. | I-2 |
| D-Ribose- 3-D | 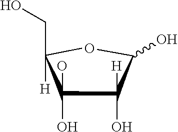 | Omicron Biochemicals, Inc, | I-3 |
| D-Ribose- 4-D | 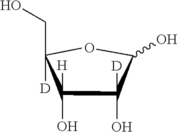 | Omicron Biochemicals, Inc. | I-4 |
| D-Ribose- 5,5′- D₂ | 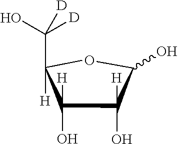 | Omicron Biochemicals, Inc. | I-5 |
| D-Ribose- 3,4,5, 5′-D₄ | 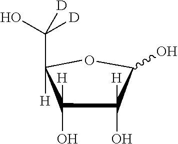 | Prepared following the procedures in J. Am, Chem, Soc. 1996, 118, 7929-7940. | I-6 |

In other embodiments, the hydrogen position on the 5-azacytosine ring of 5-azacytidine is enriched with deuterium. Such 5-azacytidine analog may be prepared, e.g., from deuterated 5-azacytosine following Scheme 1. The deuterated 5-azacytosine may be prepared, e.g., from suitable deuterated reagents as shown in Scheme 2. See e.g., Grundmann et al., Chem. Ber. 1954, 87, 19-24; Piskala et al., in Zorbach and Tipson (eds.) Synthetic Procedures in Nucleic Acid Chemistry, Vol. 1, Wiley Interscience, New York, 1968, 107-108; Piskala, Collect. Czech. Chem. Comm. 1967, 32, 3966-3976.

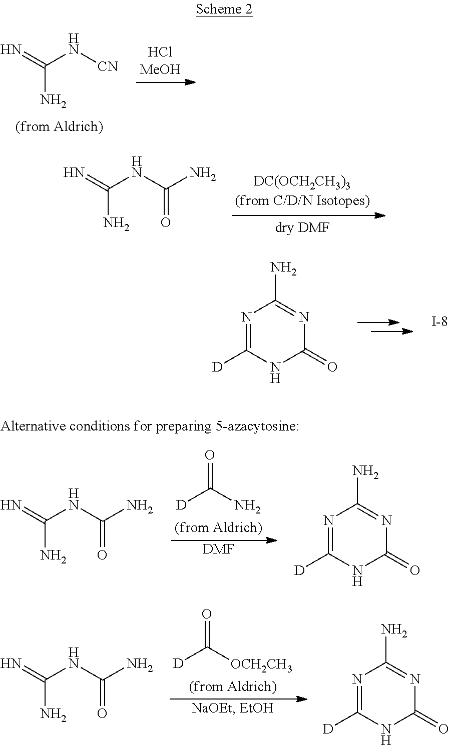

In other embodiments, both the hydrogen position on the 5-azacytosine ring and one or more hydrogen positions in the ribose portion of 5-azacytidine are enriched with deuterium. Such 5-azacytidine analogs may be prepared, e.g., following Scheme 1, coupling a suitable deuterated ribose starting materials with deuterated 5-azacytosine. For example, compounds 1-9, 1-10, 1-11, 1-12, 1-13, and 1-14 may be prepared from the corresponding deuterated ribose starting material listed in Table 1, and deuterated 5-azacytosine prepared according to Scheme 2.

In some embodiments, one or more carbon atoms in the ribose portion of 5-azacytidine are enriched with carbon-13. Such 5-azacytidine analogs may be prepared following Scheme 1 from a suitable carbon-13-enriched ribose, purchased from a commercial source or prepared following literature procedures. Specific examples of carbon-13-enriched ribose starting material include, but are not limited to, the following compounds listed in Table 2, which may be converted to the corresponding carbon-13-enriched 5-azacytidine analogs. (The asterisk “*” indicates a carbon-13 enriched atomic position)

TABLE 2

| | | | 5-Aza- |
|---|---|---|---|
| Starting | | Source/ | cytidine |
| Material | Structure | Reference | Product |
| D -Ribose- 1-¹³C | 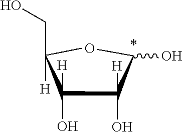 | Sigma Aldrich | II-1 |
| D-Ribose- 2-¹³C | 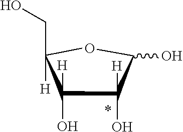 | Sigma Aldrich | II-2 |
| D-Ribose- 3-¹³C | 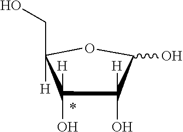 | Omicron Biochemicals, Inc. | II-3 |
| D-Ribose- 4-¹³C | 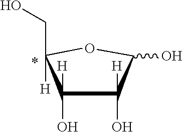 | Omicron Biochemicals, Inc. | II-4 |
| D-Ribose- 5-¹³C | 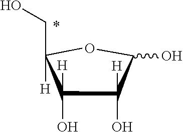 | Cambridge Isotope Lab. | II-5 |
| D-Ribose- 1,2-¹³C₂ | 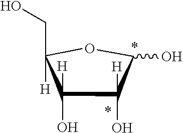 | Sigma Aldrich | II-6 |
| D-Ribose- 1,3-¹³C₂ | 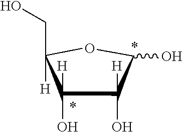 | Omicron Biochemicals, Inc. | II-7 |
| D-Ribose- 1,5-¹³C₂ | 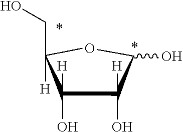 | Omicron Biochemicals, Inc. | II-8 |
| D-Ribose- 2,5-¹³C₂ | 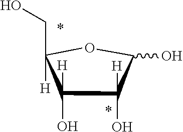 | Omicron Biochemicals, Inc. | II-9 |
| D-Ribose- 2,3,4,5-¹³C₄ | 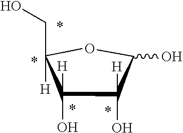 | Sigma Aldrich | II-10 |
| D-Ribose- 1,2,3,4,5-¹³C₅ | 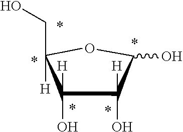 | Cambridge Isotope Lab. | II-11 |

In other embodiments, one or more carbon atoms in the 5-azacytosine ring are enriched with carbon-13. Such 5-azacytidine analogs may be prepared from a carbon-13-enriched 5-azacytosine following Scheme 1. The carbon-13 enriched 5-azacytosine intermediates may be prepared from suitable carbon-13 enriched reagents as shown in Scheme 3. See e.g., Grundmann et al., Chem. Ber. 1954, 87, 19-24; Piskala et al., in Zorbach and Tipson (eds.) Synthetic Procedures in Nucleic Acid Chemistry, Vol. 1, Wiley Interscience, New York, 1968, 107-108; Piskala, Collect. Czech. Chem. Comm. 1967, 32, 3966-3976.

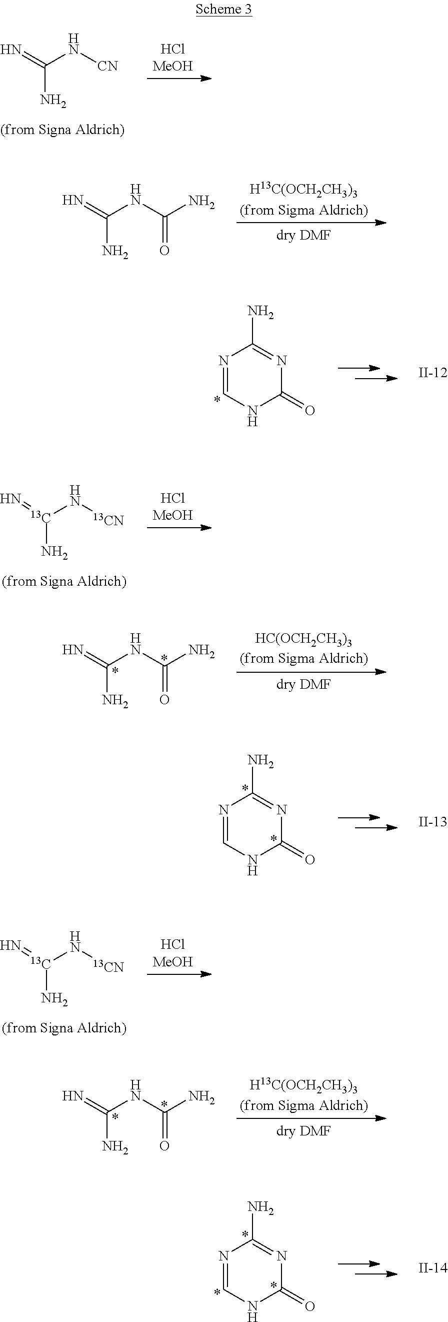

In other embodiments, one or more carbon positions on the 5-azacytosine ring and one or more carbon positions in the ribose portion of 5-azacytidine are enriched with carbon-13. Such 5-azacytidine analogs may be prepared following Scheme 1, coupling a suitable carbon-13-enriched ribose starting materials with a suitable carbon-13-enriched 5-azacytosine. For example, compounds may be prepared from a carbon-13-enriched ribose starting material listed in Table 2, and carbon-13-enriched 5-azacytosine prepared according to Scheme 3.

The routes and methods described above may be modified to provide an isotopolougue of 5-azacytidine having both deuterium enrichment and carbon-13 enrichment.

### C. Pharmaceutical Formulations

1. Overview

Embodiments herein encompass pharmaceutical formulations and compositions comprising one or more cytidine analogs, e.g., 5-azacytidine, and optionally a permeation enhancer, wherein the formulations and compositions are prepared for oral administration. In a particular embodiment, the formulations and compositions are prepared for release of the cytidine analog substantially in the stomach. In specific embodiments, the cytidine analogs, e.g., 5-azacytidine, and the pharmaceutical formulations and compositions are used for treating diseases and disorders associated with abnormal cell proliferation, wherein the cytidine analogs, the formulations and compositions are prepared for oral administration, preferably for release of the cytidine analogs substantially in the stomach. Particular embodiments relate to the use of one or more cytidine analogs, e.g., 5-azacytidine, for the preparation of pharmaceutical formulations and compositions for treating particular medical indications, as provided herein. The pharmaceutical formulations and compositions comprising cytidine analogs provided herein are intended for oral delivery of the cytidine analog in subjects in need thereof. Oral delivery formats include, but are not limited to, tablets, capsules, caplets, solutions, suspensions, and syrups, and may also comprise a plurality of granules, beads, powders or pellets that may or may not be encapsulated. Such formats may also be referred to herein as the “drug core” which contains the cytidine analog.

Particular embodiments herein provide solid oral dosage forms that are tablets or capsules. In certain embodiments, the formulation is a tablet comprising a cytidine analog. In certain embodiments, the formulation is a capsule comprising a cytidine analog. In certain embodiments, the tablets or capsules provided herein optionally comprise one or more excipients, such as, for example, glidants, diluents, lubricants, colorants, disintegrants, granulating agents, binding agents, polymers, and coating agents. In certain embodiments, the formulation is an immediate release tablet. In certain embodiments, the formulation is a controlled release tablet releasing the API, e.g., substantially in the stomach. In certain embodiments, the formulation is a hard gelatin capsule. In certain embodiments, the formulation is a soft gelatin capsule. In certain embodiments, the capsule is a hydroxypropyl methylcellulose (HPMC) capsule. In certain embodiments, the formulation is an immediate release capsule. In certain embodiments, the formulation is an immediate or controlled release capsule releasing the API, e.g., substantially in the stomach. In certain embodiments, the formulation is a rapidly disintegrating tablet that dissolves substantially in the mouth following administration. In certain embodiments, embodiments herein encompass the use of cytidine analogs, e.g., 5-azacytidine, for the preparation of a pharmaceutical composition for treating a disease associated with abnormal cell proliferation, wherein the composition is prepared for oral administration.

2. Performance of Certain Dosage Forms Provided Herein

In certain embodiments, the formulations comprising the cytidine analogs, such as, for example, 5-azacytidine, effect an immediate release of the API upon oral administration. In particular embodiments, the formulations comprising the cytidine analogs, such as, for example, 5-azacytidine, comprise a therapeutically or prophylactically effective amount of the cytidine analog (and, optionally, one or more excipients) and effect an immediate release of the API upon oral administration.

In certain embodiments, the formulations comprising the cytidine analogs, such as, for example, 5-azacytidine, effect a controlled release of the API substantially in the stomach upon oral administration. In certain embodiments, the formulations comprising the cytidine analogs, such as, for example, 5-azacytidine, comprise a therapeutically or prophylactically effective amount of the cytidine analog and a drug release controlling component which is capable of releasing the cytidine analog substantially in the stomach. In certain embodiments, matrices (e.g., polymer matrices) may be employed in the formulation to control the release of the cytidine analog. In certain embodiments, coatings and/or shells may be employed in the formulation to control the release of the cytidine analog in the substantially in the stomach.

In certain embodiments, the formulations comprising the cytidine analogs, such as, for example, 5-azacytidine, release the API substantially in the stomach upon oral administration. In certain embodiments, the formulations effect an immediate release of the cytidine analog upon oral administration. In certain embodiments, the formulations optionally further comprises a drug release controlling component, wherein the drug release controlling component is adjusted such that the release of the cytidine analog occurs substantially in the stomach. In particular embodiments, the drug release controlling component is adjusted such that the release of the cytidine analog is immediate and occurs substantially in the stomach. In particular embodiments, the drug release controlling component is adjusted such that the release of the cytidine analog is sustained and occurs substantially in the stomach. In certain embodiments, the formulation of the cytidine analog, such as, for example, 5-azacytidine, releases the API substantially in the stomach, and, subsequently, releases the remainder of the API in the intestine upon oral administration.

Methods by which skilled practitioners can assess where a drug is released in the gastrointestinal tract of a subject are known in the art, and include, for example, scintigraphic studies, testing in a bio-relevant medium which simulates the fluid in relevant portions of the gastrointestinal tract, among other methods.

Particular embodiments herein provide pharmaceutical formulations (e.g., immediate release oral formulations and/or formulations that release the API substantially in the stomach) comprising a cytidine analog (e.g., 5-azacytidine) that achieve a particular exposure in the subject to which the formulation is orally administered, as compared to a SC dose of the same cytidine analog. Particular embodiments provide oral formulations that achieve an exposure of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%, as compared to a SC dose.

In certain embodiments, the formulation (e.g., immediate release oral formulation and/or formulation that release the API substantially in the stomach) comprising the cytidine analog, such as, for example, 5-azacytidine, renders a certain percentage of the cytidine analog in the formulation systemically bioavailable upon oral administration. In certain embodiments, after the subject is orally administered the formulation, the cytidine analog in the formulation is absorbed substantially in the stomach, and becomes available to the body through systemic exposure. In particular embodiments, the oral bioavailability of a formulation comprising a cytidine analog provided herein is, e.g., greater than about 1%, greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, or about 100%, of the total amount of the cytidine analog in the formulation.

Methods by which skilled practitioners can assess the oral bioavailability of a drug formulation in a subject are known in the art. Such methods, include, for example, comparing certain dosing-related parameters, such as, but not limited to, maximum plasma concentration (“Cmax”), time to maximum plasma concentration (“Tmax”), or area-under-the-curve (“AUC”) determinations.

Particular embodiments herein provide pharmaceutical formulations (e.g., immediate release oral formulations and/or formulations that release the API substantially in the stomach) comprising a cytidine analog (e.g., 5-azacytidine) that achieve a particular AUC value (e.g., AUC(0-t) or AUC(0-∞)) in the subject (e.g., human) to which the formulation is orally administered. Particular embodiments provide oral formulations that achieve an AUC value of at least about 25 ng-hr/mL, at least about 50 ng-hr/mL, at least about 75 ng-hr/mL, at least about 100 ng-hr/mL, at least about 150 ng-hr/mL, at least about 200 ng-hr/mL, at least about 250 ng-hr/mL, at least about 300 ng-hr/mL, at least about 350 ng-hr/mL, at least about 400 ng-hr/mL, at least about 450 ng-hr/mL, at least about 500 ng-hr/mL, at least about 550 ng-hr/mL, at least about 600 ng-hr/mL, at least about 650 ng-hr/mL, at least about 700 ng-hr/mL, at least about 750 ng-hr/mL, at least about 800 ng-hr/mL, at least about 850 ng-hr/mL, at least about 900 ng-hr/mL, at least about 950 ng-hr/mL, at least about 1000 ng-hr/mL, at least about 1100 ng-hr/mL, at least about 1200 ng-hr/mL, at least about 1300 ng-hr/mL, at least about 1400 ng-hr/mL, at least about 1500 ng-hr/mL, at least about 1600 ng-hr/mL, at least about 1700 ng-hr/mL, at least about 1800 ng-hr/mL, at least about 1900 ng-hr/mL, at least about 2000 ng-hr/mL, at least about 2250 ng-hr/mL, or at least about 2500 ng-hr/mL. In particular embodiments, the AUC determination is obtained from a time-concentration pharmacokinetic profile obtained from the blood samples of animals or human volunteers following dosing.

Particular embodiments herein provide pharmaceutical formulations (e.g., immediate release oral formulations and/or formulations that release the API substantially in the stomach) comprising a cytidine analog (e.g., 5-azacytidine) that achieve a particular maximum plasma concentration (“Cmax”) in the subject to which the formulation is orally administered. Particular embodiments provide oral formulations that achieve a Cmax of the cytidine analog of at least about 25 ng/mL, at least about 50 ng/mL, at least about 75 ng/mL, at least about 100 ng/mL, at least about 150 ng/mL, at least about 200 ng/mL, at least about 250 ng/mL, at least about 300 ng/mL, at least about 350 ng/mL, at least about 400 ng/mL, at least about 450 ng/mL, at least about 500 ng/mL, at least about 550 ng/mL, at least about 600 ng/mL, at least about 650 ng/mL, at least about 700 ng/mL, at least about 750 ng/mL, at least about 800 ng/mL, at least about 850 ng/mL, at least about 900 ng/mL, at least about 950 ng/mL, at least about 1000 ng/mL, at least about 1100 ng/mL, at least about 1200 ng/mL, at least about 1300 ng/mL, at least about 1400 ng/mL, at least about 1500 ng/mL, at least about 1600 ng/mL, at least about 1700 ng/mL, at least about 1800 ng/mL, at least about 1900 ng/mL, at least about 2000 ng/mL, at least about 2250 ng/mL, or at least about 2500 ng/mL.

Particular embodiments herein provide pharmaceutical formulations (e.g., immediate release oral formulations and/or formulations that release the API substantially in the stomach) comprising a cytidine analog (e.g., 5-azacytidine) that achieve a particular time to maximum plasma concentration (“Tₘₐₓ”) in the subject to which the formulation is orally administered. Particular embodiments provide oral formulations that achieve a Tₘₐₓ of the cytidine analog of less than about 10 min., less than about 15 min., less than about 20 min., less than about 25 min., less than about 30 min., less than about 35 min., less than about 40 min., less than about 45 min., less than about 50 min., less than about 55 min., less than about 60 min., less than about 65 min., less than about 70 min., less than about 75 min., less than about 80 min., less than about 85 min., less than about 90 min., less than about 95 min., less than about 100 min., less than about 105 min., less than about 110 min., less than about 115 min., less than about 120 min., less than about 130 min., less than about 140 min., less than about 150 min., less than about 160 min., less than about 170 min., less than about 180 min., less than about 190 min., less than about 200 min., less than about 210 min., less than about 220 min., less than about 230 min., or less than about 240 min. In particular embodiments, the Tₘₐₓ value is measured from the time at which the formulation is orally administered.

Particular embodiments herein provide oral dosage forms comprising a cytidine analog, wherein the oral dosage forms have an enteric coating. Particular embodiments provide a permeable or partly permeable (e.g., “leaky”) enteric coating with pores. In particular embodiments, the permeable or partly permeable enteric-coated tablet releases the 5-azacytidine in an immediate release manner substantially in the stomach.

3. Design of Certain Dosage Forms Provided Herein

Provided herein are dosage forms designed to maximize the absorption and/or efficacious delivery of certain cytidine analogs, e.g., 5-azacytidine, upon oral administration, e.g., for release substantially in the stomach. Accordingly, certain embodiments herein provide a solid oral dosage form of a cytidine analog, such as, for example, 5-azacytidine, using pharmaceutical excipients designed for immediate release of the API upon oral administration, e.g., substantially in the stomach. Particular immediate release formulations comprise a specific amount of a cytidine analog and optionally one or more excipients. In certain embodiments, the formulation may be an immediate release tablet or an immediate release capsule (such as, e.g., an HPMC capsule).

Provided herein are methods of making the formulations provided herein comprising the cytidine analogs provided herein (e.g., immediate release oral formulations and/or formulations that release the API substantially in the stomach). In particular embodiments, the formulations provided herein may be prepared using conventional methods known to those skilled in the field of pharmaceutical formulation, as described, e.g., in pertinent textbooks. See, e.g., REMINGTON, THE SCIENCE AND PRACTICE OF PHARMACY, 20th Edition, Lippincott Williams & Wilkins, (2000); ANSEL et al., PHARMACEUTICAL DOSAGE FORMS AND DRUG DELIVERY SYSTEMS, 7th Edition, Lippincott Williams & Wilkins, (1999); GIBSON, PHARMACEUTICAL PREFORMULATION AND FORMULATION, CRC Press (2001).

In particular embodiments, formulations provided herein (e.g., immediate release oral formulations, formulations that release the API substantially in the stomach, or rapidly disintegrating formulations that dissolve substantially in the mouth) comprise a cytidine analog, such as, for example, 5-azacytidine, in a specific amount. In particular embodiments, the specific amount of the cytidine analog in the formulation is, e.g., about 10 mg, about 20 mg, about 40 mg, about 60 mg, about 80 mg, about 100 mg, about 120 mg, about 140 mg, about 160 mg, about 180 mg, about 200 mg, about 220 mg, least about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 3000 mg, about 4000 mg, or about 5000 mg. In particular embodiments, the specific amount of the cytidine analog in the formulation is, e.g., at least about 10 mg, at least about 20 mg, at least about 40 mg, at least about 60 mg, at least about 80 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 600 mg, at least about 700 mg, at least about 800 mg, at least about 900 mg, at least about 1000 mg, at least about 1100 mg, at least about 1200 mg, at least about 1300 mg, at least about 1400 mg, at least about 1500 mg, at least about 1600 mg, at least about 1700 mg, at least about 1800 mg, at least about 1900 mg, at least about 2000 mg, at least about 2100 mg, at least about 2200 mg, at least about 2300 mg, at least about 2400 mg, at least about 2500 mg, at least about 3000 mg, at least about 4000 mg, or at least about 5000 mg.

In certain embodiments, the formulation is a tablet, wherein the tablet is manufactured using standard, art-recognized tablet processing procedures and equipment. In certain embodiments, the method for forming the tablets is direct compression of a powdered, crystalline and/or granular composition comprising the cytidine analog, alone or in combination with one or more excipients, such as, for example, carriers, additives, polymers, or the like. In certain embodiments, as an alternative to direct compression, the tablets may be prepared using wet granulation or dry granulation processes. In certain embodiments, the tablets are molded rather than compressed, starting with a moist or otherwise tractable material. In certain embodiments, compression and granulation techniques are used.

In certain embodiments, the formulation is a capsule, wherein the capsules may be manufactured using standard, art-recognized capsule processing procedures and equipments. In certain embodiments, soft gelatin capsules may be prepared in which the capsules contain a mixture of the cytidine analog and vegetable oil or non-aqueous, water miscible materials such as, for example, polyethylene glycol and the like. In certain embodiments, hard gelatin capsules may be prepared containing granules of the cytidine analog in combination with a solid pulverulent carrier, such as, for example, lactose, saccharose, sorbitol, mannitol, potato starch, corn starch, amylopectin, cellulose derivatives, or gelatin. In certain embodiments, a hard gelatin capsule shell may be prepared from a capsule composition comprising gelatin and a small amount of plasticizer such as glycerol. In certain embodiments, as an alternative to gelatin, the capsule shell may be made of a carbohydrate material. In certain embodiments, the capsule composition may additionally include polymers, colorings, flavorings and opacifiers as required. In certain embodiments, the capsule comprises HPMC.

In certain embodiments, the formulation of the cytidine analog, such as, for example, 5-azacytidine, is prepared using aqueous solvents without causing significant hydrolytic degradation of the cytidine analog. In particular embodiments, the formulation of the cytidine analog, such as, for example, 5-azacytidine, is a tablet which contains a coating applied to the drug core using aqueous solvents without causing significant hydrolytic degradation of the cytidine analog in the formulation. In certain embodiments, water is employed as the solvent for coating the drug core. In certain embodiments, the oral dosage form of the cytidine analog is a tablet containing a film coat applied to the drug core using aqueous solvents. In particular embodiments, water is employed as the solvent for film-coating. In particular embodiments, the tablet containing the cytidine analog is film-coated using aqueous solvents without effecting degradation of the pharmaceutical composition. In particular embodiments, water is used as the film coating solvent without effecting degradation of the pharmaceutical composition. In certain embodiments, an oral dosage form comprising 5-azacytidine and an aqueous film coating effects immediate drug release upon oral delivery. In certain embodiments, the oral dosage form comprising 5-azacytidine and an aqueous film coating effects controlled drug release to the upper gastrointestinal tract, e.g., the stomach, upon oral administration. In particular embodiments, a tablet with an aqueous-based film coating comprises 5-azacytidine as the API.

In certain embodiments, provided herein is a controlled release pharmaceutical formulation for oral administration of a cytidine analog that releases the cytidine analog substantially in the stomach, comprising: a) a specific amount of a cytidine analog; b) a drug release controlling component for controlling the release of the cytidine analog substantially in the upper gastrointestinal tract, e.g., the stomach; and c) optionally one or more excipients. In certain embodiments, the oral dosage form comprising the cytidine analog is prepared as a controlled release tablet or capsule which includes a drug core comprising the pharmaceutical composition and optional excipients. Optionally, a “seal coat” or “shell” is applied. In certain embodiments, a formulation provided herein comprising a cytidine analog provided herein is a controlled release tablet or capsule, which comprises a therapeutically effective amount of the cytidine analog, a drug release controlling component that controls the release of the cytidine analog substantially in the stomach upon oral administration, and optionally, one or more excipients.

Particular embodiments provide a drug release controlling component that is a polymer matrix, which swells upon exposure to gastric fluid to effect the gastric retention of the formulation and the sustained release of the cytidine analog from the polymer matrix substantially in the stomach. In certain embodiments, such formulations may be prepared by incorporating the cytidine analog into a suitable polymeric matrix during formulation. Examples of such formulations are known in the art. See, e.g., Shell et al., U.S. Patent Publication No. 2002/0051820 (application Ser. No. 09/990,061); Shell et al., U.S. Patent Publication No. 2003/0039688 (Application Ser. No. 10/045,823); Gusler et al., U.S. Patent Publication No. 2003/0104053 (Application Ser. No. 10/029,134), each of which is incorporated herein by reference in its entirety.

In certain embodiments, the drug release controlling component may comprise a shell surrounding the drug-containing core, wherein the shell releases the cytidine analog from the core by, e.g., permitting diffusion of the cytidine analog from the core and promoting gastric retention of the formulation by swelling upon exposure to gastric fluids to a size that is retained in the stomach. In certain embodiments, such formulations may be prepared by first compressing a mixture of the cytidine analog and one or more excipients to form a drug core, and compressing another powdered mixture over the drug core to form the shell, or enclosing the drug core with a capsule shell made of suitable materials. Examples of such formulations are known in the art. See, e.g., Berner et al., U.S. Patent Publication No. 2003/0104062 application Ser. No. 10/213,823), incorporated herein by reference in its entirety.

Certain embodiments herein provide oral dosage forms comprising a cytidine analog, wherein the dosage form contains pores in the conventional enteric coating. In particular embodiments, the oral dosage form of the cytidine analog is a tablet that contains a permeable or partly permeable (e.g., “leaky”) enteric coating with pores. In particular embodiments, the permeable or partly permeable enteric-coated tablet controls the release of the cytidine analog from the tablet primarily to the upper gastrointestinal tract, e.g., the stomach. In particular embodiments, the permeable or partly permeable enteric-coated tablet comprises 5-azacytidine. In particular embodiments, the remainder of the cytidine analog is subsequently released beyond the stomach (e.g., in the intestine).

In certain embodiments, the pharmaceutical formulation provided herein is a compressed tablet comprising a cytidine analog. In addition to the cytidine analog, the tablet optionally comprises one or more excipients, including (a) diluents or fillers, which may add necessary bulk to a formulation to prepare tablets of the desired size; (b) binders or adhesives, which may promote adhesion of the particles of the formulation, enabling a granulation to be prepared and maintaining the integrity of the final tablet; (c) disintegrants or disintegrating agents, which, after administration, may promote breakup of the tablets to smaller particles for improved drug availability; (d) anti-adherents, glidants, lubricants or lubricating agents, which may enhance flow of the tableting material into the tablet dies, minimize wear of the punches and dies, prevent the sticking of fill material to the punches and dies, and produce tablets having a sheen; and (e) miscellaneous adjuncts such as colorants and flavorants. After compression, tablets provided herein may be coated with various materials as described herein.

In certain embodiments, the pharmaceutical formulation provided herein is a multiple compressed tablet of a cytidine analog. Multiple compressed tablets are prepared by subjecting the fill material to more than a single compression. The result may be a multiple-layered tablet or a tablet-within-a-tablet, the inner tablet being the core comprising a cytidine analog and optionally one or more excipients, and the outer portion being the shell, wherein the shell comprises one or more excipients, and may or may not contain the cytidine analog. Layered tablets may be prepared by the initial compaction of a portion of fill material in a die followed by additional fill material and compression to form two- or three-layered tablets, depending upon the number of separate fills. Each layer may contain a different therapeutic agent, separate from one another for reasons of chemical or physical incompatibility, or the same therapeutic agent for staged drug release, or simply for the unique appearance of the multiple-layered tablet. Each portion of fill may be colored differently to prepare a distinctive looking tablet. In the preparation of tablets having a compressed tablet as the inner core, special machines may be used to place the preformed tablet precisely within the die for the subsequent compression of surrounding fill material.

In certain embodiments, the compressed tablet of a cytidine analog may be coated with a colored or an uncolored sugar layer. The coating may be water-soluble and quickly dissolved after oral ingestion. The sugar coating may serve the purpose of protecting the enclosed drug from the environment and providing a barrier to an objectionable taste or smell. The sugar coating may also enhance the appearance of the compressed tablet and permit the imprinting of identifying manufacturer's information. In certain embodiments, sugar-coated tablets may be 50% larger and heavier than the original uncoated tablets. The sugar-coating of tablets may be divided into the following optional steps: (1) waterproofing and sealing (if needed); (2) sub-coating; (3) smoothing and final rounding; (4) finishing and coloring (if desired); (5) imprinting (if needed); and (6) polishing.

In certain embodiments, the compressed tablet of a cytidine analog may be film-coated. Film-coated tablets may be compressed tablets coated with a thin layer of a polymer capable of forming a skin-like film over the tablet. The film is usually colored and has the advantage to be more durable, less bulky, and less time-consuming to apply. By its composition, the coating may be designed to rupture and expose the core tablet at the desired location within the gastrointestinal tract. The film-coating process, which places a thin skin-tight coating of a plastic-like material over the compressed tablet, may produce coated tablets having essentially the same weight, shape, and size as the originally compressed tablet. The film-coating may be colored to make the tablets attractive and distinctive. Film-coating solutions may be non-aqueous or aqueous. In particular embodiments, the non-aqueous solutions may optionally contain one or more of the following types of materials to provide the desired coating to the tablets: (1) a film former capable of producing smooth, thin films reproducible under conventional coating conditions and applicable to a variety of tablet shapes, such as, for example, cellulose acetate phthalate; (2) an alloying substance providing water solubility or permeability to the film to ensure penetration by body fluids and therapeutic availability of the drug, such as, for example, polyethylene glycol; (3) a plasticizer to produce flexibility and elasticity of the coating and thus provide durability, such as, for example, castor oil; (4) a surfactant to enhance spreadability of the film during application, such as, for example, polyoxyethylene sorbitan derivatives; (5) opaquants and colorants to make the appearance of the coated tablets attractive and distinctive, such as, for example, titanium dioxide as an opaquant, and FD&C or D&C dyes as a colorant; (6) sweeteners, flavors, or aromas to enhance the acceptability of the tablet to the subject, such as, for example, saccharin as sweeteners, and vanillin as flavors and aromas; (7) a glossant to provide a luster to the tablets without a separate polishing operation, such as, for example, beeswax; and (8) a volatile solvent to allow the spread of the other components over the tablets while allowing rapid evaporation to permit an effective yet speedy operation, such as, for example, alcohol-acetone mixture. In certain embodiments, an aqueous film-coating formulation may contain one or more of the following: (1) film-forming polymer, such as, for example, cellulose ether polymers as hydroxypropyl methyl-cellulose, hydroxypropyl cellulose, and methyl-cellulose; (2) plasticizer, such as, for example, glycerin, propylene glycol, polyethylene glycol, diethyl phthalate, and dibutyl subacetate; (3) colorant and opacifier, such as, for example, FD&C or D&C lakes and iron oxide pigments; or (4) vehicle, such as, for example, water.

In certain embodiments, the compressed tablet of a cytidine analog may be compression-coated. The coating material, in the form of a granulation or powder, may be compressed onto a tablet core of drug with a special tablet press.

In certain embodiments, the pharmaceutical formulation is a gelatin-coated tablet of a cytidine analog. A gelatin-coated tablet is a capsule-shaped compressed tablet that allows the coated product to be smaller than a capsule filled with an equivalent amount of powder. The gelatin coating facilitates swallowing and compared to unsealed capsules, gelatin-coated tablets may be more tamper-evident.

In certain embodiments, the pharmaceutical formulation may be a sublingual tablet of a cytidine analog. The sublingual tablet is intended to be dissolved beneath the tongue for absorption through the oral mucosa. The sublingual tablet may dissolve promptly and provide rapid release of the drug.

In certain embodiments, the pharmaceutical formulation is an immediate release tablet of a cytidine analog. In certain embodiments, the immediate release tablet is designed, e.g., to disintegrate and release the API absent of any special rate-controlling features, such as special coatings and other techniques. In certain embodiments, the formulation is a rapidly disintegrating tablet that, e.g., dissolves substantially in the mouth following administration. In certain embodiments, the pharmaceutical formulation is an extended release tablet of a cytidine analog. In certain embodiments, the extended release tablet is designed, e.g., to release the API over an extended period of time and substantially in the stomach.

In certain embodiments, compressed tablets may be prepared by wet granulation. Wet granulation is a widely employed method for the production of compressed tablets, and, in particular embodiments, requires one or more the following steps: (1) weighing and blending the ingredients; (2) preparing a damp mass; (3) screening the damp mass into pellets or granules; (4) drying the granulation; (5) sizing the granulation by dry screening; (6) adding lubricant and blending; and (7) tableting by compression.

In certain embodiments, compressed tablets may be prepared by dry granulation. By the dry granulation method, the powder mixture is compacted in large pieces and subsequently broken down or sized into granules. But this method, either the active ingredient or the diluent has cohesive property. After weighing and mixing the ingredients, the powder mixture may be slugged or compressed into large flat tablets or pellets. The slugs then are broken up by hand or by a mill and passed through a screen of desired mesh for sizing. Lubricant is added in the usual manner, and tablets are prepared by compression. Alternatively, instead of slugging, powder compactors may be used to increase the density of a powder by pressing it between high-pressure rollers. The compressed material then is broken up, sized, and lubricated, and tablets are prepared by compression in the usual manner. The roller compaction method is often preferred over slugging. Binding agents used in roller compaction formulations include methylcellulose or hydroxyl-methylcellulose and can produce good tablet hardness and friability.

In certain embodiments, compressed tablets may be prepared by direct compression. Some granular chemicals possess free flowing and cohesive properties that enable them to be compressed directly in a tablet machine without the need of wet or dry granulation. For chemicals that do not possess this quality, special pharmaceutical excipients may be used which impart the necessary qualities for the production of tablets by direct compression. Particular tableting excipients include, e.g.: fillers, such as spray-dried lactose, micro-crystals of alpha-monohydrate lactose, sucrose-invert sugar-corn starch mixtures, micro-crystalline cellulose, crystalline maltose, and di-calcium phosphate; disintegrating agents, such as direct-compression starch, sodium carboxymethyl starch, cross-linked carboxymethylcellulose fibers, and cross-linked polyvinylpyrrolidone; lubricants, such as magnesium searate and talc; and glidants, such as fumed silicon dioxide.

In certain embodiments, tablets provided herein may be prepared by molding. The base for molded tablets is generally a mixture of finely powdered lactose with or without a portion of powdered sucrose. In preparing the fill, the drug is mixed uniformly with the base by geometric dilution. The powder mixture may be wetted with a mixture of water and alcohol sufficient only to dampen the powder so that it may be compacted. The solvent action of the water on a portion of the lactose/sucrose base effects the biding of the powder mixture upon drying. The alcohol portion hastens the drying process.

In certain embodiments, the pharmaceutical formulations provided herein contain the cytidine analog and, optionally, one or more excipients to form a “drug core.” Optional excipients include, e.g., diluents (bulking agents), lubricants, disintegrants, fillers, stabilizers, surfactants, preservatives, coloring agents, flavoring agents, binding agents, excipient supports, glidants, permeation enhancement excipients, plasticizers and the like, e.g., as known in the art. It will be understood by those in the art that some substances serve more than one purpose in a pharmaceutical composition. For instance, some substances are binders that help hold a tablet together after compression, yet are also disintegrants that help break the tablet apart once it reaches the target delivery site. Selection of excipients and amounts to use may be readily determined by the formulation scientist based upon experience and consideration of standard procedures and reference works available in the art.

In certain embodiments, formulations provided herein comprise one or more binders. Binders may be used, e.g., to impart cohesive qualities to a tablet, and thus ensure that the tablet remains intact after compression. Suitable binders include, but are not limited to, starch (including corn starch and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropylmethylcellulose, methyl cellulose, ethyl cellulose, hydroxyethyl cellulose, carboxymethyl cellulose and the like), veegum, carbomer (e.g., carbopol), sodium, dextrin, guar gum, hydrogenated vegetable oil, magnesium aluminum silicate, maltodextrin, polymethacrylates, povidone (e.g., KOLLIDON, PLASDONE), microcrystalline cellulose, among others. Binding agents also include, e.g., acacia, agar, alginic acid, cabomers, carrageenan, cellulose acetate phthalate, ceratonia, chitosan, confectioner's sugar, copovidone, dextrates, dextrin, dextrose, ethylcellulose, gelatin, glyceryl behenate, guar gum, hydroxyethyl cellulose, hydroxyethylmethyl cellulose, hydroxypropyl cellulose, hydroxypropyl starch, hypromellose, inulin, lactose, magnesium aluminum silicate, maltodextrin, maltose, methylcellulose, poloxamer, polycarbophil, polydextrose, polyethylene oxide, polymethylacrylates, povidone, sodium alginate, sodium carboxymethylcellulose, starch, pregelatinized starch, stearic acid, sucrose, and zein. The binding agent can be, relative to the drug core, in the amount of about 2% w/w of the drug core; about 4% w/w of the drug core, about 6% w/w of the drug core, about 8% w/w of the drug core, about 10% w/w of the drug core, about 12% w/w of the drug core, about 14% w/w of the drug core, about 16% w/w of the drug core, about 18% w/w of the drug core, about 20% w/w of the drug core, about 22% w/w of the drug core, about 24% w/w of the drug core, about 26% w/w of the drug core, about 28% w/w of the drug core, about 30% w/w of the drug core, about 32%/w/w of the drug core, about 34% w/w of the drug core, about 36% w/w of the drug core, about 38% w/w of the drug core, about 40% w/w of the drug core, about 42% w/w of the drug core, about 44% w/w of the drug core, about 46% w/w of the drug core, about 48% w/w of the drug core, about 50% w/w of the drug core, about 52% w/w of the drug core, about 54% w/w of the drug core, about 56% w/w of the drug core, about 58% w/w of the drug core, about 60% w/w of the drug core, about 62% w/w of the drug core, about 64% w/w of the drug core, about 66% w/w of the drug core; about 68% w/w of the drug core, about 70% w/w of the drug core, about 72% w/w of the drug core, about 74% w/w of the drug core, about 76% w/w of the drug core, about 78% w/w of the drug core, about 80%/w/w of the drug core, about 82% w/w of the drug core, about 84% w/w of the drug core, about 86% w/w of the drug core, about 88% w/w of the drug core, about 90% w/w of the drug core, about 92% w/w of the drug core, about 94% w/w of the drug core, about 96% w/w of the drug core, about 98% w/w of the drug core, or more, if determined to be appropriate. In certain embodiments, a suitable amount of a particular binder is determined by one of ordinary skill in the art.

In certain embodiments, formulations provided herein comprise one or more diluents. Diluents may be used, e.g., to increase bulk so that a practical size tablet is ultimately provided. Suitable diluents include dicalcium phosphate, calcium sulfate, lactose, cellulose, kaolin, mannitol, sodium chloride, dry starch, microcrystalline cellulose (e.g., AVICEL), microfine cellulose, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT), potassium chloride, sodium chloride, sorbitol and talc, among others. Diluents also include, e.g., ammonium alginate, calcium carbonate, calcium phosphate, calcium sulfate, cellulose acetate, compressible sugar, confectioner's sugar, dextrates, dextrin, dextrose, erythritol, ethylcellulose, fructose, fumaric acid, glyceryl palmitostearate, isomalt, kaolin, lacitol, lactose, mannitol, magnesium carbonate, magnesium oxide, maltodextrin, maltose, medium-chain triglycerides, microcrystalline cellulose, microcrystalline silicified cellulose, powered cellulose, polydextrose, polymethylacrylates, simethicone, sodium alginate, sodium chloride, sorbitol, starch, pregelatinized starch, sucrose, sulfobutylether-R-cyclodextrin, talc, tragacanth, trehalose, and xylitol. Diluents may be used in amounts calculated to obtain a desired volume for a tablet or capsule; in certain embodiments, a diluent is used in an amount of about 5% or more, about 10% or more, about 15% or more, about 20% or more, about 22% or more, about 24% or more, about 26% or more, about 28% or more, about 30% or more, about 32% or more, about 34% or more, about 36% or more, about 38% or more, about 40% or more, about 42% or more, about 44% or more, about 46% or more, about 48% or more, about 50% or more, about 52% or more, about 54% or more, about 56% or more, about 58% or more, about 60% or more, about 62% or more, about 64% or more, about 68% or more, about 70% or more, about 72% or more, about 74% or more, about 76% or more, about 78% or more, about 80% or more, about 85% or more, about 90% or more, or about 95% or more, weight/weight, of a drug core; between about 10% and about 90% w/w of the drug core; between about 20% and about 80% w/w of the drug core; between about 30% and about 70% w/w of the drug core; between about 40% and about 60% w/w of the drug core. In certain embodiments, a suitable amount of a particular diluent is determined by one of ordinary skill in the art.

In certain embodiments, formulations provided herein comprise one or more lubricants. Lubricants may be used, e.g., to facilitate tablet manufacture; examples of suitable lubricants include, for example, vegetable oils such as peanut oil, cottonseed oil, sesame oil, olive oil, corn oil, and oil of theobroma, glycerin, magnesium stearate, calcium stearate, and stearic acid. In certain embodiments, stearates, if present, represent no more than approximately 2 weight % of the drug-containing core. Further examples of lubricants include, e.g., calcium stearate, glycerin monostearate, glyceryl behenate, glyceryl palmitostearate, magnesium lauryl sulfate, magnesium stearate, myristic acid, palmitic acid, poloxamer, polyethylene glycol, potassium benzoate, sodium benzoate, sodium chloride, sodium lauryl sulfate, sodium stearyl fumarate, stearic acid, talc, and zinc stearate. In particular embodiments, the lubricant is magnesium stearate. In certain embodiments, the lubricant is present, relative to the drug core, in an amount of about 0.2% w/w of the drug core, about 0.4% w/w of the drug core, about 0.6% w/w of the drug core, about 0.8% w/w of the drug core, about 1.0% w/w of the drug core, about 1.2% w/w of the drug core, about 1.4% w/w of the drug core, about 1.6% w/w of the drug core, about 1.8% w/w of the drug core, about 2.0% w/w of the drug core, about 2.2% w/w of the drug core, about 2.4% w/w of the drug core, about 2.6% w/w of the drug core, about 2.8% w/w of the drug core, about 3.0% w/w of the drug core, about 3.5% w/w of the drug core, about 4% w/w of the drug core, about 4.5% w/w of the drug core, about 5% w/w of the drug core, about 6% w/w of the drug core, about 7% w/w of the drug core, about 8% w/w of the drug core, about 10% w/w of the drug core, about 12% w/w of the drug core, about 14% w/w of the drug core, about 16% w/w of the drug core, about 18% w/w of the drug core, about 20% w/w of the drug core, about 25% w/w of the drug core, about 30% w/w of the drug core, about 35% w/w of the drug core, about 40% w/w of the drug core, between about 0.2% and about 10% w/w of the drug core, between about 0.5% and about 5% w/w of the drug core, or between about 1% and about 3% w/w of the drug core. In certain embodiments, a suitable amount of a particular lubricant is determined by one of ordinary skill in the art.

In certain embodiments, formulations provided herein comprise one or more disintegrants. Disintegrants may be used, e.g., to facilitate disintegration of the tablet, and may be, e.g., starches, clays, celluloses, algins, gums or crosslinked polymers. Disintegrants also include, e.g., alginic acid, carboxymethylcellulose calcium, carboxymethylcellulose sodium (e.g., AC-DI-SOL, PRIMELLOSE), colloidal silicon dioxide, croscarmellose sodium, crospovidone (e.g., KOLLIDON, POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB) and starch. Additional disintegrants include, e.g., calcium alginate, chitosan, sodium docusate, hydroxypropyl cellulose, and povidone. In certain embodiments, the disintegrant is, relative to the drug core, present in the amount of about 1% w/w of the drug core, about 2% w/w of the drug core, about 3% w/w of the drug core, about 4% w/w of the drug core, about 5% w/w of the drug core, about 6% w/w of the drug core, about 7% w/w of the drug core, about 8% w/w of the drug core, about 9% w/w of the drug core, about 10% w/w of the drug core, about 12% w/w of the drug core, about 14% w/w of the drug core, about 16% w/w of the drug core, about 18% w/w of the drug core, about 20% w/w of the drug core, about 22% w/w of the drug core, about 24% w/w of the drug core, about 26% w/w of the drug core, about 28% w/w of the drug core, about 30% w/w of the drug core, about 32% w/w of the drug core, greater than about 32% w/w of the drug core, between about 1% and about 10% w/w of the drug core, between about 2% and about 8% w/w of the drug core, between about 3% and about 7% w/w of the drug core, or between about 4% and about 6% w/w of the drug core. In certain embodiments, a suitable amount of a particular disintegrant is determined by one of ordinary skill in the art.

In certain embodiments, formulations provided herein comprise one or more stabilizers. Stabilizers (also called absorption enhancers) may be used, e.g., to inhibit or retard drug decomposition reactions that include, by way of example, oxidative reactions. Stabilizing agents include, e.g., d-Alpha-tocopheryl polyethylene glycol 1000 succinate (Vitamin E TPGS), acacia, albumin, alginic acid, aluminum stearate, ammonium alginate, ascorbic acid, ascorbyl palmitate, bentonite, butylated hydroxytoluene, calcium alginate, calcium stearate, calcium carboxymethylcellulose, carrageenan, ceratonia, colloidal silicon dioxide, cyclodextrins, diethanolamine, edetates, ethylcellulose, ethyleneglycol palmitostearate, glycerin monostearate, guar gum, hydroxypropyl cellulose, hypromellose, invert sugar, lecithin, magnesium aluminum silicate, monoethanolamine, pectin, poloxamer, polyvinyl alcohol, potassium alginate, potassium polacrilin, povidone, propyl gallate, propylene glycol, propylene glycol alginate, raffinose, sodium acetate, sodium alginate, sodium borate, sodium carboxymethyl cellulose, sodium stearyl fumarate, sorbitol, stearyl alcohol, sufobutyl-b-cyclodextrin, trehalose, white wax, xanthan gum, xylitol, yellow wax, and zinc acetate. In certain embodiments, the stabilizer is, relative to the drug core, present in the amount of about 1% w/w of the drug core, about 2% w/w of the drug core, about 3% w/w of the drug core, about 4% w/w of the drug core, about 5% w/w of the drug core, about 6% w/w of the drug core, about 7% w/w of the drug core, about 8% w/w of the drug core, about 9% w/w of the drug core, about 10% w/w of the drug core, about 12% w/w of the drug core, about 14% w/w of the drug core, about 16% w/w of the drug core, about 18% w/w of the drug core, about 20% w/w of the drug core, about 22% w/w of the drug core, about 24% w/w of the drug core, about 26% w/w of the drug core, about 28% w/w of the drug core, about 30% w/w of the drug core, about 32% w/w of the drug core, between about 1% and about 10% w/w of the drug core, between about 2% and about 8% w/w of the drug core, between about 3% and about 7% w/w of the drug core, or between about 4% and about 6% w/w of the drug core. In certain embodiments, a suitable amount of a particular stabilizer is determined by one of ordinary skill in the art.

In certain embodiments, formulations provided herein comprise one or more glidants. Glidants may be used, e.g., to improve the flow properties of a powder composition or granulate or to improve the accuracy of dosing. Excipients that may function as glidants include, e.g., colloidal silicon dioxide, magnesium trisilicate, powdered cellulose, starch, tribasic calcium phosphate, calcium silicate, powdered cellulose, colloidal silicon dioxide, magnesium silicate, magnesium trisilicate, silicon dioxide, starch, tribasic calcium phosphate, and talc. In certain embodiments, the glidant is, relative to the drug core, present in the amount of less than about 1% w/w of the drug core, about 1% w/w of the drug core, about 2% w/w of the drug core, about 3% w/w of the drug core, about 4% w/w of the drug core, about 5% w/w of the drug core, about 6% w/w of the drug core, about 7% w/w of the drug core, about 8% w/w of the drug core, about 9% w/w of the drug core, about 10% w/w of the drug core, about 12% w/w of the drug core, about 14% w/w of the drug core, about 16% w/w of the drug core, about 18% w/w of the drug core, about 20% w/w of the drug core, about 22% w/w of the drug core, about 24% w/w of the drug core, about 26% w/w of the drug core, about 28% w/w of the drug core, about 30% w/w of the drug core, about 32% w/w of the drug core, between about 1% and about 10% w/w of the drug core, between about 2% and about 8% w/w of the drug core, between about 3% and about 7% w/w of the drug core, or between about 4% and about 6% w/w of the drug core. In certain embodiments, a suitable amount of a particular glidant is determined by one of ordinary skill in the art.

In certain embodiments, formulations provided herein comprise one or more permeation enhancers (also called, e.g., permeability enhancers). In certain embodiments, the permeation enhancer enhances the uptake of a cytidine analog through the gastrointestinal wall (e.g., the stomach). In certain embodiments, the permeation enhancer alters the rate and/or amount of the cytidine analog that enters the bloodstream. In particular embodiments, d-alpha-tocopheryl polyethylene glycol-1000 succinate (Vitamin E TPGS) is used as a permeation enhancer. In particular embodiments, one or more other suitable permeation enhancers are used, including, e.g., any permeation enhancer known in the art. Specific examples of suitable permeation enhancers include, e.g., those listed below:

| | | Example of |
|---|---|---|
| Product name | Chemical Name | Supplier |
| Pluronic F 127 | Poloxamer F 127 | Sigma |
| Lutrol F 68 | Poloxamer 188 | BASF |
| Carbopol 934-P | Carbomer 934-P | Spectrum |
| | | Chemical |
| Tween 80 | Polysorbate 80 | Sigma |
| Chitosan | Chitosan Low Mol Wt | Aldrich |
| Capric acid/Na cap | Sodium Decanoate | Sigma |
| Lauric acid/Na laur | Sodium Dodecanoate | Sigma |
| Disodium EDTA | Ethylenediamine tetraacetic | Sigma |
| | acid disodium dehydrate | |
| Propylene glycol | 1,2 Propanediol | Sigma |
| CM Cellulose | Carboxymethyl Cellulose | Sigma |
| Labrasol | Caprylocaproyl macrogol-8 | Gattefosse |
| | glycerides | |
| N,N- Dimethylacetamide | (minimum 99%) | Sigma |
| Vitamin E TPGS | d-Alpha-Tocopheryl | Eastman |
| | Polyethylene Glycol-1000 | |
| | Succinate | |
| Solutol HS 15 | Polyethylene glycol 660 | BASF |
| | 12-hydroxystearate | |
| Labrafil M 1944 CS (2) | Oleyl Macrogolglyerides | Gattefosse |

Other potential permeation enhancers include, e.g., alcohols, dimethyl sulfoxide, glyceryl monooleate, glycofurol, isopropyl myristate, isopropyl palmitate, lanolin, linoleic acid, myristic acid, oleic acid, oleyl alcohol, palmitic acid, polyoxyethylene alkyl ethers, 2-pyrrolidone, sodium lauryl sulfate, and thymol.

In certain embodiments, the permeation enhancer is present in the formulation in an amount by weight, relative to the total weight of the formulation, of about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1% about 4.2%, about 4.3%, about 4.4%, about 4.5%, about 4.6%, about 4.7%, about 4.8%, about 4.9%, about 5%, about 5.1% about 5.2%, about 5.3%, about 5.4%, about 5.5%, about 5.6%, about 5.7%, about 5.8%, about 5.9%, about 6%, about 6.1% about 6.2%, about 6.3%, about 6.4%, about 6.5%, about 6.6%, about 6.7%, about 6.8%, about 6.9%, about 7%, about 7.1% about 7.2%, about 7.3%, about 7.4%, about 7.5%, about 7.6%, about 7.7%, about 7.8%, about 7.9%, about 8%, about 8.1% about 8.2%, about 8.3%, about 8.4%, about 8.5%, about 8.6%, about 8.7%, about 8.8%, about 8.9%, about 9%, about 9.1% about 9.2%, about 9.3%, about 9.4%, about 9.5%, about 9.6%, about 9.7%, about 9.8%, about 9.9%, about 10%, greater than about 10%, greater than about 12%, greater than about 14%, greater than about 16%, greater than about 18%, greater than about 20%, greater than about 25%, greater than about 30%, greater than about 35%, greater than about 40%, greater than about 45%, or greater than about 50%. In certain embodiments, the appropriate amount of a suitable permeation enhancer provided herein is determined by one of skill in the art.

Without intending to be limited to any particular theory, the permeation enhancers provided herein may function by, inter alia, facilitating (e.g., increasing the rate or extent of) the transport of a cytidine analog through the gastrointestinal wall. In general, movement through the gastrointestinal wall may occur by, e.g.: passive diffusion, such as the movement of drug across a membrane in a manner driven solely by the concentration gradient; carrier-mediated diffusion, such as the movement of drug across a cell membrane via a specialized transport system embedded in the cell membrane; paracellular diffusion, such as the movement of a drug across a membrane by going between, rather than through, two cells; and transcellular diffusion, such as the movement of a drug across the cell. Additionally, there are numerous cellular proteins capable of preventing intracellular accumulation of drugs by pumping out drug that enters the cell. These are sometimes called efflux pumps. One such efflux pump is that involving p-glycoprotein, which is present in many different tissues in the body (e.g., intestine, placental membrane, blood-brain barrier). Permeation enhancers can function by, inter alia, facilitating any of the processes mentioned above (such as by increasing fluidity of membranes, opening tight junctions between cells, and/or inhibiting efflux, among others).

In certain embodiments, the compositions provided herein comprising a cytidine analog, e.g., 5-azacytidine, are essentially free of a cytidine deaminase inhibitor (e.g., do not comprise a cytidine deaminase inhibitor). In certain embodiments, the compositions provided herein are essentially free of (e.g., do not comprise) the cytidine deaminase inhibitor tetrahydrouridine (THU). Certain embodiments herein provide pharmaceutical compositions comprising a therapeutically effective amount of a cytidine analog (e.g., 5-azacytidine), wherein the compositions release the cytidine analog substantially in the stomach following oral administration to a subject, and wherein the compositions are essentially free of (e.g., do not comprise) a cytidine deaminase inhibitor (e.g., THU). Certain embodiments herein provide pharmaceutical compositions comprising a therapeutically effective amount of a cytidine analog (e.g., 5-azacytidine), wherein the compositions release the cytidine analog substantially in the stomach following oral administration to a subject, wherein the compositions are essentially free of (e.g., do not comprise) a cytidine deaminase inhibitor (e.g., THU), and wherein the compositions achieve a particular biological parameter provided herein (e.g., a particular Cmax value. Tmax value, and/or AUC value provided herein). In particular embodiments, a composition provided herein that is essentially free of a cytidine deaminase inhibitor (e.g., THU) comprises, e.g., less than 200 mg, less than 150 mg, less than 100 mg, less than 50 mg, less than 25 mg, less than 10 mg, less than 5 mg, less than 1 mg, or less than 0.1 mg of the cytidine deaminase inhibitor.

4. Additional Therapeutic Agents

In particular embodiments, the cytidine analog oral formulations provided herein further comprise one, two, three, or more other pharmacologically active substances (also termed herein “additional therapeutic agents,” “second active agents,” or the like). In particular embodiments, the oral formulations provided herein comprise the additional therapeutic agent(s) in a therapeutically effective amount. In particular embodiments, the cytidine analog (e.g., 5-azacytidine) and the additional therapeutic agent(s) are co-formulated together in the same dosage form using methods of co-formulating active pharmaceutical ingredients, including methods disclosed herein and methods known in the art. In other embodiments, the cytidine analog and the additional therapeutic agent(s) are co-administered in separate dosage forms. It is believed that certain combinations work synergistically in the treatment of particular diseases or disorders, including, e.g., types of cancer and certain diseases and conditions associated with, or characterized by, undesired angiogenesis or abnormal cell proliferation. Cytidine analog oral dosage forms provided herein can also work to alleviate adverse effects associated with certain second active agents, and some second active agents can be used to alleviate adverse effects associated with cytidine analog oral dosage forms provided herein. In certain embodiments, the oral formulations provided herein are co-administered with one or more therapeutic agents to provide a resensitization effect in subjects in need thereof. Additional therapeutic agents can be, e.g., large molecules (e.g., proteins) or small molecules (e.g., synthetic inorganic, organometallic, or organic molecules). In a particular embodiment, the one or more additional therapeutic agent(s) include, but are not limited to, an anti-PD1/anti-PDL1 monoclonal antibody such as pembrolizumab and MEDI4736 (Durvalumab).

Examples of particular additional therapeutic agents useful in the compositions and methods disclosed herein include, but are not limited to, e.g., cytotoxic agents, anti-metabolites, antifolates, HDAC inhibitors (e.g., entinostat, also known as SNDX-275 or MS-275; or vorinostat, also known as suberoylanilide hydroxamic acid (SAHA) or N-hydroxy-N′-phenyl-octanediamide), DNA intercalating agents, DNA cross-linking agents, DNA alkylating agents, DNA cleaving agents, topoisomerase inhibitors, CDK inhibitors, JAK inhibitors, anti-angiogenic agents, Bcr-Abl inhibitors, HER2 inhibitors, EGFR inhibitors, VEGFR inhibitors, PDGFR inhibitors, HGFR inhibitors, IGFR inhibitors, c-Kit inhibitors, Ras pathway inhibitors, PI3K inhibitors, multi-targeted kinase inhibitors, mTOR inhibitors, anti-estrogens, anti-androgens, aromatase inhibitors, somatostatin analogs, ER modulators, anti-tubulin agents, vinca alkaloids, taxanes, HSP inhibitors, Smoothened antagonists, telomerase inhibitors, COX-2 inhibitors, anti-metastatic agents, immunosuppressants, biologics such as antibodies, and hormonal therapies. In particular embodiments, the co-administered therapeutic agent is an immunomodulatory compound, e.g., thalidomide, lenalidomide, or pomalidomide. The co-administered agent may be dosed, e.g., orally or by injection.

Other examples of additional therapeutic agents include, but are not limited to, hematopoietic growth factor, a cytokine, an anti-cancer agent, granulocyte colony-stimulating factor (G-CSF), granulocyte-macrophage colony-stimulating factor (GM-CSF), erythropoietin (EPO), interleukin (IL), interferon (IFN), oblimersen, melphalan, topotecan, pentoxifylline, taxotere, irinotecan, ciprofloxacin, doxorubicin, vincristine, dacarbazine, Ara-C, vinorelbine, prednisone, cyclophosphamide, bortezomib, arsenic trioxide. Such additional therapeutic agents are particularly useful in methods and compositions disclosed herein including, but not limited to, those relating to treatment of multiple myeloma.

Other examples of additional therapeutic agents include, but are not limited to, an antibody (e.g., rituximab, anti-CD33), hematopoietic growth factor, cytokine, anti-cancer agent, antibiotic, cox-2 inhibitor, immunomodulatory agent, immunosuppressive agent, corticosteroid, or a pharmacologically active mutant or derivative thereof. See, e.g., S. Nand et al., Leukemia and Lymphoma, 2008, 49(11):2141-47 (describing a Phase II study involving the administration of a combination of hydroxyurea, azacitidine and low dose gemtuzumab ozogamicin to elderly patients with AML and high-risk MDS, and concluding that this combination appears to be a safe and effective regimen in the treatment of AML and high risk MDS in this group of patients). Such additional therapeutic agents are particularly useful in methods and compositions disclosed herein including, but not limited to, those relating to treatment of the diseases and disorders disclosed herein.

Examples of large molecule active agents include, but are not limited to, hematopoietic growth factors, cytokines, and monoclonal and polyclonal antibodies. Typical large molecule active agents are biological molecules, such as naturally occurring or artificially made proteins. Proteins that are particularly useful include proteins that stimulate the survival and/or proliferation of hematopoietic precursor cells and immunologically active poietic cells in vitro or in vivo. Others stimulate the division and differentiation of committed crythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; and EPO.

Particular proteins that can be used in the methods and compositions provided herein include, but are not limited to: filgrastim, which is sold in the United States under the trade name Neupogen® (Amgen, Thousand Oaks, Calif.); sargramostim, which is sold in the United States under the trade name Leukine® (Immunex, Seattle, Wash.); and recombinant EPO, which is sold in the United States under the trade name Epogen® (Amgen, Thousand Oaks, Calif.).

Recombinant and mutated forms of GM-CSF can be prepared as described in U.S. Pat. Nos. 5,391,485; 5,393,870; and 5,229,496; all of which are incorporated herein by reference. Recombinant and mutated forms of G-CSF can be prepared as described in U.S. Pat. Nos. 4,810,643; 4,999,291; 5,528,823; and 5,580,755; all of which are incorporated herein by reference.

Embodiments herein encompass the use of native, naturally occurring, and recombinant proteins. Particular embodiments encompass mutants and derivatives (e.g., modified forms) of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins, such as proteins formed by fusing IgG1 or IgG3 to the protein or active portion of the protein of interest. See, e.g., Penichet, M. L. and Morrison, S. L., J. Immunol. Methods 248:91-101 (2001).

Antibodies that can be used in combination with oral formulations disclosed herein include monoclonal and polyclonal antibodies. Examples of antibodies include, but are not limited to, trastuzumab (Herceptin®), rituximab (Rituxan®), bevacizumab (Avastin™), pertuzumab (Omnitarg™), tositumomab (Bexxar®), edrecolomab (Panorex®), and G250. Oral formulations disclosed herein can also comprise, be combined with, or used in combination with anti-TNF-α antibodies. In a preferred embodiment, the antibody is an anti-PD1/anti-PDL1 monoclonal antibody such as pembrolizumab and MEDI4736 (Durvalumab).

Large molecule active agents may be administered in the form of anti-cancer vaccines. For example, vaccines that secrete, or cause the secretion of, cytokines such as IL-2, G-CSF, and GM-CSF can be used in the methods, pharmaceutical compositions, and kits provided herein. See, e.g., Emens, L. A., et al., Curr. Opinion Mol. Ther. 3(1):77-84 (2001).

In one embodiment, the additional therapeutic agent (e.g., large-molecule compound or small-molecule compound) reduces, eliminates, or prevents an adverse effect associated with the administration (e.g., oral administration) of a cytidine analog provided herein. Depending on the particular cytidine analog and the disease or disorder begin treated, adverse effects can include, but are not limited to, anemia, neutropenia, febrile neutropenia, thrombocytopenia, hepatotoxicity (e.g., including, but not limited to, hepatoxicity in patients with preexisting hepatic impairment), elevated serum creatinine, renal failure, renal tubular acidosis, hypokalemia, hepatic coma, nausea, vomiting, dyspepsia, abdominal pain, pyrexia, leukopenia, diarrhea, constipation, ecchymosis, petechiae, rigors, weakness, pneumonia, anxiety, insomnia, lethargy, and decrease in weight, among others known in the art to be associated with particular cytidine analogs.

Like some large molecules, many small-molecule compounds are believed to be capable of providing a synergistic effect when administered with (e.g., before, after or simultaneously) a cytidine analog oral formulation disclosed herein. Examples of small molecule second active agents include, but are not limited to, anti-cancer agents, antibiotics, immunosuppressive agents, and steroids.

Examples of anti-cancer agents include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur, epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O⁶-benzylguanine; octreotide; okicenone; oligonucleotides; onapristone; ondansetron; ondansetron; oracin; oral cytokine inducer, ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor, platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tel lurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Specific additional therapeutic agents include, but are not limited to, oblimersen (Genasense®), remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, Arisa®, taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, estramustine sodium phosphate (Emcyt®), sulindac, and etoposide.

### D. Methods of Use

The combination of 5-azacytidine, or a pharmaceutically acceptable salt, solvate or hydrate thereof with one or more additional therapeutic agent(s), including an anti-PD or anti-PDL1 monoclonal antibody, provided herein can be used in all the methods as provided herein. Particularly, the combination of 5-azacytidine, or a pharmaceutically acceptable salt, solvate or hydrate thereof with one or more additional therapeutic agent(s), including an anti-PD1 or anti-PDL1 monoclonal antibody provided herein can be used in the treatment, prevention or improvement of all diseases disorders, or conditions provided herein.

In one embodiment, a method for treating a subject having a disease or disorder, wherein the method comprises cyclically administering to the subject a therapeutically effective amount of a cytidine analog (e.g., 5-azacytidine), or a pharmaceutically acceptable salt, solvate or hydrate thereof, and a therapeutically effective amount of one or more therapeutic agent(s), including an anti-PD1/anti-PDL1 monoclonal antibody, wherein the 5-azacytidine, or a pharmaceutically acceptable salt, solvate or hydrate thereof is administered orally.

In one embodiment, the disease or disorder is a solid tumor.

In one embodiment, the disease or disorder is a hematological disorder.

In one embodiment, the disease or disorder is myelodysplastic syndromes, acute myeloid leukemia, ovarian cancer, or non-small cell lung cancer.

In one embodiment, the disease or disorder is relapsed or refractory.

In one embodiment, the subject having a disease or disorder did not respond to a prior treatment.

In one embodiment, the prior treatment comprises an injectable hypomethylating agent.

In one embodiment, the prior treatment comprises a platinum based regimen.

In one embodiment, the ovarian cancer is epithelial ovarian cancer.

In one embodiment, the epithelial ovarian cancer is relapsed epithelial ovarian cancer.

In one embodiment, the relapsed or refractory epithelial ovarian cancer followed a prior treatment comprising a platinum based regimen.

In one embodiment, the relapsed or refractory non-small cell lung cancer followed a prior treatment comprising a platinum based regimen.

In one embodiment, the relapsed or refractory epithelial ovarian cancer followed a prior treatment comprising an injectable hypomethylating agent.

In one embodiment, the relapsed or refractory non-small cell lung cancer followed a prior treatment comprising an injectable hypomethylating agent.

In one embodiment, the relapsed or refractory myelodysplastic syndromes followed a prior treatment comprising an injectable hypomethylating agent.

In one embodiment, the relapsed or refractory acute myeloid leukemia followed a prior treatment comprising an injectable hypomethylating agent.

In one embodiment, the relapsed or refractory myelodysplastic syndromes followed a prior treatment comprising a platinum based regimen.

In one embodiment, relapsed or refractory acute myeloid leukemia followed a prior treatment comprising a platinum based regimen.

In one embodiment, the anti-PD1 monoclonal antibody is a humanized monoclonal IgG4 antibody.

In one embodiment, the anti-PDL1 monoclonal antibody is a humanized monoclonal IgG1 antibody.

In one embodiment, the humanized monoclonal IgG4 antibody is pembrolizumab, MK-3475, pidilizumab, Nivolumab (BMS-936558, MDX-1106, or ONO-4538).

In one embodiment, the humanized monoclonal IgG4 antibody is pembrolizumab.

In one embodiment, the humanized monoclonal IgG1 antibody is BMS-936559, atezolizumab (MPDL3280A), or durvalumab (MEDI4736).

In one embodiment, the humanized monoclonal IgG1 antibody is durvalumab (MEDI4736).

In one embodiment, 5-azacytidine, or a pharmaceutically acceptable salt, solvate or hydrate thereof is administered for 21 consecutive days followed by seven consecutive days of rest in a 28 day cycle.

In one embodiment, 5-azacytidine, or a pharmaceutically acceptable salt, solvate or hydrate thereof is administered for 14 consecutive days followed by seven consecutive days of rest in a 21 day cycle.

In certain embodiments, 5-azacytidine, or a pharmaceutically acceptable salt, solvate or hydrate thereof is administered day 1 through day 7, every 4 weeks (Q4W). 5-azacytidine will be administered at 75 mg/m²/day, day 1 through day 7, every 4 weeks (Q4W).

In one embodiment, the anti-PD1/anti-PDL1 monoclonal antibody is administered on days 7 and 21 in a 28 day cycle.

In one embodiment, the anti-PD1/anti-PDL1 monoclonal antibody is administered on day 1 in a 28 day cycle.

In one embodiment, the anti-PD1/anti-PDL1 monoclonal antibody is administered on days 8 and 21 in a 28 day cycle.

In one embodiment, the anti-PD1/anti-PDL1 monoclonal antibody is administered on day 1 in a 21 day cycle.

In one embodiment, the anti-PD1/anti-PDL1 monoclonal antibody is administered on day 1 in a 14 day cycle.

In one embodiment, 5-azacytidine, or a pharmaceutically acceptable salt, solvate or hydrate thereof is administered for 21 consecutive days followed by seven consecutive days of rest in a 28 day cycle, and the anti-PD1/anti-PDL1 monoclonal antibody is administered on day 1 of the 28 day cycle. The disease or disorder is MDS or AML (e.g., relapsed or refractory MDS or AML, and, more particularly, MDS or AML that is not responding to treatment with injectable hypomethylating agents).

In one embodiment, 5-azacytidine, or a pharmaceutically acceptable salt, solvate or hydrate thereof is administered for 21 consecutive days followed by seven consecutive days of rest in a 28 day cycle, and the anti-PD1/anti-PDL1 monoclonal antibody is administered on days 7 and 21 of the 28 day cycle. The disease or disorder is MDS or AML (e.g., relapsed or refractory MDS or AML, and, more particularly, MDS or AML that is not responding to treatment with injectable hypomethylating agents).

In one embodiment, 5-azacytidine, or a pharmaceutically acceptable salt, solvate or hydrate thereof is administered for 21 consecutive days followed by seven consecutive days of rest in a 28 day cycle, and the anti-PD1/anti-PDL1 monoclonal antibody is administered on days 8 and 21 of the 28 day cycle. The disease or disorder is MDS or AML (e.g., relapsed or refractory MDS or AML, and, more particularly, MDS or AML that is not responding to treatment with injectable hypomethylating agents).

In one embodiment, 5-azacytidine, or a pharmaceutically acceptable salt, solvate or hydrate thereof is administered for 14 consecutive days followed by seven consecutive days of rest in a 21 day cycle, and the anti-PD1/anti-PDL1 monoclonal antibody is administered on day 1 of the 21 day cycle. The disease or disorder is ovarian cancer or lung cancer (e.g., epithelial ovarian cancer or non-small cell lung cancer, particularly, relapsed or refractory epithelial ovarian cancer or non-small cell lung cancer, and more particularly, epithelial ovarian cancer or non-small cell lung cancer relapsed following a platinum based therapy.

In one embodiment, 5-azacytidine, or a pharmaceutically acceptable salt, solvate or hydrate thereof is administered day 1 through day 7, every 4 weeks (Q4W) and the anti-PDL1 monoclonal antibody is administered every 2 weeks. The disease or disorder is MDS or AML (e.g., relapsed or refractory MDS or AML, and, more particularly, MDS or AML that is not responding to treatment with injectable hypomethylating agents or untreated (first-line) higher risk MDS).

In one embodiment, the 5-azacytidine, or a pharmaceutically acceptable salt, solvate or hydrate thereof is administered in an amount of about 300 mg per day for 14 consecutive days followed by seven consecutive days of rest in a 21 day cycle, and pembrolizumab is administered at a dose of 10 mg/kg on day 1 of the 21 day cycle, and the disease or disorder is relapsed epithelial ovarian cancer

In one embodiment, the 5-azacytidine, or a pharmaceutically acceptable salt, solvate or hydrate thereof is administered in an amount of about 100 mg twice per day, of about 150 mg twice per day, of about 200 mg twice per day or of about 300 mg twice per day for 21 consecutive days followed by seven consecutive days of rest in a 28 day cycle, and pembrolizumab is administered at a dose of 10 mg/kg on days 7 and 21 of the 28 day cycle, and the disease or disorder is acute myeloid leukemia (AML) not responding to treatment with injectable hypomethylating agents (HMAs) or myelodysplastic syndromes (MDS).

In one embodiment, the 5-azacytidine, or a pharmaceutically acceptable salt, solvate or hydrate thereof is administered in an amount of about 200 mg per day for 21 consecutive days followed by seven consecutive days of rest in a 28 day cycle, and pembrolizumab is administered at a dose of 10 mg/kg on days 7 and 21 of the 28 day cycle, and the disease or disorder is acute myeloid leukemia (AML) not responding to treatment with injectable hypomethylating agents (HMAs) or myelodysplastic syndromes (MDS).

In one embodiment, the 5-azacytidine, or a pharmaceutically acceptable salt, solvate or hydrate thereof is administered in an amount of about 200 mg per day for 21 consecutive days followed by seven consecutive days of rest in a 28 day cycle, and pembrolizumab is administered at a dose of 5 mg/kg on days 7 and 21 of the 28 day cycle, and the disease or disorder is acute myeloid leukemia (AML) not responding to treatment with injectable hypomethylating agents (HMAs) or myelodysplastic syndromes (MDS).

In one embodiment, the 5-azacytidine, or a pharmaceutically acceptable salt, solvate or hydrate thereof is administered in an amount of about 200 mg per day for 14 consecutive days followed by 14 consecutive days of rest in a 28 day cycle, and pembrolizumab is administered at a dose of 5 mg/kg on days 7 and 21 of the 28 day cycle, and the disease or disorder is acute myeloid leukemia (AML) not responding to treatment with injectable hypomethylating agents (HMAs) or myelodysplastic syndromes (MDS).

In one embodiment, the 5-azacytidine, or a pharmaceutically acceptable salt, solvate or hydrate thereof is administered in an amount of about 300 mg twice per day for 21 consecutive days followed by seven consecutive days of rest in a 28 day cycle, and pembrolizumab is administered at a dose of 10 mg/kg on days 8 and 21 of the 28 day cycle, and the disease or disorder is acute myeloid leukemia (AML) not responding to treatment with injectable hypomethylating agents (HMAs) or myelodysplastic syndromes (MDS).

In one embodiment, the 5-azacytidine, or a pharmaceutically acceptable salt, solvate or hydrate thereof is administered in an amount of about 300 mg per day for 14 consecutive days followed by seven consecutive days of rest in a 21 day cycle, and pembrolizumab is administered on day 1 of the 21 day cycle, and the disease or disorder is non-small cell lung cancer.

In one embodiment, the 5-azacytidine, or a pharmaceutically acceptable salt, solvate or hydrate thereof is administered in an amount of about 200 mg twice per day for 21 consecutive days followed by seven consecutive days of rest in a 28 day cycle, and durvalumab is administered at a dose of 1500 mg per day on days 7 and 21 of the 28 day cycle, or durvalumab is administered at a dose of 1500 mg per day on day 1 of the 28 day cycle, and the disease or disorder is acute myeloid leukemia (AML) or myelodysplastic syndromes (MDS) not responding to treatment with injectable hypomethylating agents (HMAs).

In one embodiment, the 5-azacytidine, or a pharmaceutically acceptable salt, solvate or hydrate thereof is administered in an amount of about 75 mg/m²/day for 7 consecutive days every 4 weeks (Q4W), and durvalumab is administered at a dose of 10 mg/kg BW every 2 weeks (Q2W), and the disease or disorder is acute myeloid leukemia (AML) or myelodysplastic syndromes (MDS) not responding to treatment with injectable hypomethylating agents (HMAs).

In one embodiment, 5-azacytidine, or a pharmaceutically acceptable salt, solvate or hydrate thereof is administered in an amount of about 50 mg, about 100 mg, about 200 mg, about 300 mg, about 400 mg, about 500 mg, or about 600 mg per day.

In one embodiment, 5-azacytidine, or a pharmaceutically acceptable salt, solvate or hydrate thereof is administered in an amount of about 300 mg per day.

In one embodiment, 5-azacytidine, or a pharmaceutically acceptable salt, solvate or hydrate thereof is administered in an amount of about 200 mg per day.

In one embodiment, 5-azacytidine, or a pharmaceutically acceptable salt, solvate or hydrate thereof, is administered once per day.

In one embodiment, 5-azacytidine, or a pharmaceutically acceptable salt, solvate or hydrate thereof, is administered twice per day.

In one embodiment, 5-azacytidine or a pharmaceutically acceptable salt, solvate or hydrate thereof is administered in an amount of about 200 mg, about 150 mg, or about 100 mg twice per day. In one embodiment, 5-azacytidine or a pharmaceutically acceptable salt, solvate or hydrate thereof is administered in an amount of about 200 mg, twice per day. In one embodiment, 5-azacytidine or a pharmaceutically acceptable salt, solvate or hydrate thereof is administered in an amount of about 150 mg twice per day. In one embodiment, 5-azacytidine or a pharmaceutically acceptable salt, solvate or hydrate thereof is administered in an amount of about 100 mg twice per day.

In one embodiment, the anti-PD1/anti-PDL1 monoclonal antibody is administered parenterally.

In one embodiment, the anti-PD1/anti-PDL1 monoclonal antibody is administered in an amount of about 0.5 mg of anti-PD1/anti-PDL1 monoclonal antibody per kilogram of a subject's mass, about 1 mg/Kg, about 2 mg/Kg, about 3 mg/Kg, about 4 mg/Kg, about 5 mg/Kg, about 6 mg/Kg, about 7 mg/Kg, about 8 mg/Kg, about 9 mg/Kg, about 10 mg/Kg, about 11 mg/Kg, about 12 mg/Kg, about 13 mg/Kg, about 14 mg/Kg, about 15 mg/Kg, about 16 mg/Kg, about 17 mg/Kg, about 18 mg/Kg, about 19 mg/Kg, or about 20 mg/Kg.

In one embodiment, the anti-PD1/anti-PDL1 monoclonal antibody is administered intravenously in an amount of about 10 mg/Kg per day.

In one embodiment, the anti-PD1/anti-PDL1 monoclonal antibody is administered intravenously in an amount of about 10 mg/Kg per day on days 7 and 21 in a 28 day cycle, or on days 8 and 21 in a 28 day cycle.

In one embodiment, the anti-PD1 monoclonal antibody is pembrolizumab, MK-3475, pidilizumab, Nivolumab (BMS-936558, MDX-1106, or ONO-4538) and is administered as a 30 minute i.v. infusion.

In one embodiment, the anti-PDL1 monoclonal antibody is BMS-936559, atezolizumab (MPDL3280A), or durvalumab (MEDI4736) and is administered as a 30 minute i.v. infusion. In one embodiment, 1,500 mg of durvalumab (MEDI4736) is administered on Day 1 of each 28-day treatment cycle by 1-hour intravenous (IV) infusion.

In one embodiment, 5-azacytidine or a pharmaceutically acceptable salt, solvate or hydrate thereof is in a form of a capsule, tablet or caplet.

In one embodiment, the method further comprises administering a therapeutically effective amount of an additional active agent.

In one embodiment, the subject is a human.

As described herein, certain embodiments herein provide oral formulations of cytidine analogs useful in methods relating to, e.g., permitting different dosing amounts and/or dosing periods; providing alternative pharmacokinetic profiles, pharmacodynamic profiles, and/or safety profiles; permitting the evaluation of long-term and/or maintenance therapies; providing treatment regimens that maximize demethylation and/or gene re-expression; providing treatment regimens that prolong continuous demethylation; providing new indications for cytidine analogs; and/or providing other potential advantageous benefits.

Provided herein are methods of treating patho-physiological conditions manifested by abnormal cell proliferation, such as, for example, cancer, including hematological disorders and solid tumors, by orally administering a pharmaceutical formulation comprising a cytidine analog, such as, for example, 5-azacytidine, wherein the formulation releases the cytidine analog substantially in the stomach. Other embodiments herein provide methods of treating immune disorders. In particular embodiments, the methods provided herein involve oral administering a formulation that effects an immediate release of the cytidine analog. In certain embodiments, the cytidine analog and one or more therapeutic agents are co-administered to subjects to yield a synergistic therapeutic effect. The co-administered agent may be a cancer therapeutic agent dosed orally or by injection.

In certain embodiments, methods provided herein for treating disorders related to abnormal cell proliferation comprise orally administering a formulation comprising a therapeutically effective amount of a cytidine analog. Particular therapeutic indications relating to the methods provided herein are disclosed herein. In certain embodiments, the therapeutically effective amount of the cytidine analog in the pharmaceutical formulation is an amount as disclosed herein. In certain embodiments, the precise therapeutically effective amount of the cytidine analog in the pharmaceutical formulation will vary depending on, e.g., the age, weight, disease and/or condition of the subject.

In particular embodiments, the disorders related to abnormal cell proliferation include, but are not limited to, MDS, AML, ALL, CML, leukemia, chronic lymphocytic leukemia (CLL), lymphoma (including non-Hodgkin's lymphoma (NHL) and Hodgkin's lymphoma), multiple myeloma (MM), sarcoma, melanoma, carcinoma, adenocarcinoma, chordoma, breast cancer, colorectal cancer, ovarian cancer, lung cancer (e.g., non-small-cell lung cancer and small-cell lung cancer), testicular cancer, renal cancer, pancreatic cancer, bone cancer, gastric cancer, head and neck cancer, and prostate cancer. In particular embodiment, the disorder related to abnormal cell proliferation is MDS. In particular embodiments, the disorder related to abnormal cell proliferation is AML.

In certain embodiments, methods provided herein for treating disorders of abnormal cell proliferation comprise administering a cytidine analog using at least two of IV, SC and oral administration methods. For example, particular embodiments herein provide administering an initial treatment cycle of a cytidine analog, such as, for example, 5-azacytidine, administered either SC or IV, followed by subsequent orally administered treatment cycles of the cytidine analog. In certain embodiments, treatment cycles comprise multiple doses administered to a subject in need thereof over multiple days (e.g., 1, 2, 3, 4, 5, 6, 7, 8, 9, 10, 11, 12, 13, 14, or greater than 14 days), optionally followed by treatment dosing holidays (e.g., 1, 2, 3, 4, 5, 6, 7, 8, 9, 10, 11, 12, 13, 14, or greater than 14 days). Particular embodiments herein provide a treatment schedule comprising SC and/or IV administration for one, two, three, four, five, or more initial cycles, followed by oral administration for subsequent cycles. For example, particular embodiments herein provide a treatment schedule comprising SC administration for cycle 1, followed by oral administration for subsequent cycles. Suitable dosage ranges and amounts for the methods provided herein are provided throughout the specification. For example, in certain embodiments, the SC dose is about 75 mg/m². In certain embodiments, the oral dose is about 60 mg, about 80 mg, about 120 mg, about 180 mg, about 240 mg, about 300 mg, about 360 mg, about 480 mg, or greater than about 480 mg. In certain embodiments, oral doses are calculated to achieve 80%, 100%, or 120% of SC AUC.

In certain embodiments, methods of treating disorders of abnormal cell proliferation comprises orally administering a formulation comprising a cytidine analog (e.g., 5-azacytidine) as single or multiple daily doses. In particular embodiments, the formulation(s) comprising the cytidine analog is/are orally administered once per day, twice per day, three times per day, four times per day, or more than four times per day. For example, in certain embodiments, the formulation comprising the cytidine analog is administered using a treatment cycle comprising administration of about 200 mg, about 300 mg, about 400 mg, about 500 mg, about 600 mg, about 700 mg, about 800 mg, about 900 mg, or about 1,000 mg of the cytidine analog once, twice, three, or four times per day for 7, 8, 9, 10, 11, 12, 13, 14, 15, 16, 17, 18, 19, 20, 21, 22, 23, 24, 25, 26, 27, 28, 29, or 30 days. In certain embodiments, the method of treating comprises continuous low-dose administration. In certain embodiments, the formulation comprising the cytidine analog is administered using a treatment cycle comprising administration of about 300 mg of the cytidine analog twice per day for 7 days. In certain embodiments, the formulation comprising the cytidine analog is administered using a treatment cycle comprising administration of about 300 mg of the cytidine analog twice per day for 14 days. In certain embodiments, the formulation comprising the cytidine analog is administered using a treatment cycle comprising administration of about 300 mg of the cytidine analog three times per day for 7 days. In certain embodiments, the formulation comprising the cytidine analog is administered using a treatment cycle comprising administration of about 300 mg of the cytidine analog three times per day for 14 days. In certain embodiments, methods provided herein comprise administering a formulation comprising a cytidine analog using one or more of the cycles provided herein, and repeating one or more of the cycles for a period of, e.g., 1, 2, 3, 4, 5, 6, 7, 8, 9, 10, 11, 12, or greater than 12 months.

In certain embodiments, methods herein comprise administering particular oral formulations provided herein to, e.g., overcome limitations associated with IV or SC administration of cytidine analogs. For example, IV or SC administration may limit the ability to deliver a cytidine analog for longer periods of time on a regular basis, thereby potentially limiting the maximal efficacy of the cytidine analog. Due to the difficulties of complying with the rigors of a prolonged IV or SC dosing schedule, prolonged SC or IV exposure to a cytidine analog may cause subjects (e.g., subjects with multiple cytopenias) to discontinue from the regimen. See, e.g., Lyons, R. M., et al., Hematologic Response to Three Alternative Dosing Schedules of Azacitidine in Patients With Myelodysplastic Syndromes, J. Clin. Oncol. (2009) (DOI:10.1200/JCO.2008.17.1058), which is incorporated by reference herein in its entirety. Accordingly, in certain embodiments, methods provided herein comprise administering an oral formulation provided herein to overcome these or other limitations associated with SC or IV cytidine analog administration. For example, in certain embodiments, methods provided herein comprise administering daily to a subject an oral formulation provided herein for 7 or more, 8 or more, 9 or more, 10 or more, 11 or more, 12 or more, 13 or more, 14 or more, 15 or more, 16 or more, 17 or more, 18 or more, 19 or more, 20 or more, or 21 or more days.

Certain embodiments herein provide methods comprising administering oral formulations of cytidine analogs provided herein comprising delivering the cytidine analog (e.g., azacitidine) at a lower dose over a more prolonged period of time, as compared to IV or SC administration. In particular embodiments, such methods comprise managing dose-related cytopenias (including, e.g., dose-related cytopenias associated with azacitidine) by administering an oral formulation provided herein. In certain embodiments, methods provided herein comprise administering an oral formulation provided herein to achieve an improved safety profile as compared to an IV or SC dose comprising the same cytidine analog.

As described herein, certain embodiments provide methods for improved treatment of particular diseases or disorders (e.g., treatment of solid tumors) by administering an oral formulation provided herein, as compared to IV or SC administration of the cytidine analog. In particular embodiments, certain methods herein provide administering oral formulations provided herein at lower doses for more prolonged periods of time, leading to improved demethylation. For example, certain methods provided herein comprise administering an oral formulation provided herein to treat a solid tumor while avoiding certain dose-limiting-toxicity-related side effects associated with dosing the cytidine analog via SC or IV administration. An example of certain toxicity-related drawbacks associated with administration of a cytidine analog are described, e.g., in K. Appleton et al., J. Clin. Oncol., Vol. 25(29):4603-4609 (2007), which is incorporated by reference herein in its entirety.

Particular embodiments herein provide methods for treating a subject having a disease or disorder provided herein by orally administering a pharmaceutical composition provided herein, wherein the treatment results in improved survival of the subject. In certain embodiments, the improved survival is measured as compared to one or more conventional care regimens. Particular embodiments herein provide methods for treating a subject having a disease or disorder provided herein by orally administering a pharmaceutical composition provided herein, wherein the treatment provides improved effectiveness. In particular embodiments, the improved effectiveness is measured using one or more endpoints for cancer clinical trials, as recommended by the U.S. Food and Drug Administration (FDA). For example, FDA provides Guidance for Industry on Clinical Trial Endpoints for the Approval of Cancer Drugs and Biologics (http/www.fda.gov/CbER/gdlns/clintrialend.htm). The FDA endpoints include, but are not limited to, Overall Survival, Endpoints Based on Tumor Assessments such as (i) Disease-Free Survival (ii) Objective Response Rate, (iii) Time to Progression and Progression-Free Survival and (iv) Time-to-Treatment Failure. Endpoints Involving Symptom Endpoints may include Specific Symptom Endpoints such as (i) Time to progression of cancer symptoms and (ii) A composite symptom endpoint. Biomarkers assayed from blood or body fluids may also be useful to determine the management of the disease.

In certain embodiments, the methods of treating disorders of abnormal cell proliferation comprise orally administering a formulation of a cytidine analog with food. In certain embodiments, the methods of treating disorders of abnormal cell proliferation comprise orally administering a formulation of a cytidine analog without food. In certain embodiments, pharmacological parameters (e.g., Cmax, Tmax) depend on the fed state of the subject. In certain embodiments, the formulation of the cytidine analog is administered sublingually.

In certain embodiments, the cytidine analog, e.g., 5-azacytidine, is not co-administered with a cytidine deaminase inhibitor. In certain embodiments, the oral formulation comprising a cytidine analog as provided herein is not co-administered with THU. Certain embodiments herein provide methods of treating a disease or disorder provided herein (e.g., a disease associated with abnormal cell proliferation) comprising orally administering a cytidine analog provided herein (e.g., 5-azacytidine) for release substantially in the stomach, wherein the methods achieve a particular biological parameter provided herein (e.g., a particular Cₘₐₓ (value, Tₘₐₓ value, and/or AUC value provided herein), and wherein the methods comprise not co-administering a cytidine deaminase inhibitor with the cytidine analog. Certain embodiments herein provide methods of treating a disease or disorder provided herein (e.g., a disease associated with abnormal cell proliferation) comprising orally administering a cytidine analog provided herein (e.g., 5-azacytidine) for release substantially in the stomach, wherein the methods avoid adverse effects associated with administering a cytidine deaminase inhibitor (e.g., THU) by not co-administering the cytidine deaminase inhibitor with the cytidine analog. In particular embodiments, a cytidine deaminase inhibitor (e.g., THU) is co-administered with the cytidine analog in an amount of, e.g., less than about 500 mg/d, less than about 200 mg/d, less than about 150 mg/d, less than about 100 mg/d, less than about 50 mg/d, less than about 25 mg/d, less than about 10 mg/d, less than about 5 mg/d, less than about 1 mg/d, or less than about 0.1 mg/d.

In certain embodiments, methods provided herein comprise treating a disorder provided herein, including a hematologic disorder, by administering an oral dosage form comprising a cytidine analog to a subject in need thereof. In particular embodiments, oral dosage forms provided herein comprising 5-azacytidine are used to treat subjects having hematologic disorders. Hematologic disorders include, e.g., abnormal growth of blood cells which can lead to dysplastic changes in blood cells and hematologic malignancies such as various leukemias. Examples of hematologic disorders include, but are not limited to, acute myeloid leukemia (AML), acute promyelocytic leukemia (APML), acute lymphoblastic leukemia (ALL), chronic myelogenous leukemia (CML), chronic lymphocytic leukemia (CLL), myelodysplastic syndromes (MDS), and sickle cell anemia, among others. Other disorders that can be treated using the methods provided herein include, e.g., multiple myeloma (MM) and non-Hodgkin's lymphoma (NH L).

In certain embodiments, methods provided herein comprise treating AML by administering an oral dosage form comprising a cytidine analog to a subject in need thereof. AML is the most common type of acute leukemia that occurs in adults. Several inherited genetic disorders and immunodeficiency states are associated with an increased risk of AML. These include disorders with defects in DNA stability, leading to random chromosomal breakage, such as Bloom's syndrome, Fanconi's anemia, Li-Fraumeni kindreds, ataxia-telangiectasia, and X-linked agammaglobulinemia.

In certain embodiments, methods provided herein comprise treating APML by administering an oral dosage form comprising a cytidine analog to a subject in need thereof. APML represents a distinct subgroup of AML. This subtype is characterized by promyelocytic blasts containing the 15;17 chromosomal translocation. This translocation leads to the generation of the fusion transcript comprised of the retinoic acid receptor and a sequence PML.

In certain embodiments, methods provided herein comprise treating ALL by administering an oral dosage form comprising a cytidine analog to a subject in need thereof. ALL is a heterogenerous disease with distinct clinical features displayed by various subtypes. Reoccurring cytogenetic abnormalities have been demonstrated in ALL. The most common cytogenetic abnormality is the 9;22 translocation. The resultant Philadelphia chromosome represents poor prognosis of the subject.

In certain embodiments, methods provided herein comprise treating CML by administering an oral dosage form comprising a cytidine analog to a subject in need thereof. CML is a clonal myeloproliferative disorder of a pluripotent stem cell. CML is characterized by a specific chromosomal abnormality involving the translocation of chromosomes 9 and 22, creating the Philadelphia chromosome. Ionizing radiation is associated with the development of CML.

In certain embodiments, methods provided herein comprise treating MDS by administering an oral dosage form comprising a cytidine analog to a subject in need thereof. In certain embodiments, MDS includes one or more of the following myelodysplastic syndrome subtypes: refractory anemia, refractory anemia with ringed sideroblasts (if accompanied by neutropenia or thrombocytopenia or requiring transfusions), refractory anemia with excess blasts, refractory anemia with excess blasts in transformation, and chronic myelomonocytic leukemia. In certain embodiments, the MDS is higher-risk MDS. In certain embodiments, the methods provided herein comprise administering an oral dosage form comprising a cytidine analog to a subject in need thereof to increase the survival (e.g., prolong the life) of a subject with MDS.

In certain embodiments, methods provided herein comprise treating NHL by administering an oral dosage form comprising a cytidine analog to a subject in need thereof. Non-Hodgkin's Lymphomas (NHL) represent a heterogeneous group of malignancies of the lymphoid system. According to the WHO classification of hematological and lymphoid tumors, these diseases are classified as B-cell and T-cell neoplasms. B-cell lymphomas account for about 90% of all lymphomas, and the two most common histological disease entities are follicular lymphoma and diffuse large B-cell lymphoma. Approximately 55,000 to 60,000 new cases of NHL are diagnosed annually in the U.S. See, e.g., Ansell, S. M., et al., Mayo Clin. Proc., 2005, 80(8):1087-97.

In certain embodiments, methods provided herein comprise treating MM by administering an oral dosage form comprising a cytidine analog to a subject in need thereof. Multiple myeloma is one of the most commonly diagnosed hematologic malignancies. In 2007, in the U.S. alone, there were roughly 20,000 new MM cases and 10,000 deaths due to MM. The disease is characterized by, inter alia, an accumulation of malignant plasma cells in the bone marrow, which can lead to the overproduction of an immunoglobulin, e.g., a monoclonal immunoglobulin G or A. These immunoglobulins, also known as paraproteins, can be detected in the urine and blood of patients with MM. Consequences of MM include anemia, the development of destructive bony lesions, and renal insufficiency. See, e.g., Rao, K. V., American Journal of Health-System Pharmacy, 2007, 64(17): 1799-1807.

In certain embodiments, methods provided herein comprise treating CLL by administering an oral dosage form comprising a cytidine analog to a subject in need thereof. Chronic lymphocytic lymphoma (CLL) is a malignancy of mature B lymphocytes and is the most prevalent lymphoid malignancy in the U.S. The WHO classification of B lymphocytic neoplasms groups B cell malignancies according to the presumed normal counterpart of the malignant cells. CLL is diagnosed by immunophenotype analysis of lymphocytes from the blood, bone marrow, or lymph nodes. See, e.g., Zent, C. S., et al., Current Oncology Reports, 2007, 9:345-52.

Certain embodiments herein provide methods for delivering a cytidine analog to a subject comprising administering to the subject in need thereof an oral formulation comprising a cytidine analog. In particular embodiments, oral formulations comprise (1) a therapeutically effective amount of a cytidine analog; and (2) an optional drug release controlling component capable of releasing the cytidine analog substantially in the stomach after a subject ingests the oral formulation comprising the cytidine analog. Certain embodiments herein provide a method for enhancing the oral bioavailability of a cytidine analog in a subject. Certain embodiments herein provide a method of increasing the oral bioavailability of a cytidine analog comprising orally administering a pharmaceutical composition provided herein. In certain methods provided herein, a pharmaceutical composition provided herein is orally administered to a subject, contacts the biological fluids of the subject's body, and is absorbed in the upper gastrointestinal tract, such as, for example, substantially in the stomach.

Certain embodiments herein provide a method of achieving a particular exposure value provided herein by administering an oral formulation comprising a cytidine analog (e.g., 5-azacytidine) provided herein. Certain embodiments herein provide a method of achieving a particular oral bioavailability value provided herein by administering an oral formulation comprising a cytidine analog (e.g., 5-azacytidine) provided herein. Certain embodiments herein provide a method of achieving a particular AUC value provided herein by administering an oral formulation comprising a cytidine analog (e.g., 5-azacytidine) provided herein. Certain embodiments herein provide a method of achieving a particular Cmax value provided herein by administering an oral formulation comprising a cytidine analog (e.g., 5-azacytidine) provided herein. Certain embodiments herein provide a method of achieving a particular Tmax value provided herein by administering an oral formulation comprising a cytidine analog (e.g., 5-azacytidine) provided herein.

Certain embodiments herein provide methods of treating a condition involving undesirable or uncontrolled cell proliferation by administering an oral formulation comprising a cytidine analog (e.g., 5-azacytidine) as provided herein. Such conditions include, e.g., benign tumors, various types of cancers such as primary tumors and tumor metastasis, hematological disorders (e.g. leukemia, myelodysplastic syndrome and sickle cell anemia), restenosis (e.g. coronary, carotid, and cerebral lesions), abnormal stimulation of endothelial cells (arteriosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

In certain embodiments, cells in a benign tumor retain their differentiated features and do not divide in a completely uncontrolled manner. A benign tumor may be localized and/or nonmetastatic. Specific types of benign tumors that can be treated using the methods, compositions, and formulations provided herein include, e.g., hemangiomas, hepatocellular adenoma, cavernous hemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

In certain embodiments, cells in a malignant tumor become undifferentiated, do not respond to the body's growth control signals, and/or multiply in an uncontrolled manner. The malignant tumor may be invasive and capable of spreading to distant sites (metastasizing). Malignant tumors may be divided into two categories: primary and secondary. Primary tumors arise directly from the tissue in which they are found. A secondary tumor, or metastasis, is a tumor which is originated elsewhere in the body but has now spread to a distant organ. The common routes for metastasis are direct growth into adjacent structures, spread through the vascular or lymphatic systems, and tracking along tissue planes and body spaces (peritoneal fluid, cerebrospinal fluid, etc.).

Methylation can lead to the silencing of genes critical to cellular control (i.e., epigenetic gene silencing), and can be an early event in the development of malignant tumors including, e.g., colorectal cancer or lung cancer. See, e.g., M. V. Brock et al., N. Engl. J. Med., 2008, 358(11):1118-28; P. M. Das et al., Mol. Cancer, 2006, 5(28); G. Gifford et al., Clin. Cancer Res., 2004, 10:4420-26; J. G. Herman et al., N. Engl. J. Med., 2003, 349:2042-54; A. M. Jubb et al., J. Pathology, 2001, 195:111-34. Accordingly, in certain embodiments, methods herein provide using oral formulations provided herein to prevent or reverse epigenetic gene silencing, e.g., by reversing abnormal DNA methylation. In specific embodiments, oral formulations provided herein are used for early intervention to prevent the development of cancer in patients at risk of developing cancer, e.g., familial polyposis or lung cancer, wherein a cause of the cancer is epigenetic gene silencing. In particular embodiments, such early intervention would be impractical by means other than oral administration (e.g., IV or SC administration). In specific embodiments, oral formulations provided herein are used for early intervention to prevent the recurrence of cancer in patients at risk for early relapse, e.g., colorectal cancer or non-small-cell lung cancer. In certain embodiments, the early intervention is achieved via prolonged oral dosing schedules, using formulations and/or methods as described herein. Certain embodiments provide methods for administering oral formulations provided herein to reverse the effect of gene silencing, e.g., in patients at risk of gene silencing due to epigenetic changes. In particular embodiments, methods provided herein further comprise administering an HDAC inhibitor compound (e.g., to restore chromatin to a transcriptionally active configuration after reversing abnormal DNA methylation). In particular embodiments, the HDAC inhibitor compound is entinostat (SNDX-275; formerly MS-275), an oral HDAC inhibitor that acts synergistically with targeted therapies and is selective for cancer-relevant HDAC isoforms 1, 2, and 3. In particular embodiments, a synergistic effect is achieved by co-administering 5-azacytidine and an HDAC inhibitor (e.g., etinostat) for the treatment of solid tumors (e.g., NSCLC) or hematological malignancies (e.g., MDS, CMMoL, or AML).

In certain embodiments, specific types of cancers or malignant tumors, either primary or secondary, that can be treated using the methods, compositions, and formulations provided herein include, e.g., leukemia, breast cancer, skin cancer, bone cancer, prostate cancer, liver cancer, lung cancer (e.g., non-small-cell lung cancer and small-cell lung cancer), brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronmas, intestinal ganglioneuromas, hyperplastic corneal nerve tumor, marfanoid habitus tumor, Wilm's tumor, seminoma, ovarian tumor, leiomyoma tumor, cervical dysplasia and in situ carcinoma, neuroblastoma, retinoblastoma, medulloblastoma, soft tissue sarcoma, malignant carcinoid, topical skin lesion, mycosis fungoides, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas.

Particular embodiments herein provide using the methods, compositions, and formulations provided herein to treat abnormal cell proliferation due to, e.g., insults to body tissue during surgery for a variety of surgical procedures, including, e.g., joint surgery, bowel surgery, and cheloid scarring. Proliferative responses associated with organ transplantation that may be treated using the methods, compositions, and formulations provided herein include those proliferative responses contributing to potential organ rejections or associated complications. Specifically, these proliferative responses may occur during transplantation of the heart, lung (e.g., non-small-cell lung cancer and small-cell lung cancer), liver, kidney, and other body organs or organ systems.

In certain embodiments, the amount of the cytidine analog in the formulations provided herein, the methods of administration thereof, or the methods of treatment as set forth herein, is a specific dosage amount as provided herein. In certain embodiments, oral azacitidine dosages, methods of administration thereof, or methods of treatment of at least one condition, including but not limited to MDS and AML, may range, e.g., between about 50 mg/m2/day and about 2,000 mg/m2/day, between about 100 mg/m2/day and about 1,000 mg/m2/day, between about 100 mg/m2/day and about 500 mg/m2/day, or between about 120 mg/m2/day and about 250 mg/m2/day. In certain embodiments, particular dosages are, e.g., about 120 mg/m2/day, about 140 mg/m2/day, about 150 mg/m²/day, about 180 mg/m²/day, about 200 mg/m²/day, about 220 mg/m²/day, about 240 mg/m²/day, about 250 mg/m²/day, about 260 mg/m²/day, about 280 mg/m²/day, about 300 mg/m²/day, about 320 mg/m²/day, about 350 mg/m²/day, about 380 mg/m²/day, about 400 mg/m²/day, about 450 mg/m²/day, or about 500 mg/m² day.

In certain embodiments, appropriate biomarkers may be used to determine or predict the effect of the pharmaceutical compositions comprising cytidine analogs on the disease state and to provide guidance to the dosing schedule. For example, particular embodiments herein provide a method of determining whether a patient diagnosed with MDS has an increased probability of obtaining a greater benefit from treatment with a pharmaceutical composition comprising a cytidine analog by assessing the patient's nucleic acid methylation status. In particular embodiments, the cytidine analog is azacitidine. In particular embodiments, the nucleic acid is DNA or RNA. In particular embodiments, the greater benefit is an overall survival benefit. In particular embodiments, the methylation status is examined in one or more genes, e.g., genes associated with MDS or AML. Specific embodiments involve methods for determining whether baseline DNA methylation levels influence overall survival in patients with MDS (e.g., higher risk MDS) treated with azacitidine. Specific embodiments provide methods for determining whether gene promoter methylation levels influence overall survival in patients with MDS (e.g., higher risk MDS).

For example, specific embodiments herein provide methods for evaluating the influence of gene methylation on prolonged survival in patients with MDS (e.g., higher risk MDS). In particular embodiments, such evaluation is used to predict overall survival in patients with MDS (e.g., higher risk MDS), e.g., upon treatment with a pharmaceutical composition comprising a cytidine analog, as provided herein. In particular embodiments, such evaluation is used for therapeutic decision-making. In specific embodiments, such therapeutic decision-making includes planning or adjusting a patient's treatment, e.g., the dosing regimen, amount, and/or duration of administration of the cytidine analogue.

Certain embodiments provide methods of identifying individual patients diagnosed with MDS having an increased probability of obtaining an overall survival benefit from cytidine analog treatment, using analysis of methylation levels, e.g., in particular genes. In specific embodiments, lower levels of nucleic acid methylation are associated with an increased probability of obtaining improved overall survival following azacitidine treatment. In particular embodiments, the increased probability of obtaining improved overall survival following treatment is at least a 5% greater probability, at least a 10% greater probability, at least a 20% greater probability, at least a 30% greater probability, at least a 40% greater probability, at least a 50% greater probability, at least a 60% greater probability, at least a 70% greater probability, at least an 80% greater probability, at least a 90% greater probability, at least at least a 100% greater probability, at least a 125% greater probability, at least a 150% greater probability, at least a 175% greater probability, at least a 200% greater probability, at least a 250% greater probability, at least a 300% greater probability, at least a 400% greater probability, or at least a 500% greater probability of obtaining improved overall survival following treatment, e.g., using a pharmaceutical composition comprising a cytidine analog as provided herein. In particular embodiments, the greater probability of obtaining improved overall survival following treatment is a greater probability as compared to the average probability of a particular comparison population of patients diagnosed with MDS. In specific embodiments, the comparison population is a group of patients classified with a particular myelodysplastic subtype, as described herein. In one embodiment, the comparison population consists of patients having higher risk MDS. In particular embodiments, the comparison population consists of a particular IPSS cytogenetic subgroup.

In particular embodiments, nucleic acid (e.g., DNA or RNA) hypermethylation status may be determined by any method known in the art. In certain embodiments, DNA hypermethylation status may be determined using the bone marrow aspirates of patients diagnosed with MDS, e.g., by using quantitative real-time methylation specific PCR (“qMSP”). In certain embodiments, the methylation analysis may involve bisulfite conversion of genomic DNA. For example, in certain embodiments, bisulfite treatment of DNA is used to convert non-methylated CpG sites to UpG, leaving methylated CpG sites intact. See, e.g., Frommer, M., et al., Proc. Nat'l Acad. Sci. USA 1992, 89:1827-31. Commercially available kits may be used for such bisulfite treatment. In certain embodiments, to facilitate methylation PCR, primers are designed as known in the art, e.g., outer primers which amplify DNA regardless of methylation status, and nested primers which bind to methylated or non-methylated sequences within the region amplified by the first PCR. See, e.g., Li et al., Bioinformatics 2002, 18:1427-31. In certain embodiments, probes are designed, e.g., probes which bind to the bisulfite-treated DNA regardless of methylation status. In certain embodiments, CpG methylation is detected, e.g., following PCR amplification of bisulfite-treated DNA using outer primers. In certain embodiments, amplified product from the initial PCR reaction serves as a template for the nested PCR reaction using methylation-specific primers or non-methylation-specific primers. In certain embodiments, a standard curve is established to determine the percentage of methylated molecules in a particular sample. Methods for detecting nucleic acid methylation (e.g., RNA or DNA methylation) are known in art. See, e.g., Laird, P. W., Nature Rev. Cancer 2003, 3:253-66; Belinsky, S. A., Nature Rev. Cancer 2004, 4:1-11.

In certain embodiments, statistical analyses are performed to assess the influence of particular methylation levels with the potential benefit of treatment with a particular pharmaceutical composition comprising a cytidine analog. In certain embodiments, the influence of methylation on overall survival is assessed, e.g., using Cox proportional hazards models and Kaplan-Meier (KM) methodology.

In certain embodiments, any gene associated with MDS and/or AML may be examined for its methylation status in a patient. Particular genes include, but are not limited to, CKDN2B (p15), SOCS1, CDH1 (E-cadherin), TP73, and CTNNA1 (alpha-catenin). Particular genes associated with MDS and/or AML, which would be suitable for use in the methods disclosed here, are known in the art.

1. Methods Comprising Co-Administering One or More Additional Therapeutic Agents with the Oral Formulations Disclosed Herein

Certain embodiments herein provide methods of treating diseases or disorders disclosed herein (e.g., diseases or disorders involving abnormal cell proliferation), wherein the methods comprise co-administering an oral formulation disclosed herein (such as, for example, an oral formulation comprising 5-azacytidine) with one or more additional therapeutic agents (such as, for example, an anti-PD1/anti-PDL1 monoclonal antibody, e.g. pembrolizumab, MK-3475, pidilizumab, nivolumab (BMS-936558, MDX-1106, or ONO-4538), BMS-936559, atezolizumab (MPDL3280A), or durvalumab (MEDI4736) to yield a synergistic therapeutic effect. Particular co-administered therapeutic agents useful in the methods disclosed herein are disclosed throughout the specification. In particular embodiments, the additional therapeutic agent is co-administered in an amount that is a therapeutically effective amount. In particular embodiments, the additional therapeutic agent is co-administered in a separate dosage form from the cytidine analog dosage form with which it is co-administered. In particular embodiments, the additional therapeutic agent is co-administered in a dosage form (e.g., a single unit dosage form) together with the cytidine analog with which it is co-administered. In such cases, the cytidine analog (e.g., 5-azacytidine) and the additional therapeutic agent may be co-formulated together in the same dosage form using methods of co-formulating active pharmaceutical ingredients, including methods disclosed herein and methods known in the art.

### INCORPORATION BY REFERENCE

All disclosures (e.g., patents, publications, and web pages) referenced throughout this specification are incorporated by reference in their entireties. In addition, the following disclosures are also incorporated by reference herein in their entireties: (1) 2008 ASCO poster abstract by B. S. Skikne, M. R. Ward, A. Nasser, L. Aukerman, G. Garcia-Manero; (2) G. Garcia-Manero, M. L. Stoltz, M. R. Ward, H. Kantarjian, and S. Sharma, Leukemia, 2008, 22, 1680-84; and (3) WO 2009/139888.

### VI. Examples

### A. Example 1

A Phase 2 multicenter, randomized, double blind, placebo controlled study of immune checkpoint inhibition with pembrolizumab with or without epigenetic priming with oral 5-azacytidine in women with relapsed epithelial ovarian cancer is performed.

The current study will test the hypothesis that oral 5-azacytidine, an orally bioavailable formulation of AZA, can induce AIM expression in tumors of EOC patients and thereby enhance the response of these tumors to PD-1 inhibition with the monoclonal antibody pembrolizumab.

The objective of this study is to evaluate the activity and safety of pembrolizumab, alone and in combination with oral 5-azacytidine in patients with epithelial ovarian cancer.

Objectives

The primary objectives are to estimate progression-free survival (PFS) in both treatment arms and to estimate the PFS hazard ratio for the combination arm relative to the pembrolizumab monotherapy arm. The secondary objectives are to estimate the overall survival (OS), objective response rate (ORR), clinical benefit rate (CBR), and duration of clinical benefit in both treatment arms and evaluate safety. The exploratory objective is to assess the impact of AIM gene expression at baseline on response to pembrolizumab alone or in combination with oral 5-azacytidine.

Endpoints

The primary endpoint is the determination of PFS. The secondary endpoints are the determination of OS, ORR, CBR, duration of clinical benefit, and safety. The exploratory endpoints are the determination of AIM gene expression, locus specific methylation changes with oral 5-azacytidine dosing in circulating tumor DNA, quantification and characterization of tumor-infiltrating lymphocytes.

Study Design

The study will be a randomized, placebo-controlled, parallel group, multicenter double-blind phase 2 study. Subjects will be randomly assigned in a 1:1 ratio to one of two treatment arms:pembrolizumab as a 30 min i.v. infusion on Day 1 with oral 5-azacytidine (300 mg) or placebo administered orally on days 1-14 every 21 days. The study will be conducted under the guidance of a Steering Committee and an independent Data Monitoring Committee.

Subjects

Approximately 120 patients will be randomly assigned on a 1:1 basis between the two treatment arms over approximately 20 months. The main analysis will be performed when a total of approximately 80 PFS events have been observed. This number of events is expected to be observed after randomization of the last patient. At the time of the PFS analysis, all secondary endpoints will also be analyzed. A follow-up analysis for OS will be performed when 80 OS events have been observed after randomization of the last patient. Estimates for PFS, ORR, CBR, and duration of clinical benefit will be updated at the time of OS analysis.

Inclusion Criteria

The inclusion criteria for the study is as follows:
1. Subject is ≧18 years old at the time of signing ICF
2. Histologically documented papillary serous epithelial ovarian cancer
3. Documented relapse within 6 months after completing platinum doublet chemotherapy
4. ECOG performance status of 0-1
5. Measurable disease as per RECIST 1.1
6. Adequate organ functions:
   a. AST (SGOT), ALT (SGPT) ≦2.5× upper limit of normal range (ULN), or ≦5× ULN range if liver metastasis present
   b. Total bilirubin ≦1.5×ULN
   c. Creatinine ≦1.5×ULN
   d. Potassium within normal range, or correctable with supplements
7. Adequate bone marrow function:
   e. Absolute neutrophil count ≧1.5×10⁹ cells/L
   f. Platelets ≧100×10⁹ cells/L
   g. Hemoglobin ≧9 g/dL
      Females of child bearing potential must have a negative serum pregnancy test at screening and comply with pregnancy prevention requirements.

Exclusion Criteria

The exclusion criteria are as follows:
1. Serum albumin <3 g/dL
2. History of CNS metastases or carcinomatous meningitis
3. History of auto-immune disorders
4. History of pneumonitis or interstitial lung disease or any other medical condition that requires the use of steroids
5. History of clinically significant cardiac dysfunction or thromboembolic events
6. History of inflammatory bowel disease (e.g., Crohn's disease, ulcerative colitis), celiac disease, prior gastrectomy or upper bowel removal, or any other gastrointestinal disorder that would interfere with the absorption, distribution, metabolism or excretion of the study drug and/or predispose the subject to an increased risk of gastrointestinal toxicity
7. Major surgery ≦2 weeks prior to randomization or subject has not recovered from side effects of surgery.

Treatment Regimens

Monotherapy arm: pembrolizumab: administered as an i.v. infusion over 30 minutes at a dose of 10 mg/kg on day 1 every 21 days with matching oral 5-azacytidine placebo. Combination arm: pembrolizumab: administered as an i.v. infusion over 30 minutes at a dose of 10 mg/kg on day 1 every 21 day cycle with oral 5-azacytidine administered orally at a dose of 300 mg daily on days 1-14 of each 21 day. cycle.

Drug Supply Details

Pembrolizumab: Merck Corporation will supply pembrolizumab for IV administration. Pembrolizumab is provided as a white to off-white lyophilized powder in Type I glass vials intended for single use only. Pembrolizumab powder for Injection, 50 mg/vial, is reconstituted with sterile water for injection prior to use. The drug product is stored as a stable lyophilized powder under refrigerated conditions (2° C.-8° C.). 5-Azacytidine: Celgene Corporation will supply 5-azacytidine (or matching placebo) 100 mg tablets for oral administration. All tablets will be packaged in blister packs.

Treatment Duration

Subjects may be treated until radiologic disease progression (per RECIST 1.1), the subject begins a new anticancer treatment, withdrawal of consent, subject refusal, physician decision, toxicity that cannot be managed by dose delay or dose reduction, death, or the study ends for any reason.

Post Treatment Observation Period

Patients will be followed for survival every 12 weeks (telephone contact will be sufficient), or more frequent if requested. During this period, further anticancer therapy information (regimen, start and end date) will be collected. The median OS for patients with EOC who experience relapse within 6 months after a cisplatin duplet chemotherapy is approximately 12.0 months.

Assessments

Efficacy: Tumor assessment according to RECIST 1.1 will be performed every 6 weeks (±5 days) from randomization until documented disease progression, the subject begins a new anticancer treatment, withdrawal of consent, subject refusal, physician decision, toxicity that cannot be managed by dose delay or dose reduction, death, or the study ends for any reason. Safety: All subjects will be monitored for adverse events, starting from the time the subject signs the informed consent form until 28-days after the last dose of IP or the end of treatment (EOT) visit, whichever occurs later. A thorough evaluation of medical conditions will be conducted during screening for eligibility. Physical examination (source documented only), vital signs, laboratory assessments (e.g. serum chemistry, hematology), ECG, and ECOG performance status will be monitored regularly. Preventative measures will be taken to avoid pregnancy in trial subjects or their partners, and females of child-bearing potential will have regular pregnancy testing. Pharmacokinetics: Pharmacokinetic parameters will be studied. Biomarkers: A fresh biopsy must be collected during the screening period of 21 days prior to randomization from consented subjects. Blood and plasma samples will also be collected.

Statistical Methods

Since the primary objective of this study is to estimate instead of formally testing a hypothesis, the sample size of 120 was determined based on feasibility and not through a formal power calculation. The primary analysis will be performed when a total of approximately 80 PFS events have been documented. This is anticipated to occur after the randomization of the last subject. The confidence intervals for the hazard ratio between the combination arm and the pembrolizumab monotherapy arm can be calculated based on a few hypothetical hazard ratios. PFS will be summarized by median value using the Kaplan-Meier method. A Cox proportional hazard model will be used to estimate the hazard ratio (including a two sided 95% CI) between the combination arm and the pembrolizumab arms. Secondary endpoints of RR. CBR and duration of clinical benefit will be assessed at the time of the PFS analysis. OS will be reported when a total of 80 deaths have been reported. An updated analysis of other efficacy endpoints (PFS, RR, CBR, duration of clinical benefit) will also be presented at the time of OS analysis.

### B. Example 2

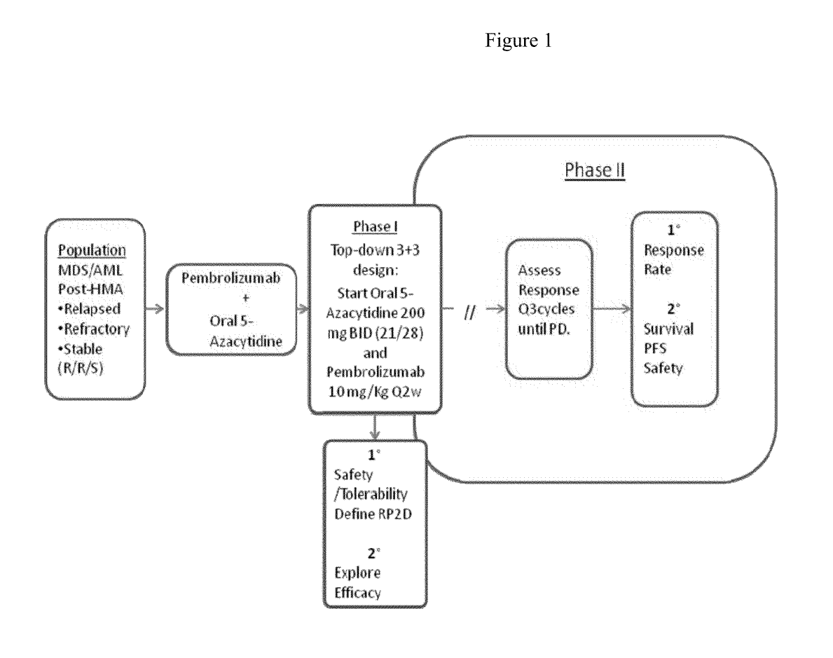

A Phase 1/2 international, multicenter, single-arm study of the safety and tolerability of oral 5-azacitidine in combination with pembrolizumab for the treatment of myelodysplastic syndromes (MDS) or acute myeloid leukemia (AML), not responding to treatment with injectable hypomethylating agents (HMAs), is performed. See FIG. 1 for study flow diagram.

Objectives

The primary objectives in Phase I are to evaluate the safety and tolerability of the regimen and to define the recommended Phase II dose (RP2D) for further evaluation. The primary objectives in Phase II are to evaluate efficacy measures associated with this regimen when used to treat patients with MDS or AML that did not respond to an injectable HMA. The secondary objectives in Phase I are to evaluate efficacy signals observed in these initial subjects. The secondary objectives in phase II are to evaluate safety and tolerability of the regimen when given in sequential treatment cycles. The exploratory objectives are to evaluate molecular and cellular markers in bone marrow and/or peripheral blood that may correlate with efficacy or resistance to the regimen.

Endpoints

The primary endpoints in Phase I are the determinations of dose-limiting toxicities, as well as number, type, and severity of other adverse events (AEs) reported. The primary endpoints in Phase II are the determinations of overall response rate. The secondary endpoints in Phase I are the determinations of number, type, and clinical relevance of objective responses. The secondary endpoints in Phase II are the determinations of survival, progression-free survival, duration of response, number, type, and severity of adverse events. The exploratory endpoints are biomarker assessments.

Study Design

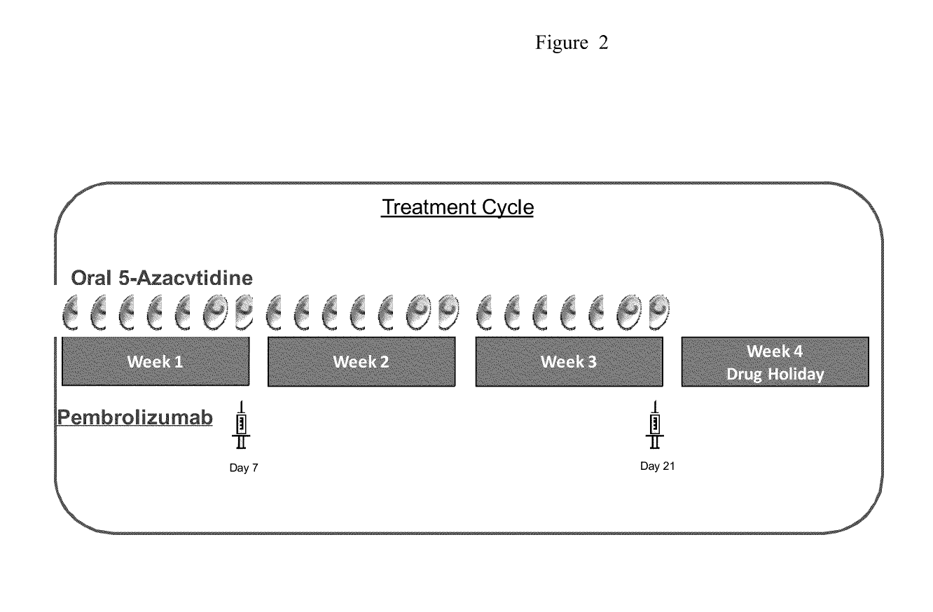

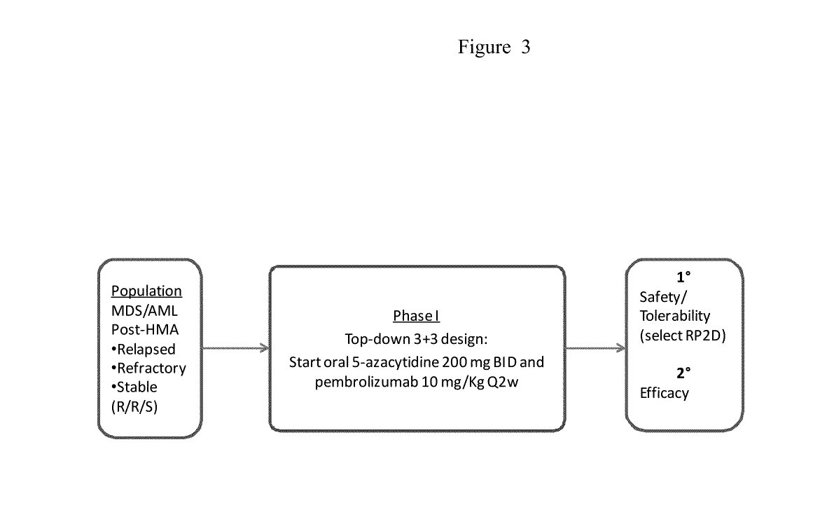

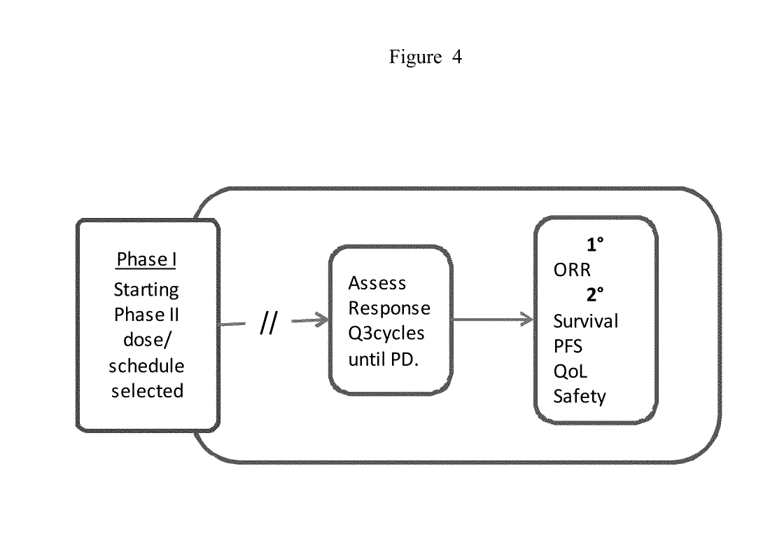

This is a Phase I/II uncontrolled study of the combination of oral 5-azacytidine with pembrolizumab for the treatment of patients with MDS or AML, who have not responded to treatment with an injectable hypomethylating agent (HMA). Phase I will seek to define the RP2D for the combination regimen and to explore biological and clinical responses to treatment. In Phase II, the sample size will be increased to seek proof of concept for the treatment of HMA-refractory MDS or AML and to build on the body of knowledge related to the safety and tolerability of the regimen. The study will initially enroll 3 subjects in Dosing Cohort 0 (oral 5-azacytidine, 200 mg BID 21/28 days and pembrolizumab 10 mg/Kg on cycle days 7 and 21 (or days 8 and 21)). See FIGS. 2, 3, and 4. If one of the initial 3 subjects experiences predefined dose-limiting toxicity (DLT) in the first treatment cycle, an additional 3 subjects will be enrolled in the cohort. If 2 or more first cycle DLTs are seen in any dosing cohort, the dose level will be declared intolerable and 3 subjects will be enrolled in next lower dosing cohort; e.g., Dosing Cohort −1. Otherwise the dosing cohort will be declared tolerable and will become the starting dose for further evaluation in Phase II. The 3-6 subjects receiving the RP2D in Phase I will be included in the Phase II portion of the study and will be evaluated for efficacy and safety as described for Phase II. A sufficient number of subjects will be included in Phase II. Subjects will start treatment at the RP2D from Phase I and may have their dose or schedule adjusted to address toxicity. Subjects will have a bone marrow evaluation following every 3 treatment cycles to look for signs of disease response or progression. Hematology parameters will be evaluated every cycle. If the subject experience a Complete Response (CR), dosing with pembrolizumab will be discontinued, while dosing with oral 5-azacytidine will continue. All treatment will be discontinued upon documented objective disease progression. Analysis of the primary endpoint, Overall Response Rate, will be conducted when the last active subject completes 6 cycles of treatment.

Inclusion Criteria The inclusion criteria for the study is as follows:
1. MDS (Int-1, Int-2, High) or AML
2. Prior injectable HMA (4 cycles vs. 6 months)
3. Progressive Disease or Stable Disease as the best response to iHMA
4. Documented objective PD/SD vs. “worsening cytopenias, increase in blasts, or progression in FAB subtype”
5. Last dose of iHMA within 12 weeks (≧3 weeks to C1D1)
6. ECOG 0, 1, 2

Exclusion Criteria

The exclusion criteria for the study is as follows:
1. Rapidly-progressing MDS (objective criteria vs. Inv. Judgment)
2. Prior oral decitabine
3. Prior or ongoing response to iHMA (if excluding relapse)
4. ESAs, TSAs, etc., hydroxyurea, other monoclonals, or live vaccine within 28 days
5. Other disease therapy within 14 days
6. Systemic corticosteroids within 7 days
7. GI disorders
8. Active CNS involvement
9. Autoimmune disorders or other requiring immunosuppression
10. Interstitial lung disease
11. Labs
   Bone marrow blasts >30(33)%
   WBC >20K/μL (30K)
   Serum creatinine >2.5×ULN (2×)
   Serum total bilirubin >1.5×ULN
   Serum aspartate aminotransferase (AST) or alanine aminotransferase (ALT) >2.5×ULN

Treatment Regimens Pembrolizumab: administered as an i.v. infusion over 30 minutes at a dose of 10 mg/kg on days 7 and 21 (or days 8 and 21) every 28 day cycle with oral 5-azacytidine administered orally at a dose of 200 mg BID on days 1-21 of each 28 day cycle. Further Phase I dosing cohorts are provided in Table 3 below.

TABLE 3
Phase I Dosing Cohorts

| Dose Level | Oral 5-Azacytidine | Pembrolizumab* |
|---|---|---|
| 0 | 200 mg BID (21/28) | 10 mg/Kg (D 7, D 21) |
| −1 | 150 mg BID (21/28) | 10 mg/Kg (D 7, D 21) |
| −2 | 100 mg BID (21/28) | 10 mg/Kg (D 7, D 21) |
| −3 | 200 mg QD (21/28) | 10 mg/Kg (D 7, D 21) |
| −4 | 200 mg QD (21/28) | 5 mg/Kg (D 7, D 21) |
| −5 | 200 mg QD (14/28) | 5 mg/Kg (D 7, D 21) |

*in the event of infusion reaction, infusion rate should be reduced

Treatment Duration

Subjects may be treated until CR, PD, withdrawal of consent, subject refusal, physician decision, toxicity that cannot be managed by dose delay or dose reduction, death, or the study ends for any reason. Re-treatment upon relapse from CR.

Post Treatment Observation Period

Q 6 weeks until PD or new therapy (clinic visit), then Q 12 weeks until death (telephone). Survival following disease progression after iHMA therapy is 4-6 months. This may be extended with either single-agent and we expect synergy from the combination.

Assessments

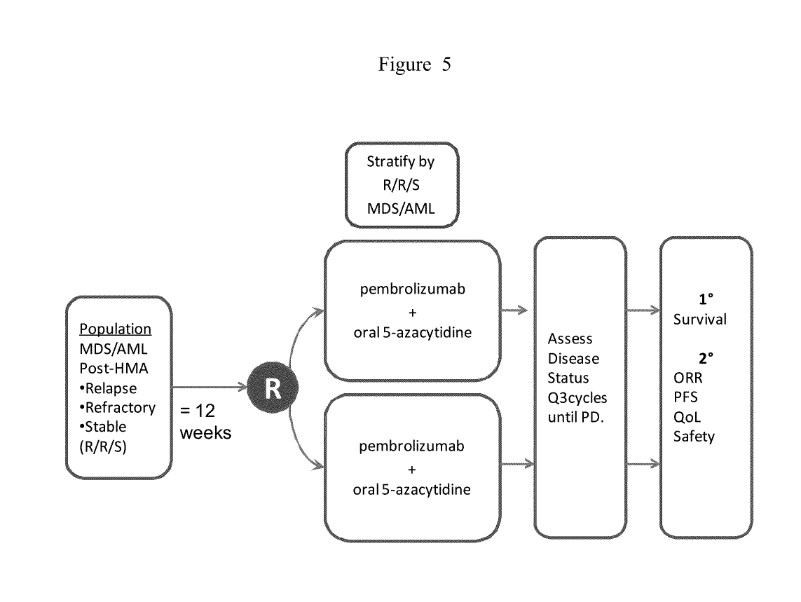

Efficacy: IWG 2006 response evaluation (bone marrow biopsy/aspirate) will be performed after completion of dosing in Cycle 2, Cycle 4, and Cycle 6, then after completion of dosing in every 3rd treatment cycle (9, 12, 15, etc.) to confirm suspected disease response or progression, and upon treatment discontinuation. Evaluation of hematology and hemoglobin will be performed after completion every cycle. Evaluation of quality of life (QoL) will be measured after every cycle. Evaluation of anti-pembrolizumab antibodies will be measured after every cycle. Safety: All subjects will be monitored for disease progression/transformation. Physical examination (source documented only), vital signs, laboratory assessments (e.g. serum chemistry, hematology), ECG, and ECOG performance status will be monitored regularly. Pharmacokinetics: Pharmacokinetic parameters will be studied. See FIG. 5.

### C. Example 3

A Phase 2 multicenter, randomized, double blind, placebo controlled study of oral 5-azacytidine in combination with pembrolizumab for the treatment of non-small cell lung cancer is performed.

Objectives

The primary objectives are to estimate progression-free survival (PFS) in both treatment arms and to estimate the PFS hazard ratio for the combination arm relative to the pembrolizumab monotherapy arm. The secondary objectives are to estimate the overall survival (OS), objective response rate (ORR), clinical benefit rate (CBR), and duration of clinical benefit in both treatment arms and evaluate safety.

Endpoints

The primary endpoint is the determination of PFS. The secondary endpoints are the determination of OS, ORR, CBR, duration of clinical benefit, and safety.

Study Design

The study will be a randomized, placebo-controlled, parallel group, multicenter double-blind phase 2 study. Subjects will be randomly assigned in a 1:1 ratio to one of two treatment arms:pembrolizumab as a 30 min i.v. infusion on Day 1 with oral 5-azacytidine (300 mg) or placebo administered orally on days 1-14 every 21 days. The study will be conducted under the guidance of a Steering Committee and an independent Data Monitoring Committee.

Subjects

Approximately 120 patients will be randomly assigned on a 1:1 basis between the two treatment arms over approximately 20 months. The main analysis will be performed when a total of approximately 80 PFS events have been observed. This number of events is expected to be observed after randomization of the last patient. At the time of the PFS analysis, all secondary endpoints will also be analyzed. A follow-up analysis for OS will be performed when 80 OS events have been observed after randomization of the last patient. Estimates for PFS, ORR, CBR, and duration of clinical benefit will be updated at the time of OS analysis.

### D. Example 4

Studies of the safety and tolerability of 5-azacitidine alone or in combination with durvalumab (MEDI4736) for the treatment of myelodysplastic syndromes (MDS) or acute myeloid leukemia (AML) are performed. Oral 5-azacytidine is used alone or in combination with durvalumab (MEDI4736) for patients with MDS who have not responded to treatment with hypomethylating agents (HMAs). Injectable 5-azacytidine is used alone or in combination with durvalumab (MEDI4736) for patients with untreated (first-line) higher risk MDS.

The first-line trial will also generate data from a randomized phase II cohort for the combination of azacitidine and durvalumab (MEDI4736) in elderly patients with untreated AML who are ineligible to receive allogeneic stem cell transplantation in preparation for a phase III study, or for accelerated approval in the USA if there is outstanding efficacy.

These two indications demonstrate high unmet medical need. The analysis of the available scientific data supports the hypothesis that the combination of either injectable 5-azacytidine or oral 5-azacytidine with durvalumab will be sufficiently synergistic to significantly improve the lives of patients with these diseases. Based on the limited knowledge currently available, the exploration of the combination of immune and epigenetic therapies is a high priority to improve the management of MDS.

Objective

The study will obtain initial combination data on safety, preliminary efficacy signals and possibly relevant biomark data.

Study Design

The first phase of the study includes three different clinical trials in three indications. Patients will be classified according to pregressive disease (PD) or stable disease (SD) at inclusion into the trial and evaluate response separately for both types.

In one trial, injectable 5-azacytidine will be administered in combination with durvalumab (MEDI4736) to patients who do not achieve overall response (OR) with durvalumab single agent. This would permit the generation of early safety and efficacy signals without competition with the other studies.

In one trial, oral 5-azacytidine will be administered, alone or in combination, with durvalumab in patients with higher risk MDS and HMA failure. This trial should generate the first efficacy signals and shall—via well defined parameters for go/no go decisions—demonstrate the efficacy of the combination therapy versus oral 5-azacytidine alone in a randomized phase II expansion phase. This trial will also aid with the planning of a subsequent phase III trial.

In one trial, a phase II trial with a safety run-in part in patients with previously untreated higher risk MDS to evaluate the combination of durvalumab and injectable 5-azacytidine. This trial should generate data in a population with better immune function and less resistant disease. In the same Celgene-sponsored trial the combination of injectable azacitidine and durvalumab versus injectable 5-azacytidine will be evaluated in randomized phase II design in a cohort of elderly patients with AML (WHO defined) who are not candidates for allogeneic stem cell transplantation.

5-Azacitidine will be administered to patients who do not respond (no CR, PR or HI after at least four cycles) or develop disease progression at any time to durvalumab in the current cohorts of lower risk and higher risk according to IPSS. Durvalumab will be administered at 10 mg/kg BW, every 2 weeks (Q2W); 5-azacytidine will be administered at 75 mg/m²/day, day 1 through day 7, every 4 weeks (Q4W). Alternatively, in one part oral 5-azacytidine will be administered 200 mg BID, 1-21 in 28 days (21/28 d, Q4W) to determine the safety of oral 5-azacytidine 200 mg BID in the patient group. In another part, oral 5-azacytidine will be administered 200 mg BID, 1-21 in 28 days (21/28 d, Q4W, de-escalation in the case of hematological or gastrointestinal toxicity), and durvalumab (1.5 g) will be administered every four weeks, with de-escalation if necessary.

There will be a dose de-escalation if DLT.

Treatment Duration

5-azacytidine will be administered until progression or intolerable toxicity. For durvalumab, subjects who achieve and maintain a complete response (CR), marrow complete remission (mCR), partial response (PR), SD, or hematological improvement (HI) through the end of the 12-month treatment period will enter follow-up. During the initial 12 months of follow-up, if a subject has PD, the subjects' study treatment may be re-administered for up to another 12 months with the same treatment guidelines followed during the initial 12-month period if the subject fulfills the criteria for retreatment in the setting of PD, has not received other anti-cancer treatments for their disease, and does not meet any of the investigational product discontinuation criteria for the study protocol. Only one round of retreatment will be allowed.

### E. Example 5

A phase 2, international, multicenter, randomized, open-label, parallel group study to evaluate the efficacy and safety of oral azacitidine alone and in combination with MEDI4736 (durvalumab) in subjects with myelodysplastic syndromes who fail to achieve an objective response to treatment with azacitidine for injection or decitabine.

Objective

The primary objective of this study is to investigate the efficacy of oral 5-azacytidine as monotherapy, and in combination with anti-PD-L1 monoclonal antibody, durvalumab, in subjects with MDS that did not respond to the most recent treatment with an injectable hypomethylating agent (HMA—azacitidine for injection or decitabine), or were unable to tolerate treatment with an injectable HMA. Further objectives are to assess the safety and tolerability of oral 5-azacytidine alone and in combination with durvalumab as treatment for MDS; to describe the clinical relevance of objective hematologic and/or biologic responses associated with treatment with oral 5-azacytidine alone and in combination with durvalumab; to determine the immunogenicity of durvalumab when given in combination with oral 5-azacytidine in subjects with MDS; and to evaluate the pharmacokinetics of durvalumab and oral 5-azacytidine in subjects with MDS.

Study Design

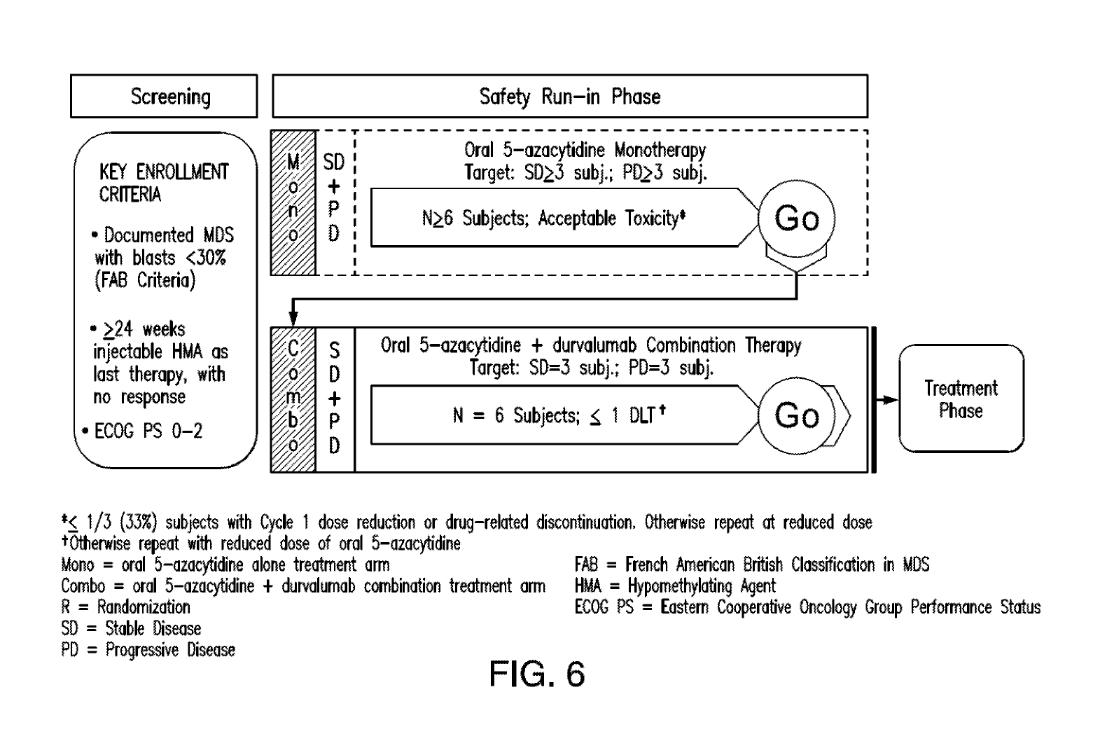

This is a Phase 2, international, multicenter, randomized, parallel group, open-label study consisting of 3 study phases: Screening, Randomized Treatment, and Follow-up. A Safety Run-in will explore the safety and tolerability of oral 5-azacytidine alone and in combination with durvalumab to confirm that there are no overlapping or synergistic toxicities limiting the ability of the two drugs to be delivered in combination (see FIG. 6). Once the acceptability of the combination regimen has been demonstrated, enrollment to the randomized treatment phase may begin.

Throughout the study, subjects will be categorized as having Progressive Disease (PD) or Stable Disease (SD) according to their response to iHMA therapy given as their most recent treatment for MDS. Numbers of subjects with PD and SD allocated to each treatment arm will be monitored to enable the planned analyses for each sub-population. Hence there are 4 cohorts being evaluated in this study:

Monotherapy, progressive disease
Monotherapy, stable disease
Combination therapy, progressive disease
Combination therapy, stable disease

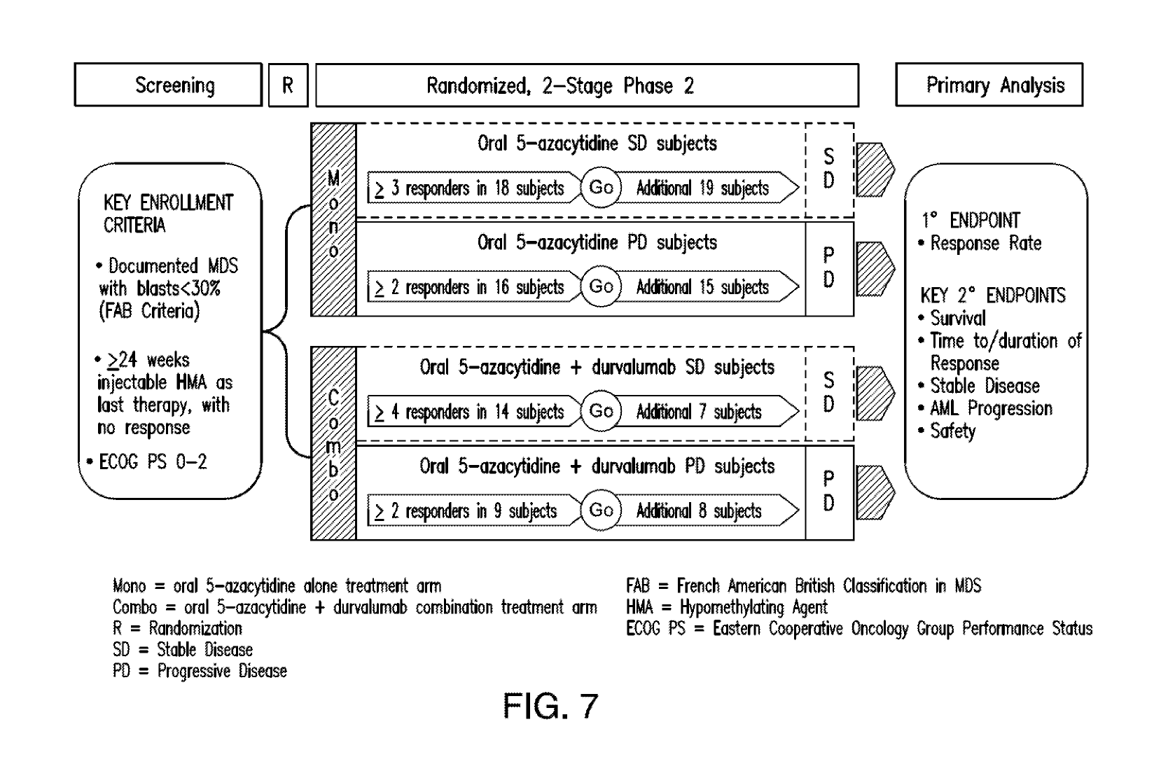

In the randomized treatment phase, eligible subjects will receive oral 5-azacytidine alone or in combination with durvalumab. The treatment phase will be conducted in 2 stages, with futility assessments for each of the 4 study cohorts determining whether that cohort proceeds to Stage 2 (see FIG. 7). The primary analysis will follow completion of Stage 2 and additional analyses will be conducted approximately 12 months after the last subject is enrolled.

This study will enroll approximately 69 to 130 subjects at up to approximately 75 investigational sites, worldwide. At least 12 subjects will be included in the safety run-in phase of the study: 6 receiving oral 5-azacytidine monotherapy, and 6 receiving oral 5-azacytidine+durvalumab combination therapy (see FIG. 6). The dose and schedule of oral 5-azacytidine in combination with a fixed dose of durvalumab will be determined.

Approximately 57 subjects will be enrolled in the first stage of the randomized treatment phase of the study. Approximately 16 subjects with progressive disease at study entry and 18 with stable disease at study entry will be randomized to receive oral 5-azacytidine monotherapy. Approximately 9 subjects with progressive disease at study entry and 14 with stable disease at study entry will be randomized to receive oral 5-azacytidine+durvalumab combination therapy. If sufficient objective responses are observed in one or more cohorts, the applicable cohort(s) will be expanded in Stage 2 to include approximately an additional 15 subjects with progressive disease and/or 19 subjects with stable disease in the monotherapy arm, and approximately 8 subjects with progressive disease and/or 7 subjects with stable disease in the combination therapy arm, for a maximum of approximately 130 subjects (see FIG. 7).

During the randomized treatment phase, PK sampling will be performed in approximately 10-12 subjects randomized to the combination therapy arm at selected sites with the required capabilities. This will enable assessment for potential drug-drug interactions, as well as effects of immunogenicity on PK parameters. All efforts will be made to include at least 5 subjects with PD and at least 5 subjects with SD at study entry.

Study Population

The study will enroll approximately 69 to 130 subjects with MDS who did not respond to treatment with an adequate course of therapy with an injectable hypomethylating agent (azacitidine for injection or decitabine) as their last treatment for MDS, or who were unable to tolerate treatment with an injectable HMA following at least 3 months of attempted treatment. At least 6 subjects will be enrolled in each of two treatment groups during the safety run-in phase of the study: Monotherapy (oral 5-azacytidine) or Combination Therapy (oral 5-azacytidine+durvalumab). Every attempt will be made to include at least 3 subjects with progressive disease (PD) and 3 subjects with stable disease (SD) in each treatment arm. Approximately 57 subjects will be enrolled in the first stage of the treatment phase. Enrollment will be monitored to ensure the proper numbers of subjects with PD and SD following iHMA therapy are included to assess futility in these sub-populations during Stage 1. If sufficient objective responses are observed in one or more cohorts (SD monotherapy, PD monotherapy, SD combination therapy, PD combination therapy), the applicable cohort(s) will be expanded in the second stage of study to include up to approximately 49 additional subjects. Again, enrollment will be monitored to ensure the proper numbers of subjects with PD and SD in each cohort to enable the planned analyses.

Length of Study

The total duration of this study is expected to be approximately 36 months. Subjects will undergo screening procedures over a period of up to 28 days following the signing of their informed consent document (ICD). Eligible subjects will continue to the safety run-in or the randomized treatment phase of the study where they will receive IP for up to six 28-day treatment cycles. Those who derive benefit from the treatment may continue IP until loss of that benefit. After treatment discontinuation, subjects will have a 28-day follow-up visit, then be contacted by telephone every 4 months in the follow-up phase of the study. The enrollment period for this study is expected to last approximately 24 months. The treatment and follow-up phases of study are expected to conclude approximately 12 months after the last subject is enrolled. Therefore, the total duration of the study is expected to be approximately 36 months. The End of Trial is defined as either the date of the last visit of the last subject to complete the study, or the date of receipt of the last data point from the last subject that is required for primary, secondary and/or exploratory analysis, as pre-specified in the protocol and/or the Statistical Analysis Plan (SAP), whichever is the later date.

Study Treatment (Investigational Product—IP)

(Oral Azacitidine) Eligible subjects who are assigned to the monotherapy treatment arm will receive oral 5-azacytidine at the dose and schedule identified during the safety run-in phase of the study. Dose and schedule may be adjusted in order to manage toxicity. Subjects who tolerate treatment with oral 5-azacytidine but show signs of worsening disease (clinical or hematological) or do not experience hematologic improvement or better (HI) following at least 2 well-tolerated treatment cycles may have their dose increased. Dose and schedule can again be reduced, but dose-escalation beyond 300 mg BID, 21/28 d may not be permitted. Subjects can continue treatment with oral 5-azacytidine as long as all protocol-specified re-treatment criteria continue to be met.

MEDI4736 (durvalumab) Eligible subjects who are assigned to the combination therapy treatment arm will receive oral 5-azacytidine as described above, and durvalumab 1500 mg on Day 1 of each 28-day treatment cycle by 1-hour intravenous (IV) infusion. For toxicity that is thought to be related to treatment with durvalumab, including immune-related AEs (irAEs) or infusion-type reactions, the infusion of durvalumab may be slowed or interrupted. Dose reduction for durvalumab may not be permitted. Subjects can continue treatment with durvalumab in combination with oral 5-azacytidine as long as all protocol-specified re-treatment criteria continue to be met. If treatment with oral 5-azacytidine is discontinued for any reason, dosing with durvalumab will be discontinued as well and the subject will enter the follow-up phase of the study.

Overview of Efficacy Assessments

The primary efficacy endpoint of this trial is the proportion of subjects achieving an objective response (HI, PR, CR, or marrow CR—modified from IWG 2006 criteria to treatment with oral 5-azacytidine alone and in combination with durvalumab. To assess this endpoint, bone marrow examination will be required prior to beginning IP and following every 2 cycles of treatment during the first 6 treatment cycles. Subjects who continue beyond Cycle 6 will undergo bone marrow examination following every 3 treatment cycles or when necessary to confirm suspected hematologic response or disease progression. Bone marrow samples (aspirate and/or biopsy), along with a peripheral blood smear and pertinent clinical information will be submitted for review by an independent pathologist to provide consistency in determinations of disease classification, response, and/or progression. Hematologic parameters including complete blood count (CBC) with white blood cell (WBC) differential and platelets will be assessed by the central laboratory. To understand the potential impact of any immunogenic response to durvalumab on the efficacy of the treatment, immunogenicity assessment will be performed.

Overview of Exploratory Assessments

Bone marrow and peripheral blood samples will be collected during screening, throughout the treatment phase of the study, and upon treatment discontinuation. These samples will be stored for potential future exploratory assessments of biological markers that may impact azacitidine efficacy (resistance to azacitidine for injection and/or oral 5-azacytidine resistance or sensitivity). Identifying biological markers predictive of treatment response or failure may enable a more targeted approach in the use of hypomethylating agent therapy for treatment of MDS patients based on their individual disease characteristics. Pharmacodynamic biomarkers of durvalumab therapy such as the levels of soluble PD-L1 and plasma cytokines/chemokines will be investigated in peripheral blood samples. Exploratory mechanistic and predictive biomarkers of response to oral 5-azacytidine/durvalumab combination therapy will be evaluated, including but not limited to PD-1/PD-L1 protein expression, gene expression signatures, circulating soluble proteins, genetic mutations and chromosomal aberrations, presence of tumor infiltrating lymphocytes, TCR clonality, and other checkpoint molecule expression (PD-L2, Tim-3, Lag-3, CTLA-4) as potential mechanisms of resistance.

All of the references cited herein are incorporated by reference in their entirety. While the methods provided herein have been described with respect to the particular embodiments, it will be apparent to those skilled in the art that various changes and modifications can be made without departing from the spirit and scope as recited by the appended claims.

The embodiments described above are intended to be merely exemplary and those skilled in the art will recognize or will be able to ascertain using no more than routine experimentation, numerous equivalents of specific compounds, materials and procedures. All such equivalents are considered to be within the scope and are encompassed by the appended claims.

What is claimed is:

1. A method for treating a subject having a disease or disorder, which comprises cyclically administering to the subject a therapeutically effective amount of 5-azacytidine, or a pharmaceutically acceptable salt, solvate or hydrate thereof, and a therapeutically effective amount of an anti-PD1 or anti-PDL1 monoclonal antibody, wherein the 5-azacytidine, or a pharmaceutically acceptable salt, solvate or hydrate thereof is administered orally.

2. The method of claim 1, where the disease or disorder is a solid tumor.

3. The method of claim 1, where the disease or disorder is a hematologic disorder.

4. The method of claim 1, wherein the disease or disorder is myelodysplastic syndromes, acute myeloid leukemia, ovarian cancer, or non-small cell lung cancer.

5. The method of claim 1, wherein the disease or disorder is relapsed or refractory.

6. The method of claim 1, wherein the subject having a disease or disorder did not respond to a prior treatment.

7. The method of claim 6, wherein the prior treatment comprises an injectable hypomethylating agent.

8. The method of claim 6, wherein the prior treatment comprises a platinum based regimen.

9. The method of claim 4, wherein the ovarian cancer is epithelial ovarian cancer.

10. The method of claim 9, wherein the epithelial ovarian cancer is relapsed epithelial ovarian cancer.

11. The method of claim 1, wherein the anti-PD1 monoclonal antibody is a humanized monoclonal IgG4 antibody.

12. The method of claim 11, wherein the humanized monoclonal IgG4 antibody is pembrolizumab, MK-3475, pidilizumab, Nivolumab (BMS-936558, MDX-1106, or ONO-4538).

13. The method of claim 1, wherein the anti-PDL1 monoclonal antibody is a humanized monoclonal IgG1 antibody.

14. The method of claim 13, wherein the IgG1 antibody is BMS-936559, atezolizumab (MPDL3280A), or durvalumab (MEDI4736).

15. The method of claim 1, wherein the 5-azacytidine, or a pharmaceutically acceptable salt, solvate or hydrate thereof is administered for 21 consecutive days followed by seven consecutive days of rest in a 28 day cycle.

16. The method of claim 1, wherein the 5-azacytidine, or a pharmaceutically acceptable salt, solvate or hydrate thereof is administered for 14 consecutive days followed by seven consecutive days of rest in a 21 day cycle.

17. The method of claim 1, wherein the 5-azacytidine, or a pharmaceutically acceptable salt, solvate or hydrate thereof is administered for 7 consecutive days followed by 21 consecutive days of rest in a 28 day cycle.

18. The method of claim 1, wherein the anti-PD1 or anti-PDL1 monoclonal antibody is administered on day 1 in a 28 day cycle, or on days 7 and 21 in a 28 day cycle.

19. The method of claim 1, wherein the anti-PD1 or anti-PDL1 monoclonal antibody is administered on days 8 and 21 in a 28 day cycle.

20. The method of claim 1, wherein the anti-PD1 or anti-PDL1 monoclonal antibody is administered on day 1 in a 21 day cycle.

21. The method of claim 1, wherein the 5-azacytidine, or a pharmaceutically acceptable salt, solvate or hydrate thereof is administered for 21 consecutive days followed by seven consecutive days of rest in a 28 day cycle, and wherein the anti-PD1 or anti-PDL1 monoclonal antibody is administered on days 7 and 21 of the 28 day cycle, or wherein the anti-PD1 or anti-PDL1 monoclonal antibody is administered on day 1 of the 28 day cycle.

22. The method of claim 1, wherein the 5-azacytidine, or a pharmaceutically acceptable salt, solvate or hydrate thereof is administered for 21 consecutive days followed by seven consecutive days of rest in a 28 day cycle, and wherein the anti-PD1 or anti-PDL1 monoclonal antibody is administered on days 8 and 21 of the 28 day cycle.

23. The method of claim 1, wherein the 5-azacytidine, or a pharmaceutically acceptable salt, solvate or hydrate thereof is administered for 14 consecutive days followed by seven consecutive days of rest in a 21 day cycle, and wherein the anti-PD1 or anti-PDL1 monoclonal antibody is administered on day 1 of the 21 day cycle.

24. The method of claim 1, wherein the 5-azacytidine, or a pharmaceutically acceptable salt, solvate or hydrate thereof and the anti-PD1 or anti-PDL1 monoclonal antibody are administered until disease progression or unacceptable toxicity.

25. The method of claim 1, wherein the 5-azacytidine or a pharmaceutically acceptable salt, solvate or hydrate thereof is administered in an amount of about 50 mg, about 100 mg, about 200 mg, about 300 mg, about 400 mg, about 500 mg, or about 600 mg per day.

26. The method of claim 1, wherein the 5-azacytidine or a pharmaceutically acceptable salt, solvate or hydrate thereof is administered twice per day.

27. The method of claim 26, wherein the 5-azacytidine or a pharmaceutically acceptable salt, solvate or hydrate thereof is administered in an amount of about 100 mg, 150 mg, or 200 mg twice per day.

28. The method of claim 1, wherein the 5-azacytidine or a pharmaceutically acceptable salt, solvate or hydrate thereof is in a form of a capsule, tablet or caplet.

29. The method of claim 1, wherein the anti-PD1 or anti-PDL1 monoclonal antibody is administered parenterally.

30. The method of claim 1, wherein the anti-PD1 or anti-PDL1 monoclonal antibody is administered in an amount of about 1,500 mg per day, or about 1 mg/Kg, about 2 mg/Kg, about 3 mg/Kg, about 4 mg/Kg, about 5 mg/Kg, about 6 mg/Kg, about 7 mg/Kg, about 8 mg/Kg, about 9 mg/Kg, about 10 mg/Kg, about 11 mg/Kg, about 12 mg/Kg, about 13 mg/Kg, about 14 mg/Kg, about 15 mg/Kg, about 16 mg/Kg, about 17 mg/Kg, about 18 mg/Kg, about 19 mg/Kg, or about 20 mg/Kg per day.

31. The method of claim 30, wherein the anti-PD1 or anti-PDL1 monoclonal antibody is administered in an amount of about 1,500 mg per day on day 1 in a 28 day cycle, or about 10 mg/Kg on days 7 and 21 in a 28 day cycle.

32. The method of claim 30, wherein the anti-PD1 or anti-PDL1 monoclonal antibody is administered in an amount of about 10 mg/Kg on days 8 and 21 in a 28 day cycle.

33. The method of claim 30, wherein the anti-PD1 or anti-PDL1 monoclonal antibody is administered in an amount of about 10 mg/Kg on day 1 in a 21 day cycle.

34. The method of claim 21, wherein the disease or disorder is myelodysplastic syndromes or acute myeloid leukemia.

35. The method of claim 22, wherein the disease or disorder is myelodysplastic syndromes or acute myeloid leukemia.

36. The method of claim 23, wherein the disease or disorder is ovarian cancer or non-small cell lung cancer.

37. The method of claim 36, wherein the ovarian cancer is epithelial ovarian cancer.

38. The method of claim 1, which further comprises administering a therapeutically effective amount of an additional active agent.

39. The method of claim 1, wherein the subject is a human.
